SEQUENCE LISTING (38 sequences)

SEQ ID NO: 1 - protein, 5 aa, Mus sp.
DYSMH

SEQ ID NO: 2 - protein, 17 aa, Mus sp.
WINTETGEPTYADDFKG

SEQ ID NO: 3 - protein, 13 aa, Mus sp.
PYYDYVSYYAMDY

SEQ ID NO: 4 - protein, 122 aa, Mus sp.
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 5 - protein, 122 aa, synthetic construct
  REGION 1..122: note = Description of Artificial Sequence: Synthetic polypeptide
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 6 - DNA, 458 bp, synthetic construct
  misc_feature 1..458: note = Description of Artificial Sequence: Synthetic polynucleotide
  source 1..458: mol_type = other DNA
actagtaccaccatggcttgggtgtggaccttgctattcctgatggcagctgcccaaagtatccaagcacaggttcagttggtgcagtctggatctgagctgaagaagcctggagcctcagtcaaggtttcctgcaaggcttctggttataccttcacagactattcaatgcactgggtgcgacaggctccaggacaaggtttaaagtggatgggctggataaacactgagactggtgagccaacatatgcagatgacttcaagggacggtttgtcttctctttggacacctctgtcagcactgcctatttgcagatcagcagcctcaaagctgaggacacggctgtgtattactgtgctaatccctactatgattacgtctcttactatgctatggactactggggtcagggaaccacggtcaccgtctcctcaggtaagaatggcctctcaagctt

SEQ ID NO: 7 - protein, 11 aa, Mus sp.
KASQDVSTAVA

SEQ ID NO: 8 - protein, 7 aa, Mus sp.
SASYLYT

SEQ ID NO: 9 - protein, 9 aa, Mus sp.
QQHYSTPRT

SEQ ID NO: 10 - protein, 107 aa, Mus sp.
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 11 - protein, 107 aa, synthetic construct
  REGION 1..107: note = Description of Artificial Sequence: Synthetic polypeptide
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 12 - DNA, 416 bp, synthetic construct
  misc_feature 1..416: note = Description of Artificial Sequence: Synthetic polynucleotide
  source 1..416: mol_type = other DNA
gctagcaccaccatggagtcacagattcaggtctttgtattcgtgtttctctggttgtctggtgttgacggagacattcagatgacccagtctccatcctccctgtccgcatcagtgggagacagggtcaccatcacctgcaaggccagtcaggatgtgagtactgctgtagcctggtatcaacagaaaccaggaaaagcccctaaactactgatttactcggcatcctacctctacactggagtcccttcacgcttcagtggcagtggatctgggacggatttcactttcaccatcagcagtctgcagcctgaagacattgcaacatattactgtcagcaacattatagtactcctcggacgttcggtcagggcaccaagctggaaatcaaacgtaagtagaatccaaagaattc

SEQ ID NO: 13 - protein, 5 aa, Mus sp.
SHDMS

SEQ ID NO: 14 - protein, 17 aa, Mus sp.
AINSDGGSTYYPDTMER

SEQ ID NO: 15 - protein, 11 aa, Mus sp.
HYDDYYAWFAY

SEQ ID NO: 16 - protein, 120 aa, Mus sp.
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 17 - protein, 120 aa, synthetic construct
  REGION 1..120: note = Description of Artificial Sequence: Synthetic polypeptide
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 18 - DNA, 451 bp, synthetic construct
  misc_feature 1..451: note = Description of Artificial Sequence: Synthetic polynucleotide
  source 1..451: mol_type = other DNA
actagtaccaccatggacttcgggctcagcttggttttccttgtccttattttaaaaagtgtacagtgtgaggtgcagctggtggagtctgggggaggcttagtgcagcctggagggtccctgagactctcctgtgcagcctctgaatacgagttcccttcccatgacatgtcttgggtccgccaggctccggggaaggggctggagttggtcgcagccattaatagtgatggtggtagcacctactatccagacaccatggagagacgattcaccatctccagagacaatgccaagaactcactgtacctgcaaatgaacagtctgagggccgaggacacagccgtgtattactgtgcaagacactatgatgattactacgcctggtttgcttactggggccaagggactatggtcactgtctcttcaggtgagtcctaacttcaagctt

SEQ ID NO: 19 - protein, 15 aa, Mus sp.
RASKSVSTSGYSYMH

SEQ ID NO: 20 - protein, 7 aa, Mus sp.
LASNLES

SEQ ID NO: 21 - protein, 9 aa, Mus sp.
QHSRELPLT

SEQ ID NO: 22 - protein, 111 aa, Mus sp.
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 23 - protein, 111 aa, synthetic construct
  REGION 1..111: note = Description of Artificial Sequence: Synthetic polypeptide
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 24 - DNA, 428 bp, synthetic construct
  misc_feature 1..428: note = Description of Artificial Sequence: Synthetic polynucleotide
  source 1..428: mol_type = other DNA
gctagcaccaccatggagacagacacactcctgttatgggtactgctgctctgggttccaggttccactggtgaaattgtgctgacacagtctcctgctaccttatctttgtctccaggggaaagggccaccctctcatgcagggccagcaaaagtgtcagtacatctggctatagttatatgcactggtaccaacagaaaccaggacaggctcccagactcctcatctatcttgcatccaacctagaatctggggtccctgccaggttcagtggcagtgggtctgggacagacttcaccctcaccatcagcagcctagagcctgaggattttgcagtttattactgtcagcacagtagggagcttccgctcacgttcggcggagggaccaaggtcgagatcaaacgtaagtacacttttctgaattc

SEQ ID NO: 25 - protein, 5 aa, Mus sp.
DAWMD

SEQ ID NO: 26 - protein, 19 aa, Mus sp.
EIRSKANNHATYYAESVNG

SEQ ID NO: 27 - protein, 8 aa, Mus sp.
GEVFYFDY

SEQ ID NO: 28 - DNA, 414 bp, Mus sp.
  source 1..414: mol_type = genomic DNA
atgtacttgggactgaactatgtattcatagtttttctcttaaatggtgtccagagtgaagtgaagcttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctca

SEQ ID NO: 29 - protein, 138 aa, Mus sp.
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 30 - DNA, 448 bp, synthetic construct
  misc_feature 1..448: note = Description of Artificial Sequence: Synthetic polynucleotide
  source 1..448: mol_type = other DNA
actagtaccaccatgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcaggtgagtccttaaaacaagctt

SEQ ID NO: 31 - protein, 138 aa, synthetic construct; identical to SEQ ID NO: 29
  REGION 1..138: note = Description of Artificial Sequence: Synthetic polypeptide

SEQ ID NO: 32 - protein, 11 aa, Mus sp.
KSSQDINKYIA

SEQ ID NO: 33 - protein, 7 aa, Mus sp.
YTSTLQP

SEQ ID NO: 34 - protein, 8 aa, Mus sp.
LQYDNLLT

SEQ ID NO: 35 - DNA, 378 bp, Mus sp.
  source 1..378: mol_type = genomic DNA
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaa

SEQ ID NO: 36 - protein, 126 aa, Mus sp.
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 37 - DNA, 413 bp, synthetic construct
  misc_feature 1..413: note = Description of Artificial Sequence: Synthetic polynucleotide
  source 1..413: mol_type = other DNA
gctagcaccaccatgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgtaagtacacttttctgaattc

SEQ ID NO: 38 - protein, 126 aa, synthetic construct; identical to SEQ ID NO: 36
  REGION 1..126: note = Description of Artificial Sequence: Synthetic polypeptide

(19) United States
(12) Patent Application Publication
(10) Pub. No.: US 2023/0094076 A1
(43) Pub. Date: Mar. 30, 2023

(54) COMBINATION THERAPY

(71) Applicant: GlaxoSmithKline Intellectual Property Development Limited, Middlesex (GB)

(72) Inventors: Olena I. BARBASH, Collegeville, PA (US); Andy FEDORIW, Collegeville, PA (US); Susan KORENCHUK, Collegeville, PA (US); Helai MOHAMMAD, Collegeville, PA (US); Christian SHERK, Collegeville, PA (US)

(21) Appl. No.: 17/932,986

(22) Filed: Sep. 16, 2022

Related U.S. Application Data
(63) Continuation of application No. 16/465,349, filed on May 30, 2019, now abandoned.
(60) Provisional application No. 62/428,757, filed on Dec. 1, 2016.
(60) Provisional application No. 62/433,359, filed on Dec. 13, 2016.

(51) Int. Cl. A61K 31/415; A61P 35/00; A61K 9/00; A61K 39/395

(52) U.S. Cl. CPC A61K 31/415; A61P 35/00; A61K 9/0053; A61K 39/3955; A61K 2039/505

(57) ABSTRACT

In one embodiment, the present invention provides a combination of a Type I protein arginine methyltransferase (Type I PRMT) inhibitor and an immuno-modulatory agent selected from: an anti-PD-1 antibody or antigen binding fragment thereof, an anti-PDL1 antibody or antigen binding fragment thereof, and anti-OX40 antibody or antigen binding fragment thereof. In another embodiment, the present invention provides a pharmaceutical composition comprising a therapeutically effective amount of a Type I protein arginine methyltransferase (Type I PRMT) inhibitor and a second pharmaceutical composition comprising a therapeutically effective amount of an immuno-modulatory agent selected from: anti-PD-1 antibody or antigen binding fragment thereof, an anti-PDL1 antibody or antigen binding fragment thereof, and an anti-OX40 antibody or antigen binding fragment thereof. In another embodiment, methods for treating cancer in a human in need thereof are provided, the methods comprising administering to the human the combinations or pharmaceutical compositions provided herein.

### CROSS-REFERENCE TO RELATED APPLICATIONS

This application is a continuation of U.S. application Ser. No. 16/465,349, filed May 30, 2019, which is a 371 U.S. National Stage of International Application No. PCT/IB2017/057548, filed Nov. 30, 2017, which claims priority of U.S. Provisional Application Nos. 62/428,757, filed Dec. 1, 2016, and 62/433,359, filed Dec. 13, 2016, the entireties of which are hereby incorporated by reference.

### SEQUENCE LISTING

This application contains a Sequence Listing that has been submitted electronically as an XML file named “26368-0125002.xml.” The XML file, created on Sep. 16, 2022, is 43,383 bytes in size. The material in the XML file is hereby incorporated by reference in its entirety.

### FIELD OF THE INVENTION

The present invention relates to a method of treating cancer in a mammal and to combinations useful in such treatment. In particular, the present invention relates to combinations of Type I protein arginine methyltransferase (Type I PRMT) inhibitors and immuno-modulatory agents, such as anti-PD-1 and anti-OX40 antibodies.

### BACKGROUND OF THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

Arginine methylation is an important post-translational modification on proteins involved in a diverse range of cellular processes such as gene regulation, RNA processing, DNA damage response, and signal transduction. Proteins containing methylated arginines are present in both nuclear and cytosolic fractions suggesting that the enzymes that catalyze the transfer of methyl groups on to arginines are also present throughout these subcellular compartments (reviewed in Yang, Y. & Bedford, M. T. Protein arginine methyltransferases and cancer. Nat Rev Cancer 13, 37-50, doi: 10.1038/nrc3409 (2013); Lee, Y. H. & Stallcup. M. R. Minireview: protein arginine methylation of nonhistone proteins in transcriptional regulation. Mol Enclocrinol 23, 425-433, doi:10.1210/me.2008-0380 (2019)). In mammalian cells, methylated arginine exists in three major forms: ω-N^{G}-monomethyl-arginine (MMA), ω-N^{G},N^{G}-asymmetric dimethyl arginine (ADMA), or ω-N^{G},N′^{G}-symmetric dimethyl arginine (SDMA). Each methylation state can affect protein-protein interactions in different ways and therefore has the potential to confer distinct functional consequences for the biological activity of the substrate (Yang, Y. & Bedford, M. T. Protein arginine methyltransferases and cancer. Nat Rev Cancer 13, 37-50, doi:10.1038/nrc3409 (2013)).

Arginine methylation occurs largely in the context of glycine-, arginine-rich (GAR) motifs through the activity of a family of Protein Arginine Methyltransferases (PRMTs) that transfer the methyl group from S-adenosyl-L-methionine (SAM) to the substrate arginine side chain producing S-adenosyl-homocysteine (SAH) and methylated arginine. This family of proteins is comprised of 10 members of which 9 have been shown to have enzymatic activity (Bedford, M. T. & Clarke, S. G. Protein arginine methylation in mammals: who, what, and why. Mol Cell 33, 1-13, doi: 10.1016/j.molcel.2008.12.013 (2009)). The PRMT family is categorized into four sub-types (Type I-IV) depending on the product of the enzymatic reaction. Type IV enzymes methylate the internal guanidino nitrogen and have only been described in yeast (Fisk, J. C. & Read, L. K. Protein arginine methylation in parasitic protozoa Eukaryot Cell 10, 1013-1022, doi:10.1128/EC.05103-11 (2011)); types I-III enzymes generate monomethyl-arginine (MMA, Rme1) through a single methylation event. The MMA intermediate is considered a relatively low abundance intermediate, however, select substrates of the primarily Type III activity of PRMT7 can remain monomethylated, while Types I and II enzymes catalyze progression from MMA to either asymmetric dimethyl-arginine (ADMA, Rme2a) or symmetric dimethyl arginine (SDMA, Rme2s) respectively. Type II PRMTs include PRMT5, and PRMT9, however, PRMT5 is the primary enzyme responsible for formation of symmetric dimethylation. Type I enzymes include PRMT1, PRMT3, PRMT4, PRMT6 and PRMT8. PRMT1, PRMT3, PRMT4, and PRMT6 are ubiquitously expressed while PRMT8 is largely restricted to the brain (reviewed in Bedford, M. T. & Clarke, S. G. Protein arginine methylation in mammals: who, what, and why. Mol Cell 33, 1-13, doi:10.1016/j.molcel.2008.12.013 (2009)).

Mis-regulation and overexpression of PRMT1 has been associated with a number of solid and hematopoietic cancers (Yang, Y. & Bedford, M. T. Protein arginine methyltransferases and cancer. Nat Rev Cancer 13, 37-50, doi:10.1038/nrc3409 (2013); Yoshimatsu, M. et al. Dysregulation of PRMT1 and PRMT6, Type I arginine methyltransferases, is involved in various types of human cancers. Int J Cancer 128, 562-573, doi: 10.1002/ijc.25366 (2011)). The link between PRMT1 and cancer biology has largely been through regulation of methylation of arginine residues found on relevant substrates. In several tumor types, PRMT1 can drive expression of aberrant oncogenic programs through methylation of histone H4 (Takai, H. et al. 5-Hydroxymethylcytosine plays a critical role in glioblastomagenesis by recruiting the CHTOP-methylosome complex. Cell Rep 9, 48-60, doi:10.1016/j.celrep.2014.08.071 (2014); Shia, W. J. et al. PRMT1 interacts with AML1-ETO to promote its transcriptional activation and progenitor cell proliferative potential. Blood 119, 4953-4962, doi:10.1182/blood-2011-04-347476 (2012); Zhao, X. et al. Methylation of RUNX1 by PRMT1 abrogates SIN3A binding and potentiates its transcriptional activity. Genes Dev 22, 640-653, doi:10.1101/gad.1632608 (2008), as well as through its activity on non-histone substrates (Wei, H., Mundade, R., Lange, K. C. & Lu, T. Protein arginine methylation of non-histone proteins and its role in diseases. Cell Cycle 13, 32-41, doi:10.4161/cc.27353 (2014)). In many of these experimental systems, disruption of the PRMT1-dependent ADMA modification of its substrates decreases the proliferative capacity of cancer cells (Yang, Y. & Bedford, M. T. Protein arginine methyltransferases and cancer. Nat Rev Cancer 13, 37-50, doi:10.1038/nrc3409 (2013)). Accordingly, it has been recognized that an inhibitor of PRMT1 should be of value both as an anti-proliferative agent for use in the treatment of hyperproliferative disorders.

Immunotherapies are another approach to treat hyperproliferative disorders. Enhancing anti-tumor T cell function and inducing T cell proliferation is a powerful and new approach for cancer treatment. Three immune-oncology antibodies (e.g., immuno-modulators) are presently marketed. Anti-CTLA-4 (YERVOY®/ipilimumab) is thought to augment immune responses at the point of T cell priming and anti-PD-1 antibodies (OPDIVO®/nivolumab and KEYTRUDA®/pembrolizumab) are thought to act in the local tumor microenvironment, by relieving an inhibitory checkpoint in tumor specific T cells that have already been primed and activated.

Though there have been many recent advances in the treatment of cancer, there remains a need for more effective and/or enhanced treatment of an individual suffering the effects of cancer.

### BRIEF DESCRIPTION OF THE DRAWINGS

FIG. 1: Types of methylation on arginine residues. From Yang, Y. & Bedford, M. T. Protein arginine methyltransferases and cancer. Nat Rev Cancer 13, 37-50, doi: 10.1038/nrc3409 (2013).

FIG. 2: Functional classes of cancer relevant PRMT1 substrates. Known substrates of PRMT1 and their association to cancer related biology (Yang, Y. & Bedford, M. T. Protein arginine methyltransferases and cancer. Nat Rev Cancer 13, 37-50, doi:10.1038/nrc3409 (2013): Shia, W. J. et al. PRMT1 interacts with AML1-ETO to promote its transcriptional activation and progenitor cell proliferative potential. Blood 119, 4953-4962, doi:10.1182/blood-2011-04-347476 (2012); Wei, H., Mundade, R., Lange, K. C. & Lu, T. Protein arginine methylation of non-histone proteins and its role in diseases. Cell Cycle 13, 32-41, doi:10.4161/cc.27353 (2014); Boisvert, F. M., Rhie, A., Richard, S. & Doherty, A. J. The GAR motif of 53BP1 is arginine methylated by PRMT1 and is necessary for 53BP1 DNA binding activity. Cell Cycle 4, 1834-1841, doi:10.4161/cc.4.12.2250 (2005); Boisvert, F. M., Dery, U., Masson, J. Y. & Richard, S. Arginine methylation of MRE11 by PRMT1 is required for DNA damage checkpoint control. Genes Dev 19, 671-676, doi:10.1101/gad.1279805 (2005); Zhang, L. et al. Cross-talk between PRMT1-mediated methylation and ubiquitylation on RBM15 controls RNA splicing. Elife 4, doi:10.7554/eLife.07938 (2015); Snijders, A. P. et al. Arginine methylation and citrullination of splicing factor proline- and glutamine-rich (SFPQ/PSF) regulates its association with mRNA. RNA 21, 347-359, doi:10.1261/rna.045138.114 (2015); Liao, H. W. et al. PRMT1-mediated methylation of the EGF receptor regulates signaling and cetuximab response. J Clin Invest 125, 4529-4543, doi:10.1172/JCI82826 (2015); Ng, R. K. et al. Epigenetic dysregulation of leukaemic HOX code in MLL-rearranged leukaemia mouse model. J Pathol 232, 65-74, doi:10.1002/path.4279 (2014); Bressan. G. C. et al. Arginine methylation analysis of the splicing-associated SR protein SFRS9/SRP30C. Cell Mol Biol Lett 14, 657-669, doi:10.2478/s11658-009-0024-2 (2009)).

FIG. 3: Methylscan evaluation of cell lines treated with Compound D. Percent of proteins with methylation changes (independent of directionality of change) are categorized by functional group as indicated.

FIG. 4: Mode of inhibition against PRMT1 by Compound A. IC₅₀ values were determined following a 18 minute PRMT1 reaction and fitting the data to a 3-parameter dose-response equation. (A) Representative experiment showing Compound A IC₅₀ values plotted as a function of [SAM]% Kₘ^{app} fit to an equation for uncompetitive inhibition IC₅₀=Kᵢ/(1+(Kₘ^{app} [S])). (B) Representative experiment showing IC₅₀ values plotted as a function of [Peptide]/Kₘ^{app}. Inset shows data fit to an equation for mixed inhibition to evaluate Compound A inhibition of PRMT1 with respect to peptide H4 1-21 substrate (v=Vₘₐₓ*[S]/(Kₘ*(1+[I]/Kᵢ)+[S]*(1+[I]/K′))). An alpha value (α=Kᵢ′/Kᵢ)>0.1 but <10 is indicative of a mixed inhibitor.

FIG. 5: Potency of Compound A against PRMT1. PRMT1 activity was monitored using a radioactive assay run under balanced conditions (substrate concentrations equal to Kₘ^{app}) measuring transfer of ³H from SAM to a H4 1-21 peptide. IC₅₀ values were determined by fitting the data to a 3-parameter dose-response equation. (A) IC₅₀ values plotted as a function of PRMT1:SAM:Compound A-tri-HCl preincubation time. Open and filled circles represent two independent experiments (0.5 nM PRMT1). Inset shows a representative IC₅₀ curve for Compound A-tri-HCl inhibition of PRMT1 activity following a 60 minute PRMT1:SAM:Compound A-tri-HCl preincubation. (B) Compound A inhibition of PRMT1 categorized by salt form. IC₅₀ values were determined following a 60 minute PRMT1:SAM:Compound A preincubation and a 20 minute reaction.

FIG. 6: The crystal structure resolved at 2.48 Å for PRMT1 in complex with Compound A (orange) and SAH (purple). The inset reveals that the compound is bound in the peptide binding pocket and makes key interactions with PRMT1 sidechains.

FIG. 7: Inhibition of PRMT1 orthologs by Compound A. PRMT1 activity was monitored using a radioactive assay run under balanced conditions (substrate concentrations equal to Kₘ^{app}) measuring transfer of ³H from SAM to a H4 1-21 peptide. IC₅₀ values were determined by fitting the data to a 3-parameter dose-response equation. (A) IC₅₀ values plotted as a function of PRMT1:SAM:Compound A preincubation time for rat (◯) and dog (●) orthologs. (B) IC₅₀ values plotted as a function of rat (◯), dog (●) or human (□) PRMT1 concentration. (C) IC₅₀ values were determined following a 60 minute PRMT1:SAM:Compound A preincubation and a 20 minute reaction. Data is an average from testing multiple salt forms of Compound A. Kᵢ^{*app} values were calculated based on the equation Kᵢ=IC₅₀/(1+(Kₘ/[S])) for an uncompetitive inhibitor and the assumption that the IC₅₀ determination was representative of the ESI* conformation.

FIG. 8: Potency of Compound A against PRMT family members. PRMT activity was monitored using a radioactive assay run under balanced conditions (substrate concentrations at Kₘ^{app}) following a 60 minute PRMT:SAM:Compound A preincubation. IC₅₀ values for Compound A were determined by fitting data to a 3-parameter dose-response equation. (A) Data is an average from testing multiple salt forms of Compound A. Kᵢ^{*app} value were calculated based on the equation Kᵢ=IC₅₀/(1+(Kₘ/[S])) for an uncompetitive inhibitor and the assumption that the IC₅₀ determination was representative of the ESI* conformation. (B) IC₅₀ values plotted as a function of PRMT3 (●), PRMT4 (◯), PRMT6 (▪) or PRMT8 (□):SAM:Compound A preincubation time.

FIG. 9: MMA in-cell-western. RKO cells were treated with Compound A-tri-HCl (“Compound A-A”), Compound A-mono-HCl (“Compound A-B”), Compound A-free-base (“Compound A-C”), and Compound A-di-HCl (“Compound A-D”) for 72 hours. Cells were fixed, stained with anti-Rme1 GG to detect MMA and anti-tubulin to normalize signal, and imaged using the Odyssey imaging system. MMA relative to tubulin was plotted against compound concentration to generate a curve fit (A) in GraphPad using a biphasic curve fit equation. Summary of EC₅₀ (first inflection), standard deviation, and N are shown in (B).

FIG. 10: PRMT1 expression in tumors. mRNA expression levels were obtained from cBioPortal for Cancer Genomics. ACTB levels and TYR are shown to indicate expression of level corresponding to a gene that is ubiquitously expressed versus one that has restricted expression, respectively.

FIG. 11: Antiproliferative activity of Compound A in cell culture. 196 human cancer cell lines were evaluated for sensitivity to Compound A in a 6-day growth assay. gIC₅₀ values for each cell line are shown as bar graphs with predicted human exposure as indicated in (A). Yₘᵢₙ−T₀, a measure of cytotoxicity, is plotted as a bar-graph in (B), in which gIC₁₀₀ values for each cell line are shown as red dots. The Cₐᵥₑ calculated from the rat 14-day MTD (150 mg/kg, Cₐᵥₑ=2.1 μM) is indicated as a red dashed line.

FIG. 12: Timecourse of Compound A effects on arginine methylation marks in cultured cells. (A) Changes in ADMA, SDMA, and MMA in Toledo DLBCL cells treated with Compound A. Changes in methylation are shown normalized relative to tubulin±SEM (n=3). (B) Representative western blots of arginine methylation marks. Regions quantified are denoted by black bars on the right of the gel.

FIG. 13: Dose response of Compound A on arginine methylation. (A) Representative western blot images of MMA and ADMA from the Compound A dose response in the U2932 cell line. Regions quantified for (B) are denoted by black bars to the left of gels. (B) Minimal effective Compound A concentration required for 50% of maximal induction of MMA or 50% maximal reduction ADMA in 5 lymphoma cell lines after 72 hours of exposure±standard deviation (n=2). Corresponding gIC₅₀ values in 6-day growth death assay are as indicated in red.

FIG. 14: Durability of arginine methylation marks in response to Compound A in lymphoma cells. (A) Stability of changes to ADMA. SDMA, and MMA in the Toledo DLBCL cell line cultured with Compound A. Changes in methylation are shown normalized relative to tubulin±SEM (n=3). (B) Representative western blots of arginine methylation marks. Regions quantified for (A) are denoted by black bars on the side of the gel.

FIG. 15: Proliferation timecourse of lymphoma cell lines. Cell growth was assessed over a 10-day timecourse in the Toledo (A) and Daudi (B) cell lines (n=2 per cell line). Representative data for a single biological replicate are shown.

FIG. 16: Anti-proliferative effects of Compound A in lymphoma cell lines at 6 and 10 days. (A) Average gIC₅₀ values from 6 day (light blue) and 10 day (dark blue) proliferation assays in lymphoma cell lines. (B) Yₘᵢₙ-T₀ at 6 day (light blue) and 10 day (dark blue) with corresponding gIC₁₀₀ (red points).

FIG. 17: Anti-proliferative effects of Compound A in lymphoma cell lines as classified by subtype. (A) gIC₅₀ values for each cell line are shown as bar graphs. Yₘᵢₙ-T₀, a measure of cytotoxicity, is plotted as a bar-graph in (B), in which gIC₁₀₀ values for each cell line are shown as red dots. Subtype information was collected from the ATCC or DSMZ cell line repositories.

FIG. 18: Propidium iodide FACS analysis of cell cycle in human lymphoma cell lines. Three lymphoma cell lines, Toledo (A), U2932 (B), and OCI-Ly1 (C) were treated with 0, 1, 10, 100, 1000, and 10,000 nM Compound A for 10 days with samples taken on days 3, 5, 7, 10 post treatment. Data represents the average±SEM of biological replicates, n=2.

FIG. 19: Caspase-317 activation in lymphoma cell lines treated with Compound A. Apoptosis was assessed over a 10-day timecourse in the Toledo (A) and Daudi (B) cell lines. Caspase 3/7 activation is shown as fold-induction relative to DMSO-treated cells. Two independent replicates were performed for each cell line. Representative data are shown for each.

FIG. 20: Efficacy of Compound A in mice bearing Toledo xenografts. Mice were treated QD (37.5, 75, 150, 300, 450, or 600 mg/kg) with Compound A orally or BID with 75 mg/kg (B) over a period of 28 (A) or 24 (B) days and tumor volume was measured twice weekly.

FIG. 21: Effect of Compound A in AML cell lines at 6 and 10 Days. (A) Average gIC₅₀ values from 6 day (light blue) and 10 day (dark blue) proliferation assays in AML cell lines. (B) Yₘᵢₙ-T₀ at 6 day (light blue) and 10 day (dark blue) with corresponding gIC₁₀₀ (red points).

FIG. 22: In vitro proliferation timecourse of ccRCC cines with Compound A. (A) Growth relative to control (DMSO) for 2 ccRCC cell lines. Representative curves from a single replicate are shown. (B) Summary of gIC₅₀ and % growth inhibition for ccRCC cell lines during the timecourse (Average±SD; n=2 for each line).

FIG. 23: Efficacy of Compound A in ACHN xenografts. Mice were treated daily with Compound A orally over a period of 28 days and tumor volume was measured twice weekly.

FIG. 24: Anti-proliferative effects of Compound A in breast cancer cell lines. Bar graphs of gIC₅₀ and growth inhibition (%) (red circles) for breast cancer cell lines profiled with Compound A in the 6-day proliferation assay. Cell lines representing triple negative breast cancer (TNBC) are shown in orange; other subtypes are in blue.

FIG. 25: Effect of Compound A in Breast Cancer Cell Lines at 7 and 12 Days. Average growth inhibition (%) values from 7 day (light blue) and 10 day (dark blue) proliferation assays in breast cancer cell lines with corresponding gIC₅₀ (red points). The increase in potency and percent inhibition observed in long-term proliferation assays with breast cancer, but not lymphoma or AML cell lines, suggest that certain tumor types require a longer exposure to Compound A to fully reveal anti-proliferative activity.

FIG. 26: Combination with immunotherapy. Average tumor volume (A) and survival (B) for single agent and combination in the syngeneic CloudmanS91 tumor model. (C) Individual tumor growth for animals in each arm of the efficacy study.

FIG. 27: Compound A treatment of CloudmanS91 cells in culture. Cells were treated in 6-day proliferation assay in 96-well format and gIC₅₀=9515±231.8 nM was determined.

FIG. 28: Alignment of the amino acid sequences of 106-222, humanized 106-222 (Hu106), and human acceptor X61012 (GenBank accession number) VH sequences.

FIG. 29: Alignment of the amino acid sequences of 106-222, humanized 106-222 (Hu106), and human acceptor AJ388641 (GenBank accession number) VL sequences.

FIG. 30: Nucleotide sequence of the Hu106 VH gene flanked by SpeI and HindIII sites with the deduced amino acid sequence.

FIG. 31: Nucleotide sequence of the Hu106-222 VL gene flanked by NheI and EcoRI sites with the deduced amino acid sequence.

FIG. 32: Alignment of the amino acid sequences of 119-122, humanized 119-122 (Hu119), and human acceptor Z14189 (GenBank accession number) VH sequences.

FIG. 33: Alignment of the amino acid sequences of 119-122, humanized 119-122 (Hu119), and human acceptor M29469 (GenBank accession number) VL sequences.

FIG. 34: Nucleotide sequence of the Hu119 VH gene flanked by SpeI and HindIII sites with the deduced amino acid sequence.

FIG. 35: Nucleotide sequence of the Hu119 VL gene flanked by NheI and EcoRI sites with the deduced amino acid sequence.

FIG. 36: Nucleotide sequence of mouse 119-43-1 VH cDNA with the deduced amino acid sequence.

FIG. 37: Nucleotide sequence of mouse 119-43-1 VL cDNA and the deduced amino acid sequence.

FIG. 38: Nucleotide sequence of the designed 119-43-1 VH gene flanked by SpeI and HindIII sites with the deduced amino acid sequence.

FIG. 39: Nucleotide sequence of the designed 119-43-1 VL gene flanked by NheI and EcoRI sites with the deduced amino acid sequence.

FIG. 40: Combination with immunotherapy. Average survival for single agent and combination in the A20 tumor model.

FIG. 41: Combination with immunotherapy. Average survival for single agent and combination in the CT26 tumor model.

### SUMMARY OF THE INVENTION

In one embodiment the present invention provides a combination of a Type I protein arginine methyltransferase (Type I PRMT) inhibitor and an immuno-modulatory agent selected from: an anti-PD-1 antibody or antigen binding fragment thereof, an anti-PDL1 antibody or antigen binding fragment thereof, and an anti-OX40 antibody or antigen binding fragment thereof.

In one embodiment, methods are provided for treating cancer in a human in need thereof, the methods comprising administering to the human a combination of a Type I protein arginine methyltransferase (Type I PRMT) inhibitor and an immuno-modulatory agent selected from: an anti-PD-1 antibody or antigen binding fragment thereof, an anti-PDL1 antibody or antigen binding fragment thereof, and an anti-OX40 antibody or antigen binding fragment thereof, together with at least one of: a pharmaceutically acceptable carrier and a pharmaceutically acceptable diluent, thereby treating the cancer in the human.

In one embodiment, the present invention provides a pharmaceutical composition comprising a therapeutically effective amount of a Type I protein arginine methyltransferase (Type I PRMT) inhibitor and a second pharmaceutical composition comprising a therapeutically effective amount of an immuno-modulatory agent selected from: an anti-PD-1 antibody or antigen binding fragment thereof, an anti-PDL1 antibody or antigen binding fragment thereof, and an anti-OX40 antibody or antigen binding fragment thereof.

In one embodiment, methods are provided for treating cancer in a human in need thereof, the methods comprising administering to the human a therapeutically effective amount of a pharmaceutical composition comprising a Type I protein arginine methyltransferase (Type I PRMT) inhibitor and a pharmaceutical composition comprising an immuno-modulatory agent selected from: an anti-PD-1 antibody or antigen binding fragment thereof, an anti-PDL1 antibody or antigen binding fragment thereof, and an anti-OX40 antibody or antigen binding fragment thereof, thereby treating the cancer in the human.

In one embodiment, the present invention provides use of a combination of a Type I protein arginine methyltransferase (Type I PRMT) inhibitor and an immuno-modulatory agent selected from: an anti-PD-1 antibody or antigen binding fragment thereof, an anti-PDL1 antibody or antigen binding fragment thereof, and an anti-OX40 antibody or antigen binding fragment thereof, for the manufacture of a medicament.

In one embodiment, the present invention provides use of a combination of a Type I protein arginine methyltransferase (Type I PRMT) inhibitor and an immuno-modulatory agent selected from: an anti-PD-1 antibody or antigen binding fragment thereof, an anti-PDL1 antibody or antigen binding fragment thereof, and an anti-OX40 antibody or antigen binding fragment thereof, for the treatment of cancer.

### DETAILED DESCRIPTION OF THE INVENTION

### Definitions

As used herein “Type I protein arginine methyltransferase inhibitor” or “Type I PRMT inhibitor” means an agent that inhibits any one or more of the following: protein arginine methyltransferase 1 (PRMT1), protein arginine methyltransferase 3 (PRMT3), protein arginine methyltransferase 4 (PRMT4), protein arginine methyltransferase 6 (PRMT6) inhibitor, and protein arginine methyltransferase 8 (PRMT8). In some embodiments, the Type I PRMT inhibitor is a small molecule compound. In some embodiments, the Type I PRMT inhibitor selectively inhibits any one or more of the following: protein arginine methyltransferase 1 (PRMT1), protein arginine methyltransferase 3 (PRMT3), protein arginine methyltransferase 4 (PRMT4), protein arginine methyltransferase 6 (PRMT6) inhibitor, and protein arginine methyltransferase 8 (PRMT8). In some embodiments, the Type I PRMT inhibitor is a selective inhibitor of PRMT1, PRMT3, PRMT4, PRMT6, and PRMT8.

Arginine methyltransferases are attractive targets for modulation given their role in the regulation of diverse biological processes. It has now been found that compounds described herein, and pharmaceutically acceptable salts and compositions thereof, are effective as inhibitors of arginine methyltransferases.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et ah. Enantiomers, Racemates and Resolutions (Wiley Interscience, New York, 1981); Wilen et ah, Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

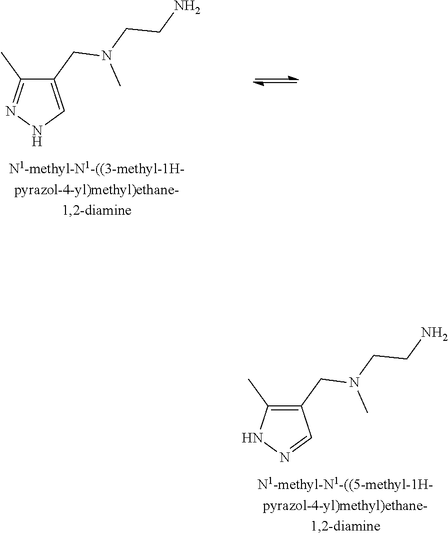

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with is ¹⁸F. or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and subrange within the range. For example “C₁₋₆ alkyl” is intended to encompass, C₁; C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Radical” refers to a point of attachment on a particular group. Radical includes divalent radicals of a particular group.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted. e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms and one or more carbon-carbon double bonds (e.g., 1, 2, 3, or 4 double bonds), and optionally one or more triple bonds (e.g., 1, 2, 3, or 4 triple bonds) (“C₂₋₂₀ alkenyl”). In certain embodiments, alkenyl does not comprise triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”) In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms and one or more carbon-carbon triple bonds (e.g., 1, 2, 3, or 4 triple bonds), and optionally one or more double bonds (e.g., 1, 2, 3, or 4 double bonds) (“C₂₋₂₀ alkynyl”). In certain embodiments, alkynyl does not comprise double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₉ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Fused” or “ortho-fused” are used interchangeably herein, and refer to two rings that have two atoms and one bond in common, e.g.,

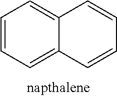

“Bridged” refers to a ring system containing (1) a bridgehead atom or group of atoms which connect two or more non-adjacent positions of the same ring; or (2) a bridgehead atom or group of atoms which connect two or more positions of different rings of a ring system and does not thereby form an ortho-fused ring, e.g.,

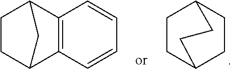 [SMILES: c1ccc2c(c1)C1CCC2C1 ; C1CC2CCC1CC2]

“Spiro” or “Spiro-fused” refers to a group of atoms which connect to the same atom of a carbocyclic or heterocyclic ring system (geminal attachment), thereby forming a ring, e.g.,

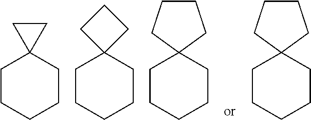

Spiro-fusion at a bridgehead atom is also contemplated.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group refers to a radical of a non-aromatic cyclic hydrocarbon group having from 3 to 10 ring carbon atoms (C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or is a fused, bridged or spiro-fused ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In certain embodiments, heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms independently selected from nitrogen, oxygen, and sulfur. In some embodiments, the 5-6 membered heterocyclyl has 1-2 ring heteroatoms independently selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxepanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 It electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted. e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-14 membered monocyclic or polycyclic (e.g., bicyclic or tricyclic) 4n+2 aromatic ring system (e.g., having 6 or 10 it electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, heteroaryl refers to a radical of a 5-10 membered monocyclic or bicyclic 4n+2 aromatic ring system having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-14 membered aromatic ring system having ring carbon atoms and 1-4 ring heteroatoms provided in the aromatic ring system, wherein each heteroatom is independently selected from nitrogen, oxygen, and sulfur (“5-14 membered heteroaryl”). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms independently selected from nitrogen, oxygen, and sulfur. In some embodiments, the 5-6 membered heteroaryl has 1-2 ring heteroatoms independently selected from nitrogen, oxygen, and sulfur. In some embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary 5,6-bicyclic heteroaryl groups include, without limitation, any one of the following formulae:

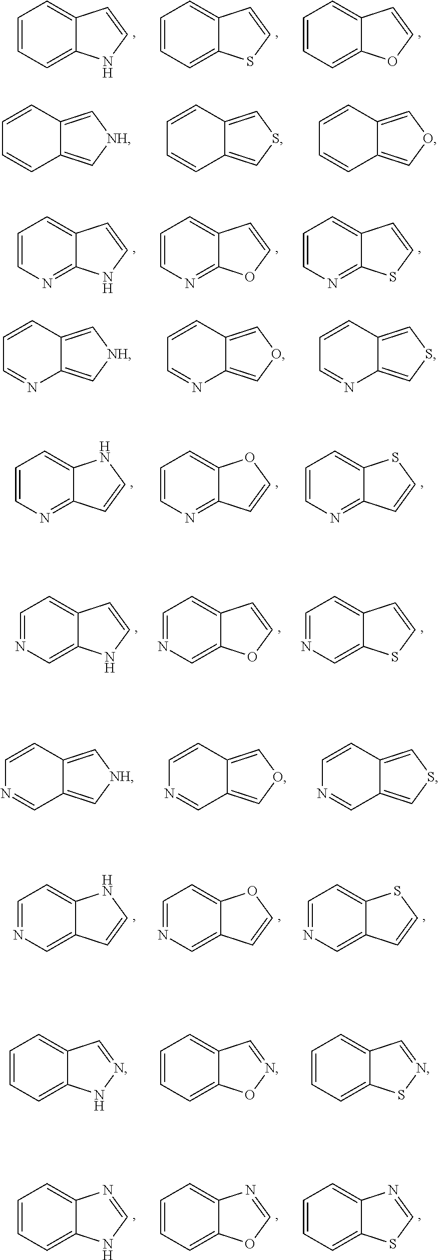

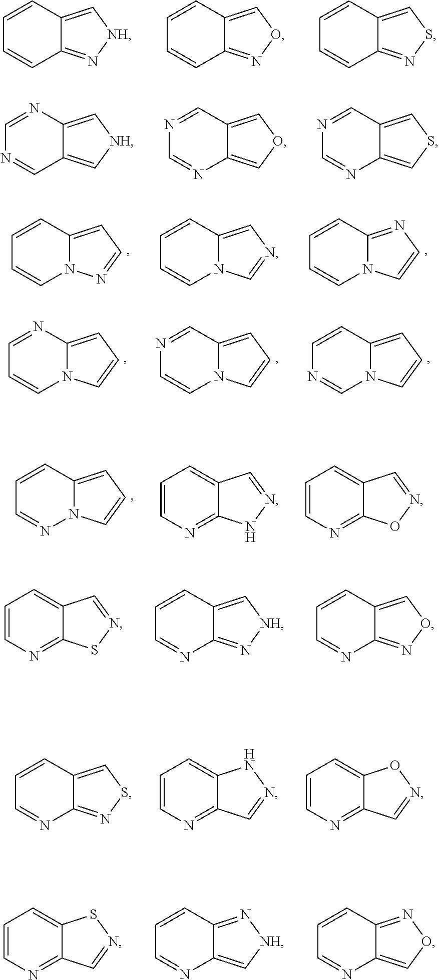

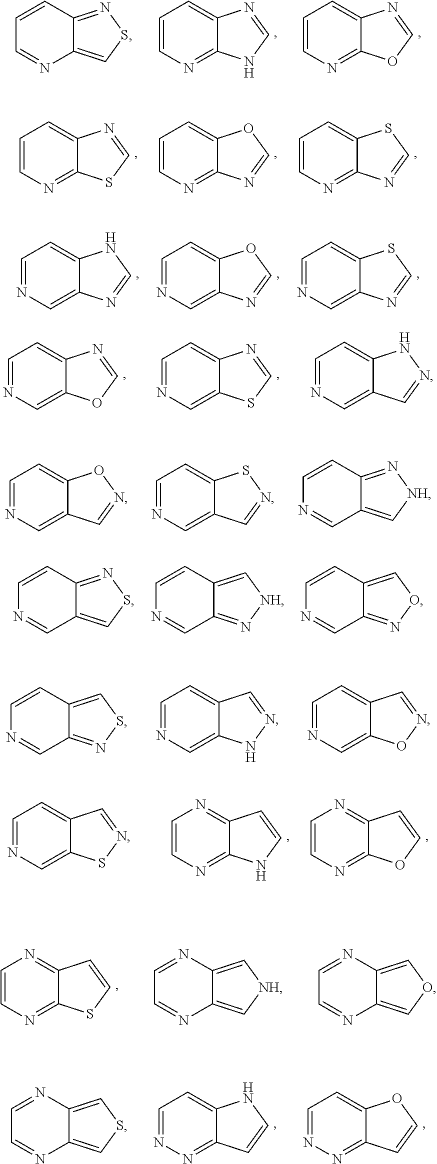

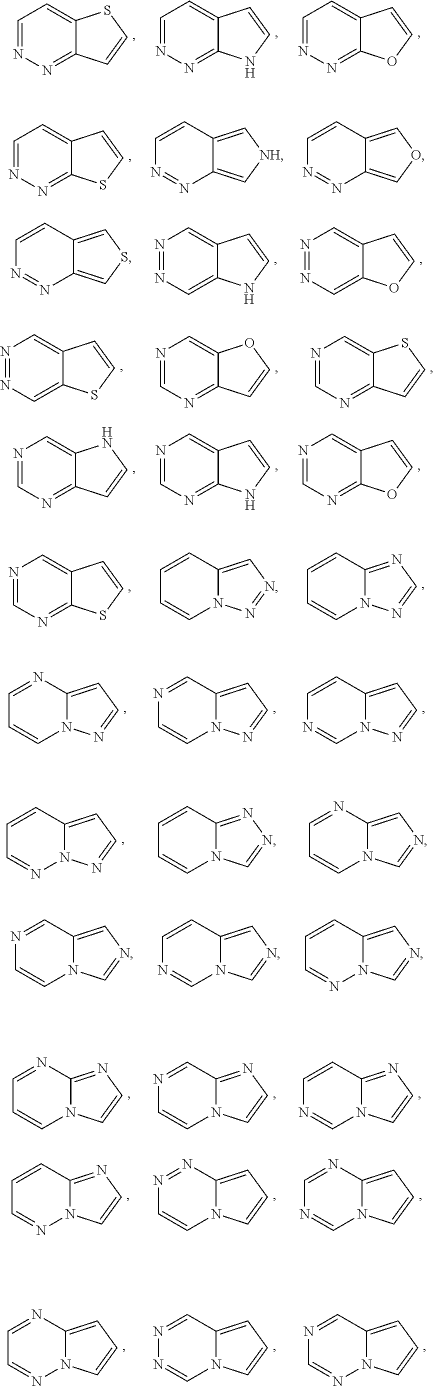

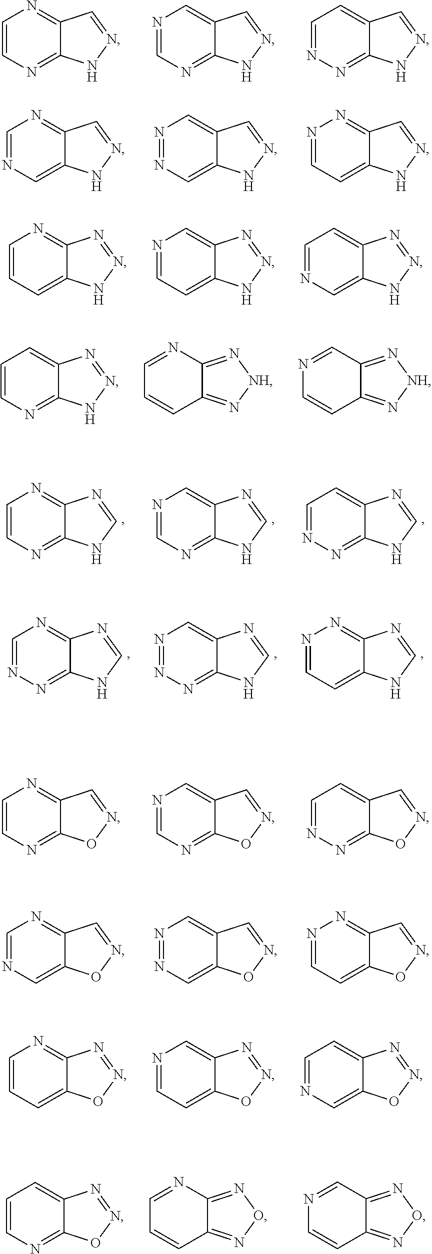

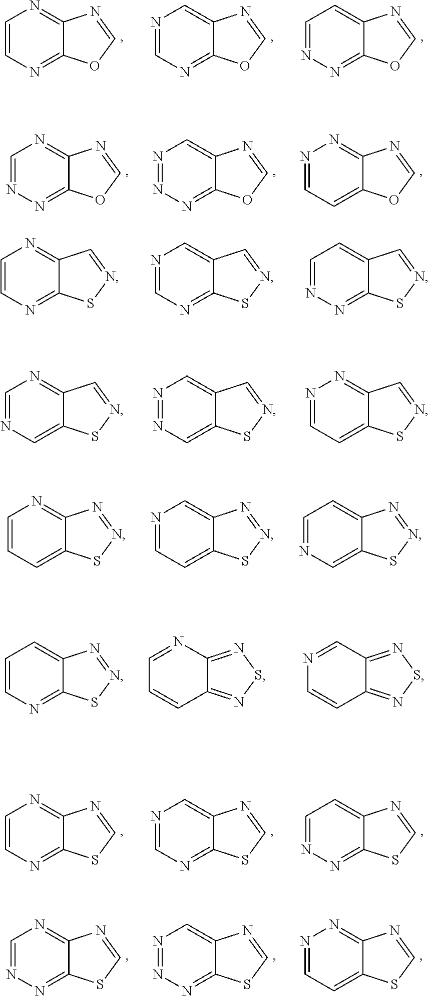

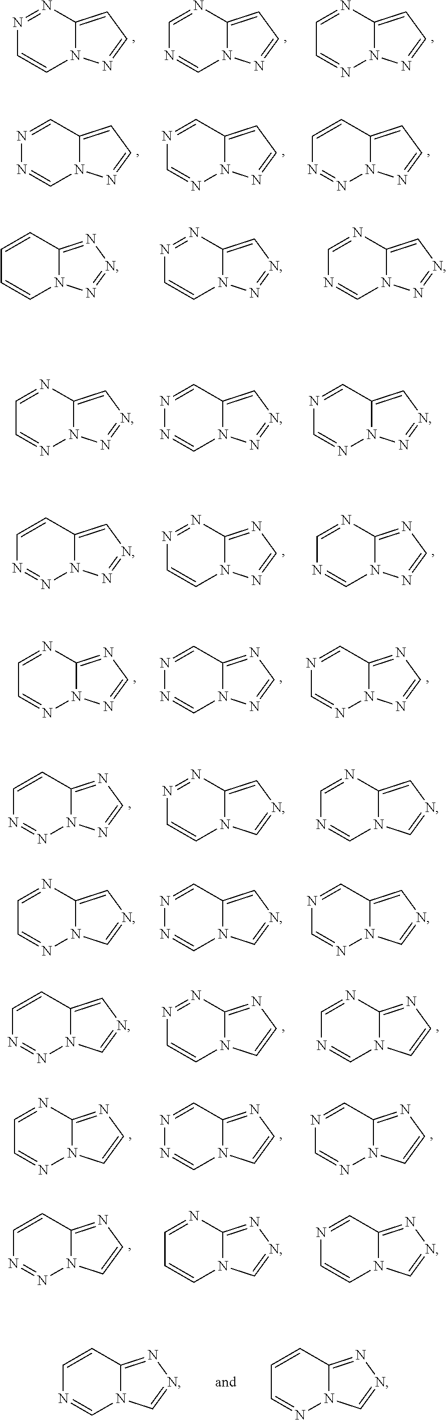

In any of the monocyclic or bicyclic heteroaryl groups, the point of attachment can be any carbon or nitrogen atom, as valency permits.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, alkyl, alkenyl, alkynyl, carbocyclyl, heterocyclyl, aryl, and heteroaryl groups, as defined herein, are optionally substituted (e.g., “substituted” or “unsubstituted” aliphatic, “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₃H, —SO₃H, —OH, —OR^{aa}, —ON(R^{bb})₂, —N(R^{bb})₂, —N(R^{bb})₃, ⁺X, —N(OR^{cc})R^{bb}, —SH, —SR^{aa}, —SSR^{CC}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O))(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{CC})₂, —P(R^{CC})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O))R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{CC})₂, —CN, —C(═O)R^{aa}, —C(═O)N(R^{cc})₂, —CO₂R^{aa}, —SO₂R^{aa}, —C(═NR^{cc})OR^{aa}, —C(═NR^{CC})N(R^{CC})₂, —SO₂N(R^{cc})₂, —SO₂R^{cc}, —SO₂OR^{cc}, —SOR^{aa}, —C(═S)N(R^{CC})₂, —C(═O)SR^{cc}, —C(═S)SR^{CC}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl. C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups:

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff} C (═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)S^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₄, perhaloalkyl, C₂₋₆ alkenyl, C₂₋₆ alkynyl, C₃₋₁₀ carbocyclyl, 3-10 membered heterocyclyl. Chao aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₁₋₆ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —O₁₋₆ alkyl, —ON(C₁₋₆ alkyl)₂, —N(C₁₋₆ alkyl)₂, —N(C₁₋₆alkyl)₃ ⁺X, —NH(C₁₋₆ alkyl)₂⁺X⁻, —NH₂(C₁₋₆ alkyl) ⁺X, —NH₃ ⁺X, —N(OC₁₋₆ alkyl)(C₁₋₆ alkyl), —N(OH)(C₁₋₆ alkyl), —NH(OH), —SH, —S₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, SO₂ C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate. 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{CC})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{CC})N(R^{CC})₂, —SO₂N(R^{cc})₂, —SO₂R^{cc}, —SO₂OR^{cc}, —SOR^{aa}, —C(═S)N(R^{CC})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{CC})₂, —C(═O)R^{aa}, —C(═O))N(R^{cc})₂, —CO₂R^{aa}, —SO₂R^{aa}, —C(═NR^{cc})R^{aa}, —C(═NR^{cc})OR^{aa}, —C(═NR^{CC})N(R^{CC})₂, —SO₂N(R^{cc})₂, —SO₂R^{cc}, —SO₂OR^{cc}, —SOR^{aa}, —C(═S)N(R^{CC})₂, —C(═O)SR^{cc}, —C(═S)SR^{cc}, C₁₋₁₀ alkyl ≡e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3 rd edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-{p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10, 10,10,10-tetrahydrothioxanthyl)] methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(l-adamantyl)-1-methylethyl carbamate (Adpoc), 1,1-dimethyl-2-haloethyl carbamate, 1,1-dimethyl-2,2-dibromoethyl carbamate (DB-t-BOC). 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc). 2-(2′- and 4′-pyridyl)ethyl carbamate (Pyoc), 2-{N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)] methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxy phenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5, 6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds). 2,2,5,7, 8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl] amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl] amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —C O₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O))R^{aa}, —SO₂ R^{aa}, —Si(R^{aa})₃, —P(R^{CC})₂, —P(R^{CC})₃, —P(═O)₂R^{aa}, —P(═O)(R^{aa})₂, —P(═O)(OR^{cc})₂, —P(O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3 edition. John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-111)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsiyl (IPDMS), diethylisopropylsilyl (DEIPS), dimethylthexylsilyl, t-butyldimethylsilyl (TBDMS), t-butyldiphenylsilyl (TBDPS), tribenzylsilyl, trip-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate. 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate, alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂ R^{aa}, —Si(R^{aa})₃ —P(R^{CC})₂, —P(R^{CC})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts. 3^{rd} edition, John Wiley & Sons, 1999, incorporated herein by reference.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66: 1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate. 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The present invention provides Type I PRMT inhibitors. In one embodiment, the Type I PRMT inhibitor is a compound of Formula (I):

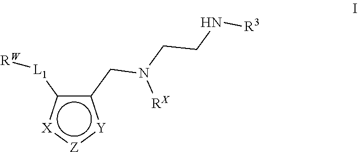

or a pharmaceutically acceptable salt thereof,

wherein

X is N, Z is NR⁴, and Y is CR⁵; or

X is NR⁴, Z is N, and Y is CR⁵; or

X is CR⁵, Z is NR⁴, and Y is N; or

X is CR⁵, Z is N, and Y is NR⁴;

R^{X} is optionally substituted C₁₋₄ alkyl or optionally substituted C₃₋₄ cycloalkyl;

L₁ is a bond, —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—, —C(═NOR^{A})—, —C(═NR^{B})N(R^{B})—, —NR^{B}C(═NR^{B})—, —C(S)—, —C(S)N(R^{B})—, —NR^{B}C(S)—, —S(O)—, —OS(O)₂—, —S(O)₂O—, —SO₂—, —N(R^{B})SO₂—, —SO₂N(R^{B})—, or an optionally substituted C₁₋₆ saturated or unsaturated hydrocarbon chain, wherein one or more methylene units of the hydrocarbon chain is optionally and independently replaced with —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—, —C(═NOR^{A})—, —C(═NR^{B})N(R^{B})—, —NR^{B}C(═NR^{B})—, —C(S)—, —C(S)N(R^{B})—, —NR^{B}C(S)—, —S(O)—, —OS(O)₂—, —S(O)₂O—, —SO₂—, —N(R^{B})SO₂—, or —SO—N(R^{B})—;

each R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom;

each R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or an R^{B} and R^{W} on the same nitrogen atom may be taken together with the intervening nitrogen to form an optionally substituted heterocyclic ring;

R^{W} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl; provided that when L₁ is a bond, R^{W} is not hydrogen, optionally substituted aryl, or optionally substituted heteroaryl;

R³ is hydrogen, C₁₋₄ alkyl, or C₃₋₄ cycloalkyl;

R⁴ is hydrogen, optionally substituted C₁₋₆alkyl, optionally substituted C₂₋₆ alkenyl, optionally substituted C₂₋₆ alkynyl, optionally substituted C₃₋₇ cycloalkyl, optionally substituted 4- to 7-membered heterocyclyl; or optionally substituted C₁₋₄ alkyl-Cy:

Cy is optionally substituted C₃₋₇ cycloalkyl, optionally substituted 4- to 7-membered heterocyclyl, optionally substituted aryl, or optionally substituted heteroaryl; and

R⁵ is hydrogen, halo, —CN, optionally substituted C₁₋₄ alkyl, or optionally substituted C₃₋₄ cycloalkyl. In one aspect, R³ is a C₁₋₄ alkyl. In one aspect, R³ is methyl. In one aspect, R⁴ is hydrogen. In one aspect, R⁵ is hydrogen. In one aspect, L₁ is a bond.

In one embodiment, the Type I PRMT inhibitor is a compound of Formula (I) wherein -L₁-R^{W} is optionally substituted carbocyclyl.

In one embodiment, the Type I PRMT inhibitor is a compound of Formula (V)

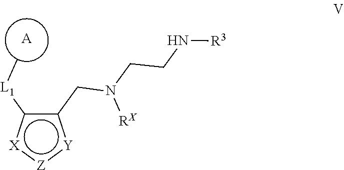

or a pharmaceutically acceptable salt thereof, wherein Ring A is optionally substituted carbocyclyl, optionally substituted heterocyclyl, optionally substituted aryl, or optionally substituted heteroaryl. In one aspect. Ring A is optionally substituted carbocyclyl. In one aspect, R³ is a C₁₋₄ alkyl. In one aspect, R³ is methyl. In one aspect. R^{x} is unsubstituted C₁₋₄ alkyl. In one aspect. R^{x} is methyl. In one aspect, L₁ is a bond.

In one embodiment, the Type I PRMT inhibitor is a compound of Formula (VI)

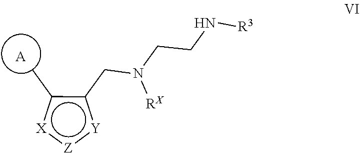

or a pharmaceutically acceptable salt thereof. In one aspect, Ring A is optionally substituted carbocyclyl. In one aspect. R³ is a C₁₋₄ alkyl. In one aspect. R³ is methyl. In one aspect, R^{x} is unsubstituted C₁₋₄ alkyl. In one aspect, R^{x} is methyl.

In one embodiment, the Type I PRMT inhibitor is a compound of Formula (II):

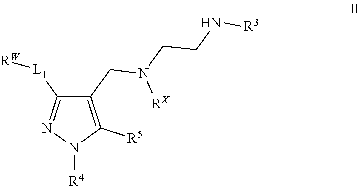

or a pharmaceutically acceptable salt thereof. In one aspect, -L₁-R^{W} is optionally substituted carbocyclyl. In one aspect. R³ is a C₁₋₄ alkyl. In one aspect, R³ is methyl. In one aspect, R^{x} is unsubstituted C₁₋₄alkyl. In one aspect, R^{x} is methyl. In one aspect, R⁴ is hydrogen.

In one embodiment, the Type I PRMT inhibitor is Compound A:

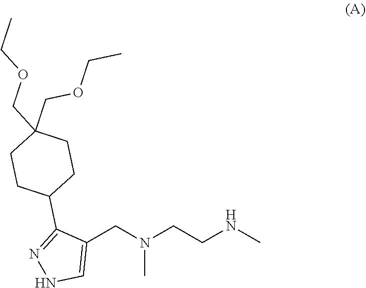

or a pharmaceutically acceptable salt thereof. Compound A and methods of making Compound A are disclosed in PCT/US2014/029710, in at least page 171 (Compound 158) and page 266, paragraph [00331].

In one embodiment, the Type I PRMT inhibitor is Compound A-tri-HCl, a tri-HCl salt form of Compound A. In another embodiment, the Type I PRMT inhibitor is Compound A-mono-HCl, a mono-HCl salt form of Compound A. In yet another embodiment, the Type I PRMT inhibitor is Compound A-free-base, a free base form of Compound A. In still another embodiment, the Type I PRMT inhibitor is Compound A-di-HCl, a di-HCl salt form of Compound A.

In one embodiment, the Type I PRMT inhibitor is Compound D:

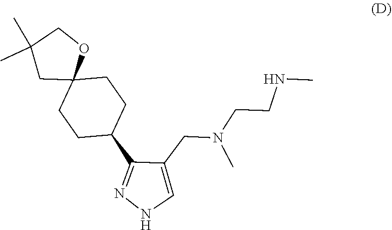

or a pharmaceutically acceptable salt thereof.

Type I PRMT inhibitors are further disclosed in PCT/US2014/029710, which is incorporated herein by reference. Exemplary Type I PRMT inhibitors are disclosed in Table IA and Table 1B of PCT/US2014/029710, and methods of making the Type I PRMT inhibitors are described in at least page 226, paragraph [00274] to page 328, paragraph of PCT/US2014/029710. “Antigen Binding Protein (ABP)” means a protein that binds an antigen, including antibodies or engineered molecules that function in similar ways to antibodies. Such alternative antibody formats include triabody, tetrabody, miniantibody, and a minibody, Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP may comprise the VH regions of the invention formatted into a full length antibody, a (Fab′)2 fragment, a Fab fragment, a bi-specific or biparatopic molecule or equivalent thereof (such as scFV, bi- tri- or tetra-bodies. Tandabs, etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533, which comprises an antigen binding region and a non-immunoglobulin region. The terms “ABP,” “antigen binding protein,” and “binding protein” are used interchangeably herein.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra: Okazaki et al. (2002) Curr. Opin. Immunol 14:391779-82; Bennett et al. (2003) J Immunol 170:711-8) The initial members of the family, CD28 and ICOS, were discovered by functional effects on augmenting T cell proliferation following the addition of monoclonal antibodies (Hutloff et al. (1999) Nature 397:263-266; Hansen et al. (1980) Immunogenics 10:247-260). PD-1 was discovered through screening for differential expression in apototic cells (Ishida et al. (1992) EMBO J 11:3887-95) The other members of the family, CTLA-4, and BTLA were discovered through screening for differential expression in cytotoxic T lymphocytes and TH1 cells, respectively. CD28, ICOS and CTLA-4 all have an unpaired cysteine residue allowing for homodimerization. In contrast, PD-1 is suggested to exist as a monomer, lacking the unpaired cysteine residue characteristic in other CD28 family members. PD-1 antibodies and methods of using in treatment of disease are described in U.S. Pat. Nos. 7,595,048; 8,168,179; 8,728,474; 7,722,868; 8,008,449; 7,488,802; 7,521,051; 8,088,905; 8,168,757; 8,354,509; and US Publication Nos. US20110171220; US20110171215; and US20110271358. Combinations of CTLA-4 and PD-1 antibodies are described in U.S. Pat. No. 9,084,776.

As used herein, “PD-1 antagonist” means any chemical compound or biological molecule that blocks binding of PD-L1 expressed on a cancer cell to PD-1 expressed on an immune cell (T cell, B cell or NIT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4. CD274 and B7-H for PD-L1; and PDCD1L2, PDL2, B7-DC, Btdc and CD273 for PD-L2. Human PD-1 amino acid sequences can be found in NCBI Locus No.: NP_005009. Human PD-L1 and PD-L2 amino acid sequences can be found in NCBI Locus No.: NP 054862 and NP 079515, respectively.

PD-1 antagonists useful in the any of the aspects of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the various aspects and embodiments of the present invention, are described in U.S. Pat. Nos. 8,552,154; 8,354,509; 8,168,757; 8,008,449; 7,521,051; 7,488,802; WO2004072286; WO2004056875; and WO2004004771.

Other PD-1 antagonists useful in the any of the aspects and embodiments of the present invention include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010027827 and WO2011066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

Nivolumab is a humanized monoclonal anti-PD-1 antibody commercially available as OPDIVO®. Nivolumab is indicated for the treatment of some unresectable or metastatic melanomas. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

Pembrolizumab is a humanized monoclonal anti-PD-1 antibody commercially available as KEYTRUDA®. Pembrolizumab is indicated for the treatment of some unresectable or metastatic melanomas. The amino acid sequence of pembrolizumab and methods of using are disclosed in U.S. Pat. No. 8,168,757.

PD-L1 is a B7 family member that is expressed on many cell types, including APCs and activated T cells (Yamazaki et al. (2002) J. Immunol. 169:5538). PD-L1 binds to both PD-1 and B7-1. Both binding of T-cell-expressed B7-1 by PD-L1 and binding of T-cell-expressed PD-L1 by B7-1 result in T cell inhibition (Butte et al. (2007) Immunity 27:111). There is also evidence that, like other B7 family members, PD-L1 can also provide costimulatory signals to T cells (Subudhi et al. (2004) J. Clin. Invest. 113:694; Tamura et al. (2001) Blood 97:1809). PD-L1 (human PD-L1 cDNA is composed of the base sequence shown by EMBL/GenBank Acc. No. AF233516 and mouse PD-L1 cDNA is composed of the base sequence shown by NM.sub.-021893) that is a ligand of PD-1 is expressed in so-called antigen-presenting cells such as activated monocytes and dendritic cells (Journal of Experimental Medicine (2000), vol. 19, issue 7, p 1027-1034). These cells present interaction molecules that induce a variety of immuno-inductive signals to T lymphocytes, and PD-L1 is one of these molecules that induce the inhibitory signal by PD-1. It has been revealed that PD-L1 ligand stimulation suppressed the activation (cellular proliferation and induction of various cytokine production) of PD-1 expressing T lymphocytes. PD-L1 expression has been confirmed in not only immunocompetent cells but also a certain kind of tumor cell lines (cell lines derived from monocytic leukemia, cell lines derived from mast cells, cell lines derived from hepatic carcinomas, cell lines derived from neuroblasts, and cell lines derived from breast carcinomas) (Nature Immunology (2001), vol. 2, issue 3, p. 261-267).

Anti-PD-L1 antibodies and methods of making the same are known in the art. Such antibodies to PD-L1 may be polyclonal or monoclonal, and/or recombinant, and/or humanized. PD-L1 antibodies are in development as immuno-modulatory agents for the treatment of cancer.

Exemplary PD-L1 antibodies are disclosed in U.S. Pat. Nos. 9,212,224; 8,779,108; 8,552,154; 8,383,796; 8,217,149; US Patent Publication No. 20110280877; WO2013079174; and WO2013019906. Additional exemplary antibodies to PD-L1 (also referred to as CD274 or B7-H1) and methods for use are disclosed in U.S. Pat. Nos. 8,168,179; 7,943,743; 7,595,048; WO2014055897; WO2013019906; and WO2010077634. Specific anti-human PD-L1 monoclonal antibodies useful as a PD-1 antagonist in the treatment method, medicaments and uses of the present invention include MPDL3280A, BMS-936559, MED14736, MSB0010718C.

Atezolizumab is a fully humanized monoclonal anti-PD-L1 antibody commercially available as TECENTRIQ™. Atezolizumab is indicated for the treatment of some locally advanced or metastatic urothelial carcinomas. Atezolizumab blocks the interaction of PD-L1 with PD-1 and CD80.

CD134, also known as OX40, is a member of the TNFR-superfamily of receptors which is not constitutively expressed on resting naïve T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX40/OX40-ligand (OX40 Receptor)/(OX40L) are a pair of costimulatory molecules critical for T cell proliferation, survival, cytokine production, and memory cell generation. Early in vitro experiments demonstrated that signaling through OX40 on CD4⁺ T cells lead to TH2, but not TH1 development. These results were supported by in vivo studies showing that blocking OX40/OX40 L interaction prevented the induction and maintenance of TH2-mediated allergic immune responses. However, blocking OX40/OX40L interaction ameliorates or prevents TH1-mediated diseases. Furthermore, administration of soluble OX40L or gene transfer of OX40L into tumors were shown to strongly enhance anti-tumor immunity in mice. Recent studies also suggest that OX40/OX40L may play a role in promoting CD8 T cell-mediated immune responses. As discussed herein, OX40 signaling blocks the inhibitory function of CD4⁺ CD25⁺ naturally occurring regulatory T cells and the OX40/OX40L pair plays a critical role in the global regulation of peripheral immunity versus tolerance. OX-40 antibodies, OX-40 fusion proteins and methods of using them are disclosed in U.S. Pat. Nos. 7,504,101; 7,758,852; 7,858,765; 7,550,140; 7,960,515; and 9,006,399 and international publications: WO 2003082919; WO 2003068819; WO 2006063067; WO 2007084559; WO 2008051424; WO2012027328; and WO2013028231.

Herein an antigen binding protein (ABP) of the invention or an anti-OX40 antigen binding protein is one that binds OX40, and in some embodiments, does one or more of the following: modulate signaling through OX40, modulates the function of OX40, agonize OX40 signaling, stimulate OX40 function, or co-stimulate OX40 signaling. Example 1 of U.S. Pat. No. 9,006,399 discloses an OX40 binding assay. One of skill in the art would readily recognize a variety of other well known assays to establish such functions.

In one embodiment, the OX40 antigen binding protein is one disclosed in WO2012/027328 (PCT/US2011/048752), international filing date 23 Aug. 2011. In another embodiment, the antigen binding protein comprises the CDRs of an antibody disclosed in WO2012/027328 (PCT/US2011/048752), international filing date 23 Aug. 2011, or CDRs with 90% identity to the disclosed CDR sequences. In a further embodiment the antigen binding protein comprises a VH, a VL, or both of an antibody disclosed in WO2012/027328 (PCT/US2011/048752), international filing date 23 Aug. 2011, or a VH or a VL with 90% identity to the disclosed VH or VL sequences.

In another embodiment, the OX40 antigen binding protein is disclosed in WO2013/028231 (PCT/US2012/024570), international filing date 9 Feb. 2012. In another embodiment, the antigen binding protein comprises the CDRs of an antibody disclosed in WO2013/028231 (PCT/US2012/024570), international filing date 9 Feb. 2012, or CDRs with 90% identity to the disclosed CDR sequences. In a further embodiment, the antigen binding protein comprises a VH, a VL, or both of an antibody disclosed in WO2013/028231 (PCT/US2012/024570), international filing date 9 Feb. 2012, or a VH or a VL with 90% identity to the disclosed VH or VL sequences.

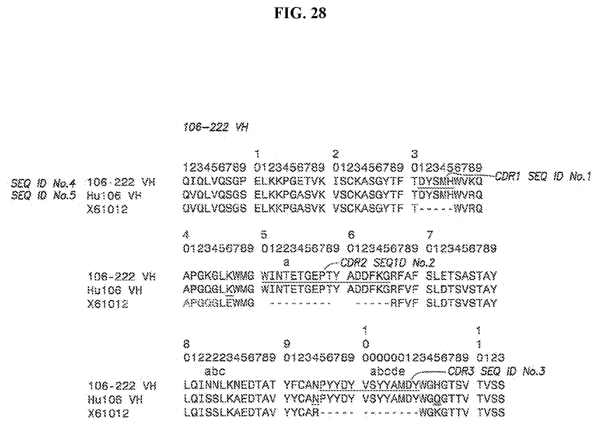

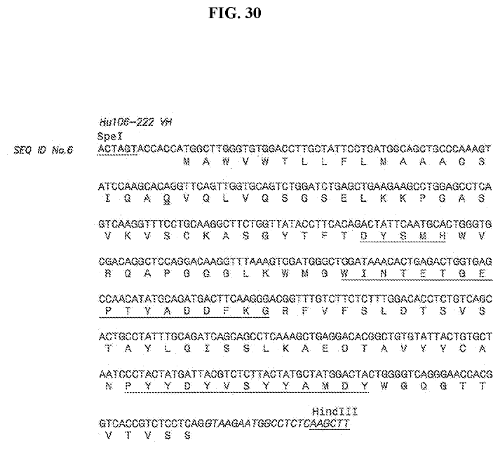

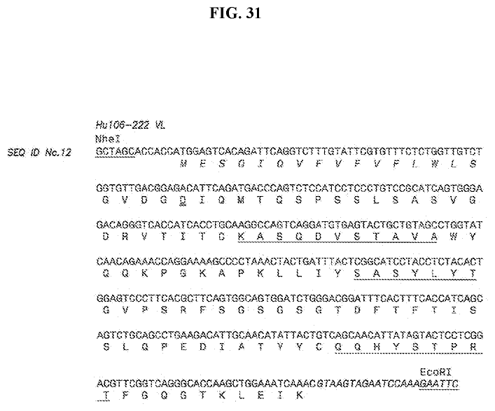

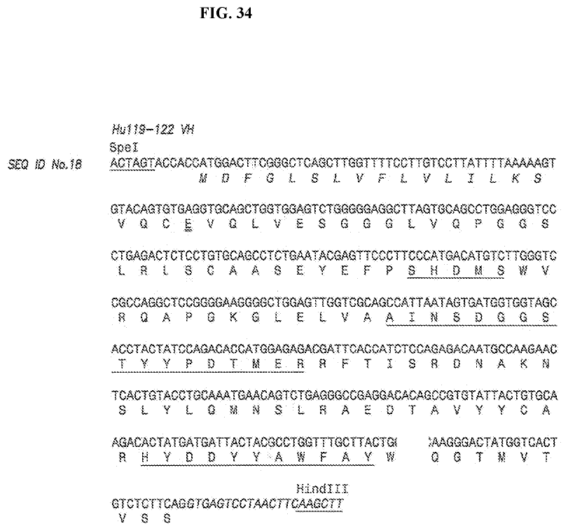

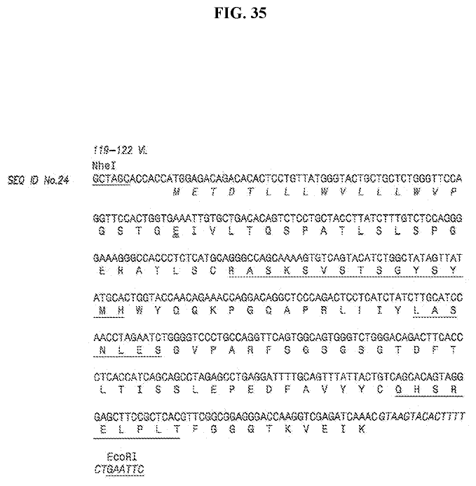

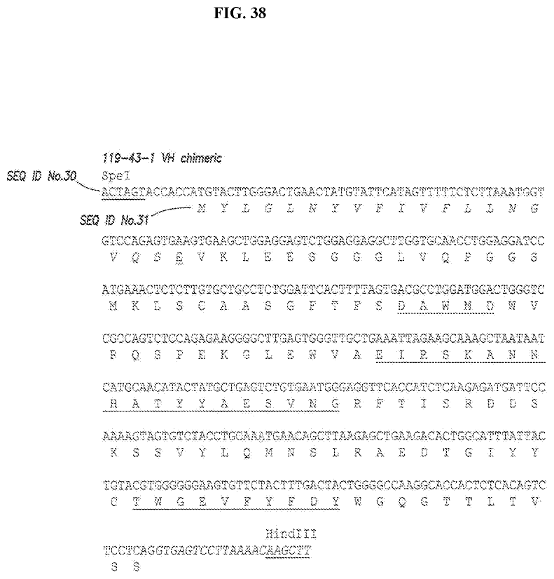

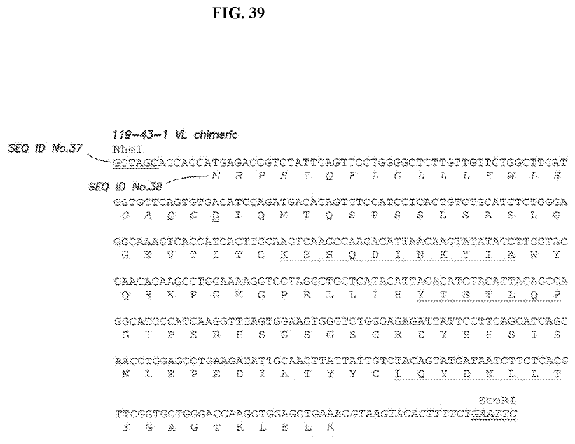

In another embodiment, the anti-OX40 ABP or antibody of the invention comprises one or more of the CDRs or VH or VL sequences, or sequences with 90% identity thereto, shown in FIGS. 28 to 39 herein.

In one embodiment, the anti-OX40 ABP or antibody of the present invention comprise any one or a combination of the following CDRs:

CDRH1:
(SEQ ID NO: 1)
DYSMH
CDRH2:
(SEQ ID NO: 2)
WINTETGEPTYADDFKG
CDRH3:
(SEQ ID NO: 3)
PYYDYVSYYAMDY
CDRL1:
(SEQ ID NO: 7)
KASQDVSTAVA
CDRL2:
(SEQ ID NO: 8)
SASYLYT
CDRL3:
(SEQ ID NO: 9)
QQHYSTPRT

In some embodiments, the anti-OX40 ABP or antibodies of the present invention comprise a heavy chain variable region having at least 90% sequence identity to SEQ ID NO:5. Suitably, the OX40 binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:5.

### Humanized Heavy Chain (V_{H}) Variable Region:

(SEQ ID NO: 5)
QVQLVQSGS ELKKPGASVK VSCKASGYTF TDYSMHWVRQ
APGQGLKWMG WINTETGEPTY ADDFKGRFVF SLDTSVSTAY
LQISSLKAEDTAV YYCANPYYDY VSYYAMDYWGGGTTV TVSS

In one embodiment of the present invention the OX40 ABP or antibody comprises CDRL1 (SEQ ID NO:7), CDRL2 (SEQ ID NO:8), and CDRL3 (SEQ ID NO:9) in the light chain variable region having the amino acid sequence set forth in SEQ ID NO:11. In some embodiments, OX40 binding proteins of the present invention comprise the light chain variable region set forth in SEQ ID NO:11. In one embodiment, an OX40 binding protein of the present invention comprises the heavy chain variable region of SEQ ID NO:5 and the light chain variable region of SEQ ID NO:11.

### Humanized Light Chain (V_{L}) Variable Region

(SEQ ID NO: 11)
DIQMTQSPS SLSASVGDRV TITCKASQDV STAVAWYQQQK
PGKAPKLLIY SASYLYTGVP SRFSGSGSGT DFTFTISSLQ
PEDIATYYCQ QHYSTPRTFG QGTKLEIK

In some embodiments, the OX40 binding proteins of the present invention comprise a light chain variable region having at least 90% sequence identity to the amino acid sequence set forth in SEQ ID NO:11. Suitably, the OX40 binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:11.

In another embodiment, the anti-OX40 ABP or antibody of the present invention comprise any one or a combination of the following CDRs:

CDRH1:
(SEQ ID NO: 13)
SHDMS
CDRH2:
(SEQ ID NO: 14)
AINSDGGSTYYPDTMER
CDRH3:
(SEQ ID NO: 15)
HYDDYYAWFAY
CDRL1:
(SEQ ID NO: 19)
RASKSVSTSGYSYMH
CDRL2:
(SEQ ID NO: 20)
LASNLES
CDRL3:
(SEQ ID NO: 21)
QHSRELPLT

In some embodiments, the anti-OX40 ABP or antibodies of the present invention comprise a heavy chain variable region having at least 90% sequence identity to SEQ ID NO:17. Suitably, the OX40 binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:17.

### Humanized Heavy Chain (V_{H}) Variable Region:

(SEQ ID NO: 17)
EVQLVESGG GLVQPGGSLR LSCAASEYEF PSHDMSWVRQ
APGKGLELVA AINSDGGSTYY PDTMERRFTI SRDNAKNSLY
LQMNSLRAEDTAV YYCARHYDDY YAWFAYWGQGTMV TVSS

In one embodiment of the present invention the OX40 ABP or antibody comprises CDRL1 (SEQ ID NO:19), CDRL2 (SEQ ID NO:20), and CDRL3 (SEQ ID NO:21) in the light chain variable region having the amino acid sequence set forth in SEQ ID NO:23. In some embodiments. OX40 binding proteins of the present invention comprise the light chain variable region set forth in SEQ ID NO:23. In one embodiment, an OX40 binding protein of the present invention comprises the heavy chain variable region of SEQ ID NO:17 and the light chain variable region of SEQ ID NO:23.

### Humanized Light Chain (V_{L}) Variable Region

(SEQ ID NO: 23)
EIVLTQSPA TLSLSPGERA TLSCRASKSVSTSG YSYMHWYQQK
PGQAPRLLIY LASNLESGVP ARFSGSGSGT DFTLTISSLE
PEDFAVYYCQ HSRELPLTFG GGTKVEIK

In some embodiments, the OX40 binding proteins of the present invention comprise a light chain variable region having at least 90% sequence identity to the amino acid sequence set forth in SEQ ID NO:23. Suitably, the OX40 binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:23.

CDRs or minimum binding units may be modified by at least one amino acid substitution, deletion or addition, wherein the variant antigen binding protein substantially retains the biological characteristics of the unmodified protein, such as an antibody comprising SEQ ID NO:5 and SEQ ID NO:11 or an antibody comprising SEQ ID NO: 17 and SEQ ID NO: 23.

It will be appreciated that each of CDR H1, H2, H3, L1, L2, L3 may be modified alone or in combination with any other CDR in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Error! Reference source not found. below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

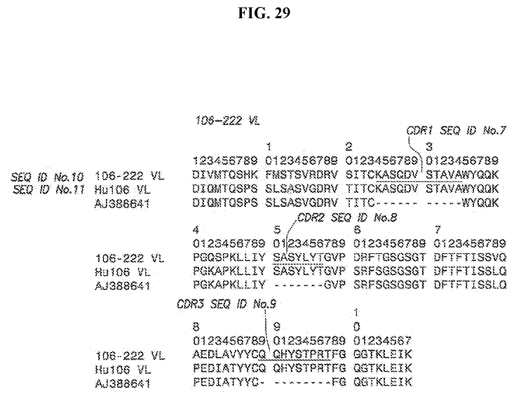

In one embodiment, the ABP or antibody of the invention comprises the CDRs of the 106-222 antibody, e.g., of FIGS. 28-29 herein, e.g., CDRH1, CDRH2, and CDRH3 having the amino acid sequence as set forth in SEQ ID NOs 1, 2, and 3, as disclosed in FIG. 28, and e.g., CDRL1, CDRL2, and CDRL3 having the sequences as set forth in SEQ ID NOs 7, 8, and 9 respectively. In one embodiment, the ABP or antibody of the invention comprises the CDRs of the 106-222, Hu106 or Hu106-222 antibody as disclosed in WO2012/027328 (PCT/US2011/048752), international filing date 23 Aug. 2011. In a further embodiment, the anti-OX40 ABP or antibody of the invention comprises the VH and VL regions of the 106-222 antibody as shown in FIGS. 28-29 herein, e.g., a VH having an amino acid sequence as set forth in SEQ ID NO:4 and a VL as in FIG. 29 having an amino acid sequence as set forth in SEQ ID NO: 10. In another embodiment, the ABP or antibody of the invention comprises a VH having an amino acid sequence as set forth in SEQ ID NO: 5 in FIG. 28 herein, and a VL having an amino acid sequence as set forth in SEQ ID NO:11 in FIG. 29 herein. In a further embodiment, the anti-OX40 ABP or antibody of the invention comprises the VH and VL regions of the Hu106-222 antibody or the 106-222 antibody or the Hu106 antibody as disclosed in WO2012/027328 (PCT/US2011/048752), international filing date 23 Aug. 2011. In a further embodiment, the anti-OX40 ABP or antibody of the invention is 106-222, Hu106-222 or Hu106, e.g., as disclosed in WO2012/027328 (PCT/US2011/048752), international filing date 23 Aug. 2011. In a further embodiment, the ABP or antibody of the invention comprises CDRs or VH or VL or antibody sequences with 90% identity to the sequences in this paragraph.

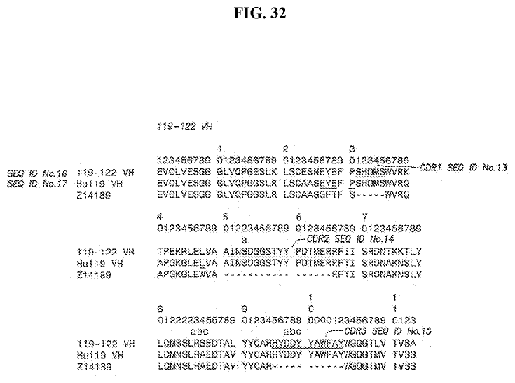

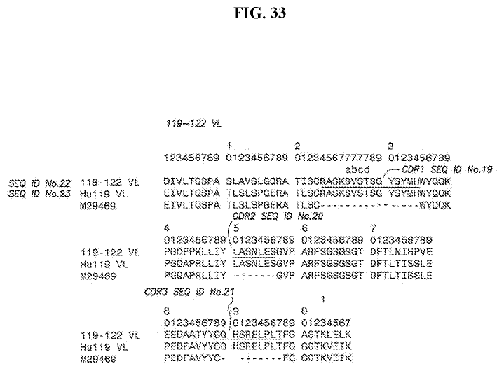

In another embodiment, the anti-OX40 ABP or antibody of the invention comprises the CDRs of the 119-122 antibody, e.g., of FIGS. 32-33 herein, e.g., CDRH1, CDRH2, and CDRH3 having the amino acid sequence as set forth in SEQ ID NOs 13, 14, and 15 respectively. In another embodiment, the anti-OX40 ABP or antibody of the invention comprises the CDRs of the 119-122 or Hut 19 or Hut 19-222 antibody as disclosed in WO2012/027328 (PCT/US2011/048752), international filing date 23 Aug. 2011. In a further embodiment, the anti-OX40 ABP or antibody of the invention comprises a VH having an amino acid sequence as set forth in SEQ ID NO: 16 in FIG. 32 herein, and a VL having the amino acid sequence as set forth in SEQ ID NO: 22 as shown in FIG. 33 herein. In another embodiment, the anti-OX40 ABP or antibody of the invention comprises a VH having an amino acid sequence as set forth in SEQ ID NO: 17 and a VL having the amino acid sequence as set forth in SEQ ID NO: 23. In a further embodiment, the anti-OX40 ABP or antibody of the invention comprises the VH and VL regions of the 119-122 or Hu119 or Hu119-222 antibody as disclosed in WO2012/027328 (PCT/US2011/048752), international filing date 23 Aug. 2011. In a further embodiment, the ABP or antibody of the invention is 119-222 or Hut 19 or Hu119-222 antibody, e.g., as disclosed in WO2012/027328 (PCT/US2011/048752), international filing date 23 Aug. 2011. In a further embodiment, the ABP or antibody of the invention comprises CDRs or VH or VL or antibody sequences with 90% identity to the sequences in this paragraph.

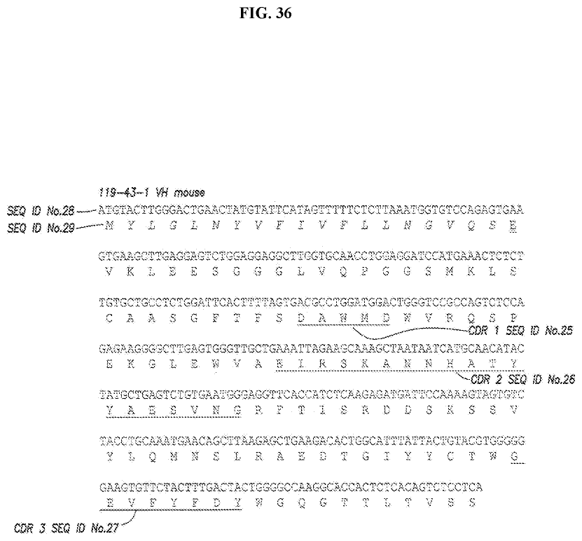

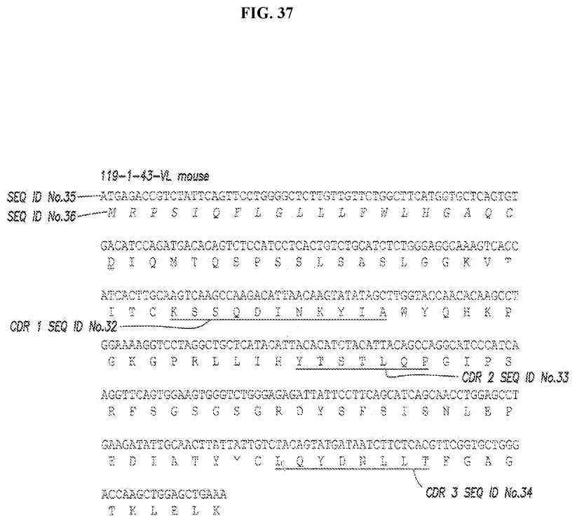

In another embodiment, the anti-OX40 ABP or antibody of the invention comprises the CDRs of the 119-43-1 antibody, e.g., as shown in FIGS. 36-37 herein. In another embodiment, the anti-OX40 ABP or antibody of the invention comprises the CDRs of the 119-43-1 antibody as disclosed in WO2013.%028231 (PCT/US2012/024570), international filing date 9 Feb. 2012. In a further embodiment, the anti-OX40 ABP or antibody of the invention comprises one of the VH and one of the VL regions of the 119-43-1 antibody as shown in FIGS. 36-39. In a further embodiment, the anti-OX40 ABP or antibody of the invention comprises the VH and VL regions of the 119-43-1 antibody as disclosed in WO2013/028231 (PCT/US2012/024570), international filing date 9 Feb. 2012. In a further embodiment, the ABP or antibody of the invention is 119-43-1 or 119-43-1 chimeric as disclosed in FIGS. 36-39 herein. In a further embodiment, the ABP or antibody of the invention as disclosed in WO2013/028231 (PCT/US2012/024570), international filing date 9 Feb. 2012. In further embodiments, any one of the ABPs or antibodies described in this paragraph are humanized. In further embodiments, any one of the any one of the ABPs or antibodies described in this paragraph are engineered to make a humanized antibody. In a further embodiment, the ABP or antibody of the invention comprises CDRs or VH or VL or antibody sequences with 90% identity to the sequences in this paragraph.

In another embodiment, any mouse or chimeric sequences of any anti-OX40 ABP or antibody of the invention are engineered to make a humanized antibody.

In one embodiment, the anti-OX40 ABP or antibody of the invention comprises: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (1) a light chain variable region CDR3 comprising the amino acid sequence of SEQ ID NO. 9.

In another embodiment, the anti-OX40 ABP or antibody of the invention comprises: (a) a heavy chain variable region CDR1 comprising the amino acid sequence of SEQ ID NO: 13; (b) a heavy chain variable region CDR2 comprising the amino acid sequence of SEQ ID NO: 14; (c) a heavy chain variable region CDR3 comprising the amino acid sequence of SEQ ID NO. 15; (d) a light chain variable region CDR1 comprising the amino acid sequence of SEQ ID NO. 19: (e) a light chain variable region CDR2 comprising the amino acid sequence of SEQ ID NO. 20; and (I) a light chain variable region CDR3 comprising the amino acid sequence of SEQ ID NO. 21.

In another embodiment, the anti-OX40 ABP or antibody of the invention comprises: a heavy chain variable region CDR1 comprising the amino acid sequence of SEQ ID NO: 1 or 13; a heavy chain variable region CDR2 comprising the amino acid sequence of SEQ ID NO: 2 or 14; and/or a heavy chain variable region CDR3 comprising the amino acid sequence of SEQ ID NO: 3 or 15, or a heavy chain variable region CDR having 90% identity thereto.

In yet another embodiment, the anti-OX40 ABP or antibody of the invention comprises: a light chain variable region CDR1 comprising the amino acid sequence of SEQ ID NO: 7 or 19; a light chain variable region CDR2 comprising the amino acid sequence of SEQ ID NO: 8 or 20 and/or a light chain variable region CDR3 comprising the amino acid sequence of SEQ ID NO: 9 or 21, or a heavy chain variable region having 90 percent identity thereto.

In a further embodiment, the anti-OX40 ABP or antibody of the invention comprises: a light chain variable region (“VL”) comprising the amino acid sequence of SEQ ID NO: 10, 11, 22 or 23, or an amino acid sequence with at least 90 percent identity to the amino acid sequences of SEQ ID NO: 10, 11, 22 or 23. In another embodiment, the anti-OX40 ABP or antibody of the invention comprises a heavy chain variable region (“VH”) comprising the amino acid sequence of SEQ ID NO: 4, 5, 16 and 17, or an amino acid sequence with at least 90 percent identity to the amino acid sequences of SEQ ID NO: 4, 5, 16 and 17. In another embodiment, the anti-OX40 ABP or antibody of the invention comprises a variable heavy chain sequence of SEQ ID NO:5 and a variable light chain sequence of SEQ ID NO: 11, or a sequence having 90 percent identity thereto. In another embodiment, the anti-OX40 ABP or antibody of the invention comprises a variable heave chain sequence of SEQ ID NO:17 and a variable light chain sequence of SEQ ID NO: 23 or a sequence having 90 percent identity thereto.

In another embodiment, the anti-OX40 ABP or antibody of the invention comprises a variable light chain encoded by the nucleic acid sequence of SEQ ID NO: 12, or 24, or a nucleic acid sequence with at least 90 percent identity to the nucleotide sequences of SEQ ID NO: 12 or 24. In another embodiment, the anti-OX40 ABP or antibody of the invention comprises a variable heavy chain encoded by a nucleic acid sequence of SEQ ID NO: 6 or 18, or a nucleic acid sequence with at least 90 percent identity to nucleotide sequences of SEQ ID NO: 6 or 18.

Also provided herein are monoclonal antibodies. In one embodiment, the monoclonal antibodies comprise a variable light chain comprising the amino acid sequence of SEQ ID NO: 10 or 22, or an amino acid sequence with at least 90 percent identity to the amino acid sequences of SEQ ID NO: 10 or 22. Further provided are monoclonal antibodies comprising a variable heavy chain comprising the amino acid sequence of SEQ ID NO: 4 or 16, or an amino acid sequence with at least 90 percent identity to the amino acid sequences of SEQ ID NO: 4 or 16.

CTLA-4 is a T cell surface molecule that was originally identified by differential screening of a murine cytolytic T cell cDNA library (Brunet et al., Nature 328:267-270(1987)). CTLA-4 is also a member of the immunoglobulin (Ig) superfamily; CTLA-4 comprises a single extracellular Ig domain. CTLA-4 transcripts have been found in T cell populations having cytotoxic activity, suggesting that CTLA-4 might function in the cytolytic response (Brunet et al., supra; Brunet et al., Immunol. Rev. 103-(21-36 (1988)). Researchers have reported the cloning and mapping of a gene for the human counterpart of CTLA-4 (Dariavach et al., Eur. J. Immunol. 18:1901-1905 (1988)) to the same chromosomal region (2q33-34) as CD28 (Lafage-Pochitaloff et al., Immunogenetics 31:198-201 (1990)). Sequence comparison between this human CTLA-4 DNA and that encoding CD28 proteins reveals significant homology of sequence, with the greatest degree of homology in the juxtamembrane and cytoplasmic regions (Brunet et al., 1988, supra; Dariavach et al., 1988, supra). Yervoy (ipilimumab) is a fully human CTLA-4 antibody marketed by Bristol Myers Squibb. The protein structure of ipilimumab and methods are using are described in U.S. Pat. Nos. 6,984,720 and 7,605,238.

Suitable anti-CTLA4 antibodies for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, ipilimumab, tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Published Application No. US 2005/0201994, and the antibodies disclosed in granted European Patent No. EP1212422B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. US 2002/0039581 and US 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145):Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

As used herein an “immuno-modulator” or “immuno-modulatory agent” refers to any substance including monoclonal antibodies that affects the immune system. In some embodiments, the immuno-modulator or immuno-modulatory agent upregulates the immune system. Immuno-modulators can be used as anti-neoplastic agents for the treatment of cancer. For example, immune-modulators include, but are not limited to, anti-PD-1 antibodies (Opdivo/nivolumab and Keytruda/pembrolizumab), anti-CTLA-4 antibodies such as ipilimumab (YERVOY), and anti-OX40 antibodies.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, decrease, abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production.

As used herein the term “cross competes for binding” refers to any agent such as an antibody that will compete for binding to a target with any of the agents of the present invention. Competition for binding between two antibodies can be tested by various methods known in the art including Flow cytometry, Meso Scale Discovery; and ELISA. Binding can be measured directly, meaning two or more binding proteins can be put in contact with a co-signalling receptor and bind may be measured for one or each. Alternatively, binding of molecules or interest can be tested against the binding or natural ligand and quantitatively compared with each other.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., V_{H}, V_{HH}, V_{L}, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing (for a summary of alternative “antibody” formats see, e.g., Holliger and Hudson, Nature Biotechnology, 2005, Vol 23, No. 9, 1126-1136).

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as V_{H}, V_{HH} and V_{L} and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb™” may be considered the same as a “single variable domain”. A single variable domain may be a human single variable domain, but also includes single variable domains from other species such as rodent nurse shark and Camelid V_{HH} dAbs™. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “single variable domains”. As used herein V_{H} includes camelid V_{HH} domains.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

Affinity is the strength of binding of one molecule, e.g. an antigen binding protein of the invention, to another, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE™ analysis). For example, the Biacore™ methods described in Example 5 may be used to measure binding affinity.

Avidity is the sum total of the strength of binding of two molecules to one another at multiple sites, e.g. taking into account the valency of the interaction.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. Such expression vectors may be, for example, cosmids, plasmids, viral sequences, transposons, and linear nucleic acids comprising a nucleic acid of interest. Once the expression vector is introduced into a cell or cell free expression system (e.g., reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g., pcDNA3.1, pCEP4, and their derivatives. Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g., pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof. Most preferably, the eukaryotic cell is a HEK293, NS0, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromasomal or stably integrated into the chromosome of the cell.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson, et al., Bio/Technology, 9:421 (1991)). A suitable human acceptor antibody may be one selected from a conventional database, e.g., the KABAT™ database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

In one embodiment, a combination of a Type I protein arginine methyltransferase (Type I PRMT) inhibitor and an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof, is provided. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In another aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, a combination of a Type I protein arginine methyltransferase (Type I PRMT) inhibitor and an immuno-modulatory agent is provided, wherein the Type I PRMT inhibitor is Compound A and the immuno-modulatory agent is an agonist anti-OX40 antibody or antigen binding fragment thereof. In one embodiment, a combination of a Type I protein arginine methyltransferase (Type I PRMT) inhibitor and an immuno-modulatory agent is provided, wherein the Type I PRMT inhibitor is Compound A and the immuno-modulatory agent is an antagonistic anti-PD1-antibody or antigen binding fragment thereof. In one embodiment, a combination of a Type I protein arginine methyltransferase (Type I PRMT) inhibitor and an immuno-modulatory agent is provided, wherein the Type I PRMT inhibitor is Compound A and the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In one embodiment, a combination of a Type I protein arginine methyltransferase (Type I PRMT) inhibitor and an immuno-modulatory agent is provided, wherein the Type I PRMT inhibitor is Compound A and the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one embodiment, a combination of a Type I protein arginine methyltransferase (Type I PRMT) inhibitor and an immuno-modulatory agent is provided, wherein the Type I PRMT inhibitor is Compound A and the immuno-modulatory agent is an anti-PD1-antibody or antigen binding fragment thereof, wherein the anti-PD1-antibody is pembrolizumab or nivolumab.

In another embodiment, the present invention provides a pharmaceutical composition comprising a therapeutically effective amount of a Type I protein arginine methyltransferase (Type I PRMT) inhibitor and a second pharmaceutical composition comprising a therapeutically effective amount of an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In one aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In another aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, the present invention provides a pharmaceutical composition comprising a therapeutically effective amount of a Type I protein arginine methyltransferase (Type I PRMT) inhibitor and a second pharmaceutical composition comprising a therapeutically effective amount of an immuno-modulatory agent, wherein the Type I PRMT inhibitor is Compound A and the immuno-modulatory agent is an agonist anti-OX40 antibody or antigen binding fragment thereof. In another embodiment, a pharmaceutical composition comprising a therapeutically effective amount of a Type I protein arginine methyltransferase (Type I PRMT) inhibitor and a second pharmaceutical composition comprising a therapeutically effective amount of an immuno-modulatory agent are provided, wherein the Type I PRMT inhibitor is Compound A and the immuno-modulatory agent is an antagonistic anti-PD1-antibody. In one embodiment, a pharmaceutical composition comprising a therapeutically effective amount of a Type I protein arginine methyltransferase (Type I PRMT) inhibitor and a second pharmaceutical composition comprising a therapeutically effective amount of an immuno-modulatory agent are provided, wherein the Type I PRMT inhibitor is Compound A and the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In another embodiment, a pharmaceutical composition comprising a therapeutically effective amount of a Type I protein arginine methyltransferase (Type I PRMT) inhibitor and a second pharmaceutical composition comprising a therapeutically effective amount of an immuno-modulatory agent are provided, wherein the Type I PRMT inhibitor is Compound A and the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one embodiment, a pharmaceutical composition comprising a therapeutically effective amount of a Type I protein arginine methyltransferase (Type I PRMT) inhibitor and a second pharmaceutical composition comprising a therapeutically effective amount of an immuno-modulatory agent are provided, wherein the Type I PRMT inhibitor is Compound A and and the immuno-modulatory agent is an anti-PD1-antibody or antigen binding fragment thereof, wherein the anti-PD1-antibody is pembrolizumab or nivolumab.

In yet another embodiment, methods are provided for treating cancer in a human in need thereof, the methods comprising administering to the human a combination of a Type I protein arginine methyltransferase (Type I PRMT) inhibitor and an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof, is provided., together with at least one of: a pharmaceutically acceptable carrier and a pharmaceutically acceptable diluent, thereby treating the cancer in the human. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In one aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one aspect, the Type I PRMT inhibitor and the immuno-modulatory agent are administered to the patient in a route selected from: simultaneously, sequentially, in any order, systemically, orally, intravenously, and intratumorally. In one aspect, the Type I PRMT inhibitor is administered orally. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, methods are provided for treating cancer in a human in need thereof, the methods comprising administering to the human a combination of Compound A and an agonist anti-OX40 antibody or antigen binding fragment thereof. In another embodiment, methods are provided for treating cancer in a human in need thereof, the methods comprising administering to the human a combination of Compound A and an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In still another embodiment, methods are provided for treating cancer in a human in need thereof, the methods comprising administering to the human a combination of Compound A and an anti-OX40 antibody or antigen binding fragment thereof comprising a heavy chain variable region having at least 90% sequence identity to SEQ ID NO:5 and a light chain variable region having at least 90% identity to SEQ ID NO: 11. In another embodiment, methods are provided for treating cancer in a human in need thereof, the methods comprising administering to the human a combination of Compound A and an antagonist anti-PD1 antibody or antigen binding fragment thereof. In one embodiment, methods are provided for treating cancer in a human in need thereof, the methods comprising administering to the human a combination of Compound A and an anti-PD1 antibody or antigen binding fragment thereof, wherein the anti-PD1-antibody is pembrolizumab or nivolumab.

In a further embodiment, methods are provided for treating cancer in a human in need thereof, the methods comprising administering to the human a therapeutically effective amount of a pharmaceutical composition comprising a Type I protein arginine methyltransferase (Type I PRMT) inhibitor and a pharmaceutical composition comprising an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof, thereby treating the cancer in the human. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In one aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one aspect, the Type I PRMT inhibitor and the immuno-modulatory agent are administered to the patient in a route selected from: simultaneously, sequentially, in any order, systemically, orally, intravenously, and intratumorally. In one aspect, the Type I PRMT inhibitor is administered orally. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, methods are provided for treating cancer in a human in need thereof, the methods comprising administering to the human a therapeutically effective amount of a pharmaceutical composition comprising Compound A and a pharmaceutical composition comprising an agonist anti-OX40 antibody or antigen binding fragment thereof. In another embodiment, methods are provided for treating cancer in a human in need thereof, the methods comprising administering to the human a therapeutically effective amount of a pharmaceutical composition comprising Compound A and a pharmaceutical composition comprising an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In still another embodiment, methods are provided for treating cancer in a human in need thereof, the methods comprising administering to the human a therapeutically effective amount of a pharmaceutical composition comprising Compound A and a pharmaceutical composition comprising an anti-OX40 antibody or antigen binding fragment thereof comprising a heavy chain variable region having at least 90% sequence identity to SEQ ID NO:5 and a light chain variable region having at least 90% identity to SEQ ID NO: 11. In another embodiment, methods are provided for treating cancer in a human in need thereof, the methods comprising administering to the human a therapeutically effective amount of a pharmaceutical composition comprising Compound A and a pharmaceutical composition comprising an antagonist anti-PD1 antibody or antigen binding fragment thereof. In one embodiment, methods are provided for treating cancer in a human in need thereof, the methods comprising administering to the human a therapeutically effective amount of a pharmaceutical composition comprising of Compound A and a pharmaceutical composition comprising an anti-PD1 antibody or antigen binding fragment thereof, wherein the anti-PD1-antibody is pembrolizumab or nivolumab.

In another embodiment, the present invention provides use of a combination of a Type I protein arginine methyltransferase (Type I PRMT) inhibitor and an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof, for the manufacture of a medicament. In one embodiment, the present invention provides use of a combination of a Type I protein arginine methyltransferase (Type I PRMT) inhibitor and an immuno-modulatory agent selected from: an anti-PD-1 antibody or antigen binding fragment thereof, an anti-PDL1 antibody or antigen binding fragment thereof, and an anti-OX40 antibody or antigen binding fragment thereof, for the treatment of cancer. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In one aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, use of a combination of a Type I protein arginine methyltransferase (Type I PRMT) inhibitor and an immuno-modulatory agent is provided for the manufacture of a medicament, wherein the Type I PRMT inhibitor is Compound A and the immuno-modulatory agent is an agonist anti-OX40 antibody or antigen binding fragment thereof. In one embodiment, use of a combination of a Type I protein arginine methyltransferase (Type I PRMT) inhibitor and an immuno-modulatory agent for the manufacture of a medicament is provided, wherein the Type I PRMT inhibitor is Compound A and the immuno-modulatory agent is an antagonistic anti-PD1-antibody or antigen binding fragment thereof. In one embodiment, use of a combination of a Type I protein arginine methyltransferase (Type I PRMT) inhibitor and an immuno-modulatory agent for the manufacture of a medicament is provided, wherein the Type I PRMT inhibitor is Compound A and the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In one embodiment, use of a combination of a Type I protein arginine methyltransferase (Type I PRMT) inhibitor and an immuno-modulatory agent for the manufacture of a medicament is provided, wherein the Type I PRMT inhibitor is Compound A and the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one embodiment, use of a combination of a Type I protein arginine methyltransferase (Type I PRMT) inhibitor and an immuno-modulatory agent for the manufacture of a medicament is provided, wherein the Type I PRMT inhibitor is Compound A and the immuno-modulatory agent is an anti-PD1-antibody or antigen binding fragment thereof, wherein the anti-PD1-antibody is pembrolizumab or nivolumab.

In one embodiment, the present invention provides a combination of a Type I protein arginine methyltransferase (Type I PRMT) inhibitor and an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof, for use in the treatment of cancer. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In one aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one aspect, the Type I PRMT inhibitor and the immuno-modulatory agent are administered to the patient in a route selected from: simultaneously, sequentially, in any order, systemically, orally, intravenously, and intratumorally. In one aspect, the Type I PRMT inhibitor is administered orally. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, a combination of a Type I protein arginine methyltransferase (Type I PRMT) inhibitor and an immuno-modulatory agent for use in the treatment of cancer is provided, wherein the Type I PRMT inhibitor is Compound A and the immuno-modulatory agent is an agonist anti-OX40 antibody or antigen binding fragment thereof. In one embodiment, a combination of a Type I protein arginine methyltransferase (Type I PRMT) inhibitor and an immuno-modulatory agent for use in the treatment of cancer is provided, wherein the Type I PRMT inhibitor is Compound A and the immuno-modulatory agent is an antagonistic anti-PD1-antibody or antigen binding fragment thereof. In one embodiment, a combination of a Type I protein arginine methyltransferase (Type I PRMT) inhibitor and an immuno-modulatory agent for use in the treatment of cancer is provided, wherein the Type I PRMT inhibitor is Compound A and the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In one embodiment, a combination of a Type I protein arginine methyltransferase (Type I PRMT) inhibitor and an immuno-modulatory agent for use in the treatment of cancer is provided, wherein the Type I PRMT inhibitor is Compound A and the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one embodiment, a combination of a Type I protein arginine methyltransferase (Type I PRMT) inhibitor and an immuno-modulatory agent for use in the treatment of cancer is provided, wherein the Type I PRMT inhibitor is Compound A and the immuno-modulatory agent is an anti-PD1-antibody or antigen binding fragment thereof, wherein the anti-PD1-antibody is pembrolizumab or nivolumab.

In one embodiment, a product containing a Type I PRMT inhibitor and an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof as a combined preparation for simultaneous, separate, or sequential use in medicine is provided. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In one aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one aspect, the Type I PRMT inhibitor and the immuno-modulatory agent are administered to the patient in a route selected from: simultaneously, sequentially, in any order, systemically, orally, intravenously, and intratumorally. In one aspect, the Type I PRMT inhibitor is administered orally. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, a product containing Compound A and an agonist anti-OX40 antibody or antigen binding fragment thereof for simultaneous, separate, or sequential use in medicine is provided. In another embodiment, a product containing Compound A and an antagonist anti-PD1 antibody for simultaneous, separate, or sequential use in medicine is provided. In one embodiment, a product containing Compound A and an anti-OX40 antibody or antigen binding fragment thereof for simultaneous, separate, or sequential use in medicine is provided, wherein the anti-OX40 antibody or antigen binding fragment thereof comprises one or more of: CDRH1 as set forth in SEQ ID NO: 1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In one embodiment, a product containing Compound A and an anti-OX40 antibody or antigen binding fragment thereof for simultaneous, separate, or sequential use in medicine is provided, wherein the anti-OX40 antibody or antigen binding fragment thereof comprises a heavy chain variable region having at least 90% sequence identity to SEQ ID NO:5 and a light chain variable region having at least 90% identity to SEQ ID NO: 11. In another embodiment, a product containing Compound A and an anti-PD1 antibody or antigen binding fragment thereof for simultaneous, separate, or sequential use in medicine is provided, wherein the anti-PD-antibody is pembrolizumab or nivolumab.

In one embodiment, a product containing a Type I PRMT inhibitor and an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof as a combined preparation for simultaneous, separate, or sequential use in treating cancer in a human subject is provided. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In one aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO: 7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one aspect, the Type I PRMT inhibitor and the immuno-modulatory agent are administered to the patient in a route selected from: simultaneously, sequentially, in any order, systemically, orally, intravenously, and intratumorally. In one aspect, the Type I PRMT inhibitor is administered orally. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, a product containing Compound A and an agonist anti-OX40 antibody or antigen binding fragment thereof for simultaneous, separate, or sequential use in treating cancer in a human subject is provided. In another embodiment, a product containing Compound A and an antagonist anti-PD1 antibody or antigen binding fragment thereof for simultaneous, separate, or sequential use in treating cancer in a human subject is provided. In one embodiment, a product containing Compound A and an anti-OX40 antibody or antigen binding fragment thereof for simultaneous, separate, or sequential use in treating cancer in a human subject is provided, wherein the anti-OX40 antibody or antigen binding fragment thereof comprises one or more of: CDRH1 as set forth in SEQ ID NO: 1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In one embodiment, a product containing Compound A and an anti-OX40 antibody or antigen binding fragment thereof for simultaneous, separate, or sequential use in treating cancer in a human subject is provided, wherein the anti-OX40 antibody or antigen binding fragment thereof comprises a heavy chain variable region having at least 90% sequence identity to SEQ ID NO:5 and a light chain variable region having at least 90% identity to SEQ ID NO: 11. In another embodiment, a product containing Compound A and an anti-PD1 antibody or antigen binding fragment thereof for simultaneous, separate, or sequential use in treating cancer in a human subject is provided, wherein the anti-PD1-antibody is pembrolizumab or nivolumab.

In one embodiment, a product containing a Type I PRMT inhibitor and an immuno-modulatory agent selected from: an anti-CTLA4 antibody or antigen binding fragment thereof, an anti-PD-1 antibody or antigen binding fragment thereof, an anti-PDL1 antibody or antigen binding fragment thereof, and an anti-OX40 antibody or antigen binding fragment thereof as a combined preparation for simultaneous, separate, or sequential use in treating cancer in a human subject is provided, wherein the cancer is melanoma, colon cancer, or lymphoma. In one aspect, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor. In one aspect, the immuno-modulatory agent is an anti-PD-1 antibody or antigen binding fragment thereof. In one aspect, the anti-PD-1 antibody is pembrolizumab or nivolumab. In another aspect, the immuno-modulatory agent is an anti-OX40 antibody or antigen binding fragment thereof. In still another aspect, the immuno-modulatory agent is an OX40 agonist. In one aspect, the immuno-modulatory agent is an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In another aspect, the immuno-modulatory agent is an anti-OX40 antibody or antigen binding fragment thereof comprising a heavy chain variable region having at least 90% sequence identity to SEQ ID NO:5 and a light chain variable region having at least 90% identity to SEQ ID NO: 11. In one aspect, the Type I PRMT inhibitor and the immuno-modulatory agent are administered to the patient in a route selected from: simultaneously, sequentially, in any order, systemically, orally, intravenously, and intratumorally. In one aspect, the Type I PRMT inhibitor is administered orally. In one aspect, the Type I PRMT inhibitor is a compound of Formula I, II, V, or VI. In one aspect, the Type I PRMT inhibitor is Compound A. In another aspect, the Type I PRMT inhibitor is Compound D. In one embodiment, a product containing Compound A and an agonist anti-OX40 antibody or antigen binding fragment thereof for simultaneous, separate, or sequential use in treating cancer in a human subject is provided, wherein the cancer is colon cancer or lymphoma. In another embodiment, a product containing Compound A and an antagonist anti-PD1 antibody or antigen binding fragment thereof for simultaneous, separate, or sequential use in treating cancer in a human subject is provided, wherein the cancer is melanoma. In one embodiment, a product containing Compound A and an anti-OX40 antibody or antigen binding fragment thereof for simultaneous, separate, or sequential use in treating cancer in a human subject is provided, wherein the cancer is colon cancer or lymphoma, and wherein the anti-OX40 antibody or antigen binding fragment thereof comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR. In one embodiment, a product containing Compound A and an anti-OX40 antibody or antigen binding fragment thereof for simultaneous, separate, or sequential use in treating cancer in a human subject is provided, wherein the cancer is colon cancer or lymphoma, and wherein the anti-OX40 antibody or antigen binding fragment thereof comprises a heavy chain variable region having at least 90% sequence identity to SEQ ID NO:5 and a light chain variable region having at least 90% identity to SEQ ID NO: 11. In another embodiment, a product containing Compound A and an anti-PD1 antibody or antigen binding fragment thereof for simultaneous, separate, or sequential use in treating cancer in a human subject is provided, wherein the cancer is melanoma, and wherein the anti-PD-antibody is pembrolizumab or nivolumab.

In one aspect of any one of the embodiments herein, the cancer is a solid tumor or a haematological cancer. In one aspect, the cancer is melanoma, lymphoma, or colon cancer.

In one aspect the cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor. Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia. Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

In one aspect, the methods of the present invention further comprise administering at least one neo-plastic agent to said human.

In one aspect the human has a solid tumor. In one aspect the tumor is selected from head and neck cancer, gastric cancer, melanoma, renal cell carcinoma (RCC), esophageal cancer, non-small cell lung carcinoma, prostate cancer, colorectal cancer, ovarian cancer and pancreatic cancer. In another aspect the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkins lymphoma, non-hodgkins lymphoma, lymphoblastic T cell lymphoma. Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent Bcell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL): and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood: thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

As used herein the term “Compound A²” means an immuno-modulatory agent selected from: an anti-PD-1 antibody or antigen binding fragment thereof, an anti-PDL1 antibody or antigen binding fragment thereof, an anti-CTLA4 antibody or antigen binding fragment thereof, or an anti-OX40 antibody or antigen binding fragment thereof. In some embodiments, Compound A² is an anti-PD-1 antibody. Suitably Compound A² may be selected from nivolumab and pembrolizumab. In some embodiments, Compound A² is an agonist antibody directed to OX40 or antigen binding portion thereof comprising a V_{H} domain comprising an amino acid sequence at least 90% identical to the amino acid sequence set forth in SEQ ID NO:5; and a V_{L} domain comprising an amino acid sequence at least 90% identical to the amino acid sequence as set forth in SEQ ID NO:11. In still other embodiments, Compound A² is an agonist antibody direct to OX40 or antigen binding portion thereof comprising an anti-OX40 antibody or antigen binding fragment thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR.

As used herein the term “Compound B²” means a Type I PRMT inhibitor. In some embodiments, Compound B² is a compound of Formula I, II, V, or VI. Suitably Compound B² is Compound A.

Suitably, the combinations of this invention are administered within a “specified period”.

The term “specified period” and grammatical variations thereof, as used herein, means the interval of time between the administration of one of Compound A² and Compound B² and the other of Compound A² and Compound B². Unless otherwise defined, the specified period can include simultaneous administration. Unless otherwise defined, the specified period refers to administration of Compound A² and Compound B² during a single day.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours: suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other in this case, the specified period will be about 3 hours: suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

The term “duration of time” and grammatical variations thereof, as used herein means that both compounds of the invention are administered for an indicated number of consecutive days. Unless otherwise defined, the number of consecutive days does not have to commence with the start of treatment or terminate with the end of treatment, it is only required that the number of consecutive days occur at some point during the course of treatment.

### Regarding “Specified Period” Administration:

Suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and grammatical derivates thereof, as used herein is meant that one of Compound A² and Compound B² is administered once a day for two or more consecutive days and the other of Compound A² and Compound B² is subsequently administered once a day for two or more consecutive days. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B2 is administered. Suitably the drug holiday will be a period of days selected from: 1 day, 2 days, 3 days, 4 days, 5 days. 6 days. 7 days, 8 days, 9 days, 10 days, 11 days, 12 days, 13 days and 14 days.

### Regarding Sequential Administration:

Suitably, one of Compound A² and Compound B² is administered for from 1 to 30 consecutive days, followed by an optional drug holiday, followed by administration of the other of Compound A² and Compound B² for from 1 to 30 consecutive days. Suitably, one of Compound A² and Compound B² is administered for from 1 to 21 consecutive days, followed by an optional drug holiday, followed by administration of the other of Compound A² and Compound B² for from 1 to 21 consecutive days. Suitably, one of Compound A² and Compound B² is administered for from 1 to 14 consecutive days, followed by a drug holiday of from 1 to 14 days, followed by administration of the other of Compound A² and Compound B² for from 1 to 14 consecutive days. Suitably, one of Compound A² and Compound B² is administered for from 1 to 7 consecutive days, followed by a drug holiday of from 1 to 10 days, followed by administration of the other of Compound A² and Compound B² for from 1 to 7 consecutive days.

Suitably, Compound B² will be administered first in the sequence, followed by an optional drug holiday, followed by administration of Compound A2. Suitably, Compound B² is administered for from 3 to 21 consecutive days, followed by an optional drug holiday, followed by administration of Compound A² for from 3 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A2 for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably. Compound B² is administered for 3 consecutive days, followed by a drug holiday of from 3 to 10 days, followed by administration of Compound A² for 3 consecutive days.

It is understood that a “specified period” administration and a “sequential” administration can be followed by repeat dosing or can be followed by an alternate dosing protocol, and a drug holiday may precede the repeat dosing or alternate dosing protocol.

The methods of the present invention may also be employed with other therapeutic methods of cancer treatment.

Compound A² and Compound B² may be administered by any appropriate route. Suitable routes include oral, rectal, nasal, topical (including buccal and sublingual), intratumorally, vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

In one embodiment, one or more components of a combination of the invention are administered intravenously. In one embodiment, one or more components of a combination of the invention are administered orally. In another embodiment, one or more components of a combination of the invention are administered intratumorally. In another embodiment, one or more components of a combination of the invention are administered systemically, e.g., intravenously, and one or more other components of a combination of the invention are administered intratumorally. In any of the embodiments, e.g., in this paragraph, the components of the invention are administered as one or more pharmaceutical compositions.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10^{th} edition (Dec. 5, 2014), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents such as diterpenoids and vinca alkaloids; platinum coordination complexes; alkylating agents such as nitrogen mustards, oxazaphosphorines, alkylsulfonates, nitrosoureas, and triazenes; antibiotic agents such as actinomycins, anthracyclins, and bleomycins; topoisomerase I inhibitors such as camptothecins; topoisomerase II inhibitors such as epipodophyllotoxins; antimetabolites such as purine and pyrimidine analogues and anti-folate compounds; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; and cancer gene therapy agents such as genetically modified T cells.

Examples of a further active ingredient or ingredients for use in combination or co-administered with the present methods or combinations are anti-neoplastic agents. Examples of anti-neoplastic agents include, but are not limited to, chemotherapeutic agents; immuno-modulatory agents; immune-modulators; and immunostimulatory adjuvants.

### EXAMPLES

The following examples illustrate various non-limiting aspects of this invention.

### Example 1

### Arginine Methylation and PRMTs

Arginine methylation is an important post-translational modification on proteins involved in a diverse range of cellular processes such as gene regulation, RNA processing, DNA damage response, and signal transduction. Proteins containing methylated arginines are present in both nuclear and cytosolic fractions suggesting that the enzymes that catalyze the transfer of methyl groups on to arginines are also present throughout these subcellular compartments (reviewed in Yang, Y. & Bedford, M. T. Protein arginine methyltransferases and cancer. Nat Rev Cancer 13, 37-50, doi:10.1038/nrc3409 (2013); Lee, Y. H. & Stallcup, M. R. Minireview: protein arginine methylation of nonhistone proteins in transcriptional regulation. Mol Endocrinol 23, 425-433, doi:10.1210/me.2008-0380 (2009)). In mammalian cells, methylated arginine exists in three major forms: ω-N^{G}-monomethyl-arginine (MMA), ω-N^{G},N^{G}-asymmetric dimethyl arginine (ADMA), or ω-N^{G},N′^{G}-symmetric dimethyl arginine (SDMA). Each methylation state can affect protein-protein interactions in different ways and therefore has the potential to confer distinct functional consequences for the biological activity of the substrate (Yang, Y. & Bedford, M. T. Protein arginine methyltransferases and cancer. Nat Rev Cancer 13, 37-50, doi:10.1038/nrc3409 (2013)).

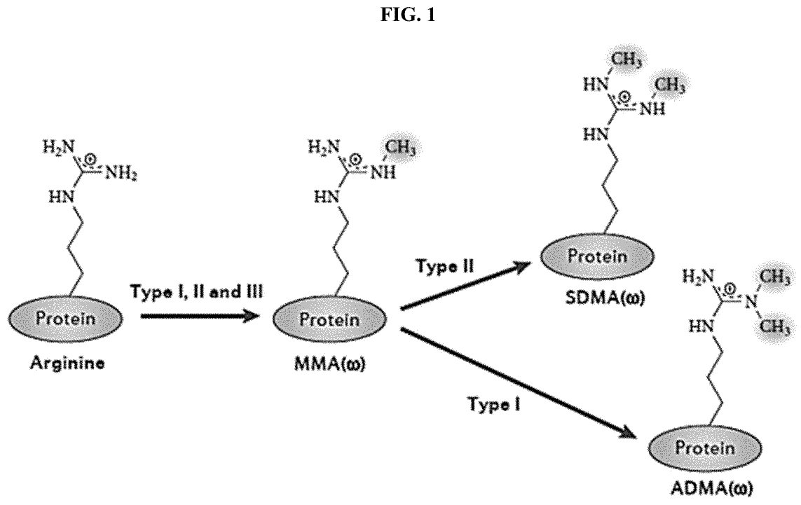

Arginine methylation occurs largely in the context of glycine-, arginine-rich (GAR) motifs through the activity of a family of Protein Arginine Methyltransferases (PRMTs) that transfer the methyl group from S-adenosyl-L-methionine (SAM) to the substrate arginine side chain producing S-adenosyl-homocysteine (SAH) and methylated arginine (FIG. 1). This family of proteins is comprised of 10 members of which 9 have been shown to have enzymatic activity (Bedford, M. T. & Clarke, S. G. Protein arginine methylation in mammals: who, what, and why. Mol Cell 33, 1-13, doi:10.1016/j.molcel.2008.12.013 (2009)). The PRMT family is categorized into four sub-types (Type I-IV) depending on the product of the enzymatic reaction (FIG. 1). Type IV enzymes methylate the internal guanidino nitrogen and have only been described in yeast (Fisk, J. C. & Read, L. K. Protein arginine methylation in parasitic protozoa. Eukaryot Cell 10, 1013-1022, doi: 10.1128/EC.05103-11 (2011)): types I-III enzymes generate monomethyl-arginine (MMA, Rme1) through a single methylation event. The MMA intermediate is considered a relatively low abundance intermediate, however, select substrates of the primarily Type III activity of PRMT7 can remain monomethylated, while Types I and II enzymes catalyze progression from MMA to either asymmetric dimethyl-arginine (ADMA, Rme2a) or symmetric dimethyl arginine (SDMA, Rme2s) respectively. Type II PRMTs include PRMT5, and PRMT9, however, PRMT5 is the primary enzyme responsible for formation of symmetric dimethylation. Type I enzymes include PRMT1, PRMT3, PRMT4, PRMT6 and PRMT8. PRMT1, PRMT3, PRMT4, and PRMT6 are ubiquitously expressed while PRMT8 is largely restricted to the brain (reviewed in Bedford, M. T. & Clarke, S. G. Protein arginine methylation in mammals: who, what, and why. Mol Cell 33, 1-13, doi: 10.1016/j.molcel.2008.12.013 (2009)).

PRMT1 is the primary Type I enzyme capable of catalyzing the formation of MMA and ADMA on numerous cellular substrates (Bedford, M. T. & Clarke, S. G. Protein arginine methylation in mammals: who, what, and why. Mol Cell 33, 1-13, doi: 10.1016/j.molcel.2008.12.013 (2009)). In many instances, the PRMT1-dependent ADMA modification is required for the biological activity and trafficking of its substrates (Nicholson, T. B., Chen, T. & Richard, S. The physiological and pathophysiological role of PRMT1-mediated protein arginine methylation. Pharmacol Res 60, 466-474, doi:10.1016/j.phrs.2009.07.006 (2009)), and the activity of PRMT1 accounts for ˜85% of cellular ADMA levels (Dhar, S. et al. Loss of the major Type I arginine methyltransferase PRMT1 causes substrate scavenging by other PRMTs. Sci Rep 3, 1311, doi:10.1038/srep01311 (2013); Pawlak, M. R., Scherer, C. A., Chen, J., Roshon, M. J. & Ruley, H. E. Arginine N-methyltransferase 1 is required for early postimplantation mouse development, but cells deficient in the enzyme are viable. Mol Cell Biol 20, 4859-4869 (2000)). Complete knockout of PRMT1 results in a profound increase in MMA across numerous substrates suggesting that the major biological function for PRMT1 is to convert MMA to ADMA while other PRMTs can establish and maintain MMA (Dhar, S. et al. Loss of the major Type I arginine methyltransferase PRMT1 causes substrate scavenging by other PRMTs. Sci Rep 3, 1311, doi:10.1038/srep01311 (2013)). In addition, SDMA levels are increased upon loss of PRMT1, likely a consequence of the loss of ADMA and the corresponding increase of MMA that can serve as the substrate for SDMA-generating Type II PRMTs. Inhibition of Type I PRMTs may lead to altered substrate function through loss of ADMA, increase in MMA, or, alternatively, a switch to the distinct methylation pattern associated with SDMA (Dhar, S. et al. Loss of the major Type I arginine methyltransferase PRMT1 causes substrate scavenging by other PRMTs. Sci Rep 3, 1311, doi: 10.1038/srep01311 (2013)).

Disruption of the Prmt1 locus in mice results in early embryonic lethality and homozygous embryos fail to develop beyond E6.5 indicating a requirement for PRMT1 in normal development (Pawlak, M. R., Scherer, C. A., Chen, J., Roshon, M. J. & Ruley, H. E. Arginine N-methyltransferase 1 is required for early postimplantation mouse development, but cells deficient in the enzyme are viable. Mol Cell Biol 20, 4859-4869 (2000); Yu, Z., Chen, T., Hebert, J., Li, E. & Richard, S. A mouse PRMT1 null allele defines an essential role for arginine methylation in genome maintenance and cell proliferation. Mol Cell Biol 29, 2982-29%, doi:10.1128/MCB.00042-09 (2009)). Conditional or tissue specific knockout will be required to better understand the role for PRMT1 in the adult. Mouse embryonic fibroblasts derived from Prmt1 null mice undergo growth arrest, polyploidy, chromosomal instability, and spontaneous DNA damage in association with hypomethylation of the DNA damage response protein MRE11, suggesting a role for PRMT1 in genome maintenance and cell proliferation (Yu, Z., Chen, T., Hebert, J., Li, E. & Richard, S. A mouse PRMT1 null allele defines an essential role for arginine methylation in genome maintenance and cell proliferation. Mol Cell Biol 29, 2982-2996, doi:10.1128/MCB.00042-09 (2009)). PRMT1 protein and mRNA can be detected in a wide range of embryonic and adult tissues, consistent with its function as the enzyme responsible for the majority of cellular arginine methylation. Although PRMTs can undergo post-translational modifications themselves and are associated with interacting regulatory proteins. PRMT1 retains basal activity without a requirement for additional modification (reviewed in Yang, Y. & Bedford. M. T. Protein arginine methyltransferases and cancer. Nat Rev Cancer 13, 37-50, doi:10.1038/nrc3409 (2013)).

### PRMT1 and Cancer

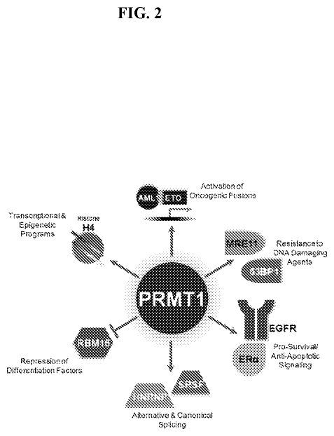

Mis-regulation and overexpression of PRMT1 has been associated with a number of solid and hematopoietic cancers (Yang, Y. & Bedford, M. T. Protein arginine methyltransferases and cancer. Nat Rev Cancer 13, 37-50, doi:10.1038/nrc3409 (2013); Yoshimatsu, M. et al. Dysregulation of PRMT1 and PRMT6, Type I arginine methyltransferases, is involved in various types of human cancers. Int J Cancer 128, 562-573, doi:10.1002/ijc.25366 (2011)). The link between PRMT1 and cancer biology has largely been through regulation of methylation of arginine residues found on relevant substrates (FIG. 2). In several tumor types, PRMT1 can drive expression of aberrant oncogenic programs through methylation of histone H4 (Takai, H. et al. 5-Hydroxymethylcytosine plays a critical role in glioblastomagenesis by recruiting the CHTOP-methylosome complex. Cell Rep 9, 48-60, doi:10.1016/j.celrep.2014.08.071 (2014); Shia, W. J. et al. PRMT1 interacts with AML1-ETO to promote its transcriptional activation and progenitor cell proliferative potential. Blood 119, 4953-4962, doi:10.1182/blood-2011-04-347476 (2012); Zhao, X. et al. Methylation of RUNX1 by PRMT1 abrogates SIN3A binding and potentiates its transcriptional activity. Genes Bev 22, 640-653, doi:10.1101/gad.1632608 (2008)), as well as through its activity on non-histone substrates (Wei, H., Mundade, R., Lange, K. C. & Lu, T. Protein arginine methylation of non-histone proteins and its role in diseases. Cell Cycle 13, 32-41, doi:10.4161/cc.27353 (2014)). In many of these experimental systems, disruption of the PRMT1-dependent ADMA modification of its substrates decreases the proliferative capacity of cancer cells (Yang, Y. & Bedford, M. T. Protein arginine methyltransferases and cancer. Nat Rev Cancer 13, 37-50, doi:10.1038/nrc3409 (2013)).

Several studies have linked PRMT1 to the development of hematological and solid tumors. PRMT1 is associated with leukemia development through methylation of key drivers such as MLL and AML1-ETO fusions, leading to activation of oncogenic pathways (Shia, W. J. et al. PRMT1 interacts with AML1-ETO to promote its transcriptional activation and progenitor cell proliferative potential. Blood 119, 4953-4962, doi:10.1182/blood-2011-04-347476 (2012); Cheung, N. et al. Targeting Aberrant Epigenetic Networks Mediated by PRMT1 and KDM4C in Acute Myeloid Leukemia. Cancer Cell 29, 32-48, doi:10.1016/j.ccell.2015.12.007 (2016)). Knockdown of PRMT1 in bone marrow cells derived from AML1-ETO expressing mice suppressed clonogenicity, demonstrating a critical requirement for PRMT1 in maintaining the leukemic phenotype of this model (Shia, W. J. et al. PRMT1 interacts with AML 1-ETO to promote its transcriptional activation and progenitor cell proliferative potential. Blood 119, 4953-4962, doi:10.1182/blood-2011-04-347476 (2012)). PRMT1 is also a component of MLL fusion complexes, promotes aberrant transcriptional activation in association with H4R3 methylation, and knockdown of PRMT1 can suppress MLL-EEN mediated transformation of hematopoietic stem cells (Cheung, N., Chan, L. C., Thompson, A., Cleary, M. L. & So, C. W. Protein arginine-methyltransferase-dependent oncogenesis. Nat Cell Biol 9, 1208-1215, doi:10.1038/ncb1642 (2007)). In breast cancer patients, high expression of PRMT1 was found to correlate with shorter disease free survival and with tumors of advanced histological grade (Mathioudaki, K. et al. Clinical evaluation of PRMT1 gene expression in breast cancer. Tumour Biol 32, 575-582, doi:10.1007/s 13277-010-0153-2 (2011)). To this end, PRMT1 has been implicated in the promotion of metastasis and cancer cell invasion (Gao. Y. et al. The dual function of PRMT1 in modulating epithelial-mesenchymal transition and cellular senescence in breast cancer cells through regulation of ZEB 1. Sci Rep 6, 19874, doi:10.1038/srep19874 (2016); Avasarala, S. et al. PRMT1 Is a Novel Regulator of Epithelial-Mesenchymal-Transition in Non-small Cell Lung Cancer. J Biol Chem 290, 13479-13489, doi:10.1074/jbc.M114.636050 (2015)) and PRMT1 mediated methylation of Estrogen Receptor α (ERα) can potentiate growth-promoting signal transduction pathways. This methylation driven mechanism may provide a growth advantage to breast cancer cells even in the presence of anti-estrogens (Le Romancer, M. et al. Regulation of estrogen rapid signaling through arginine methylation by PRMT1. Mol Cell 31, 212-221, doi: 10.1016/j.molcel.2008.05.025 (2008)). In addition, PRMT1 promotes genome stability and resistance to DNA damaging agents through regulating both homologous recombination and non-homologous end joining DNA repair pathways (Boisvert, F. M., Rhie, A., Richard, S. & Doherty, A. J. The GAR motif of 53BP1 is arginine methylated by PRMT1 and is necessary for 53BP1 DNA binding activity. Cell Cycle 4, 1834-1841, doi:10.4161/cc.4.12.2250 (2005); Boisvert, F. M., Dery, U., Masson, J. Y. & Richard, S. Arginine methylation of MRE11 by PRMT1 is required for DNA damage checkpoint control. Genes Dev 19, 671-676, doi:10.1101/gad.1279805 (2005)). Therefore, inhibition of PRMT1 may sensitize cancers to DNA damaging agents, particularly in tumors where DNA repair machinery may be compromised by mutations (such as BRCA1 in breast cancers) (O'Donovan, P. J. & Livingston, D. M. BRCA1 and BRCA2: breast/ovarian cancer susceptibility gene products and participants in DNA double-strand break repair. Carcinogenesis 31, 961-967, doi:10.1093/carcin/bgq069 (2010)). Together, these observations demonstrate key roles for PRMT1 in clinically-relevant aspects of tumor biology, and suggest a rationale for exploring combinations with therapies such as those that promote DNA damage.

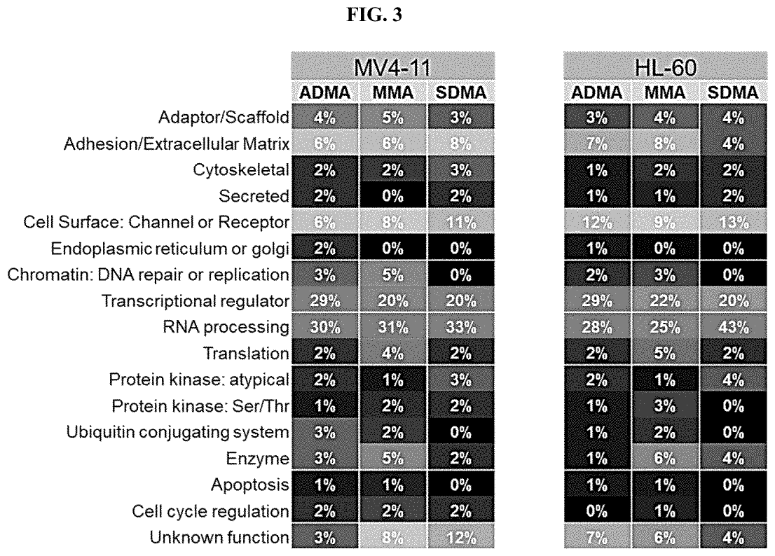

RNA binding proteins and splicing machinery are a major class of PRMT1 substrates and have been implicated in cancer biology through their biological function as well as recurrent mutations in leukemias (Bressan, G. C. et al. Arginine methylation analysis of the splicing-associated SR protein SFRS9/SRP30C. Cell Mol Biol Lett 14, 657-669, doi:10.2478/s11658-009-0024-2 (2009); Sveen, A., Kilpinen, S., Ruusulehto, A., Lothe, R. A. & Skotheim, R. I. Aberrant RNA splicing in cancer; expression changes and driver mutations of splicing factor genes. Oncogene 35, 2413-2427, doi:10.1038/onc.2015.318 (2016); Hsu, T. Y. et al. The spliceosome is a therapeutic vulnerability in MYC-driven cancer. Nature 525, 384-388, doi:10.1038/nature14985 (2015)). In a recent study, PRMT1 was shown to methylate the RNA binding protein, RBM15, in acute megakaryocytic leukemia (Zhang, L. et al. Cross-talk between PRMT1-mediated methylation and ubiquitylation on RBM15 controls RNA splicing. Elite 4, doi:10.7554/eLife.07938 (2015)). PRMT1 mediated methylation of RBM15 regulates its expression; consequently, overexpression of PRMT1 in AML cell lines was shown to block differentiation by downregulation of RBM15, thereby preventing its ability to bind pre-mRNA intronic regions of genes important for differentiation. To identify putative PRMT1 substrates, a proteomic approach (Methylscan, Cell Signaling Technology) was utilized to identify proteins with changes in arginine methylation states in response to a tool PRMT1 inhibitor, Compound D. Protein fragments from Compound D- and DSMO-treated cell extracts were immunoprecipitated using methyl arginine specific antibodies (ADMA, MMA, SDMA), and peptides were identified by mass spectrometry. While many proteins undergo changes in arginine methylation, the majority of substrates identified were transcriptional regulators and RNA processing proteins in AML cell lines treated with the tool compound (FIG. 3).

In summary, the impact of PRMT1 on cancer relevant pathways suggests inhibition may lead to anti-tumor activity, providing a novel therapeutic mechanism for the treatment of AML, lymphoma, and solid tumor indications. As described in the emerging literature, several mechanisms support a rationale for the use of a PRMT1 inhibitor in hematological and solid tumors including: inhibition of AML-ETO driven oncogenesis in leukemia, inhibition of growth promoting signal transduction in breast cancer, and modulation of splicing through methylation of RNA binding proteins and spliceosome machinery. Inhibition of Type I PRMTs including PRMT1 represents a tractable strategy to suppress aberrant cancer cell proliferation and survival.

### Biochemistry

Detailed in vitro biochemical studies were conducted with Compound A to characterize the potency and mechanism of inhibition against Type I PRMTs.

### Mechanism of Inhibition

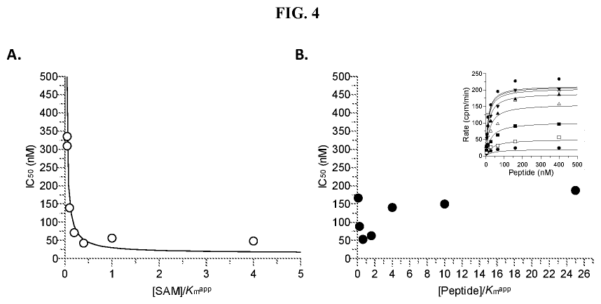

The inhibitory mechanism of Compound A for PRMT1 was explored through substrate competition experiments. Inhibitor modality was examined by plotting Compound A IC₅₀ values as a function of substrate concentration divided by its Kₘ^{app} and comparing the resulting plots to the Cheng-Prusoff relationship for competitive, non-competitive, and uncompetitive inhibition (Copeland, R. A. Evaluation of enzyme inhibitors in drug discovery. A guide for medicinal chemists and pharmacologists. Methods Biochem Anal 46, 1-265 (2005)). Compound A IC₅₀ values decreased with increasing SAM concentration indicating that inhibition of PRMT1 by Compound A was uncompetitive with respect to SAM with a Kᵢ^{app} value of 15 nM when fit to an equation for uncompetitive inhibition (FIG. 4A). No clear modality trend was observed when Compound A IC₅₀ values were plotted as a function of H4 1-21 peptide (FIG. 4B) suggesting mixed type inhibition. Further analysis was performed using a global analysis resulting in an α value of 3.7 confirming the peptide mechanism as mixed and yielding a Kᵢ^{app} value of 19 nM (FIG. 4B, inset).

### Time Dependence and Reversibility

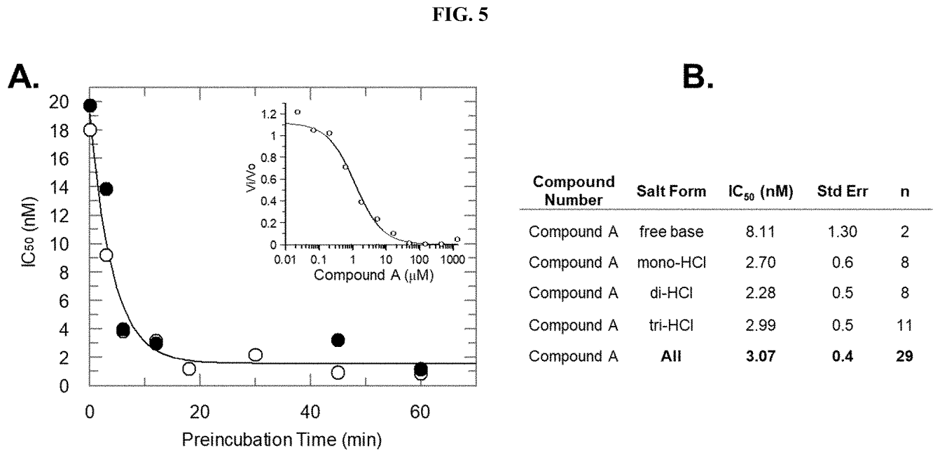

Compound A was evaluated for time dependent inhibition by measuring IC₅₀ values following varying SAM:PRMT1: Compound A preincubation time and a 20 minute reaction. An inhibitory mechanism that is uncompetitive with SAM implies that generation of the SAM:PRMT1 complex is required to support binding of Compound A, therefore SAM (held at Kₘ^{app}) was included during the preincubation. Compound A demonstrated time dependent inhibition of PRMT1 methylation evident by an increase in potency with longer preincubation time (FIG. 5A). Since time dependent inhibition was observed, further IC₅₀ determinations included a 60 minute SAM:PRMT1:Compound A preincubation and a 40 minute reaction time to provide a better representation of compound potency. These conditions yield an IC₅₀ of 3.1±0.4 nM (n=29) that is >10-fold above the theoretical tight-binding limit (0.25 nM) of the assay. Examining IC₅₀ values at varying PRMT1 concentrations revealed that the actual tight binding limit would be significantly lower than 0.25 nM potentially due to a low active fraction (FIG. 5B). The salt form of Compound A did not significantly affect the IC₅₀ value determined against PRMT1 (FIG. 5B).

Two explanations for time dependent inhibition are slow-binding reversible inhibition and irreversible inhibition. To distinguish between these two mechanisms, affinity selection mass spectrometry (ASMS) was used to examine the binding of Compound A to PRMT1. ASMS first separates bound from unbound ligand, and then detects reversibly bound ligand by MS. A 2 hr preincubation of PRMT1:SAM with Compound A was used to ensure that the time dependent complex (ESI*) was fully formed based on the profile shown in FIG. 5A) in which maximal potency was observed after 20 minutes of preincubation. Under these conditions. Compound A was detectable using ASMS. This suggests that the primary mechanism is reversible in nature, since ASMS would be unable to detect irreversibly bound Compound A. Definitive reversibility studies including off-rate analysis have not yet been performed and would further validate the mechanism.

### Crystallography

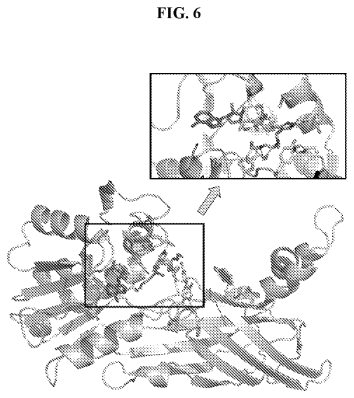

To determine inhibitor binding mode, the co-crystal structure of Compound A bound to PRMT1 and SAH was determined (2.48 Å resolution) (FIG. 6). SAH is the product formed upon removal of the methyl group from SAM by PRMT1; therefore, SAH and SAM should similarly occupy the same pocket of PRMT1. The inhibitor binds in the cleft normally occupied by the substrate peptide directly adjacent to the SAH pocket and its diamine sidechain occupies the putative arginine substrate site. The terminal methylamine forms a hydrogen bond with the Glu162 sidechain residue that is 3.6 Å from the thioether of SAH and the SAH binding pocket is bridged to Compound A by Tyr57 and Met66. Compound A binds PRMT1 through the formation of a hydrogen bond between the proton of the pyrazole nitrogen of Compound A and the acidic sidechain of Glu65; the diethoxy branched cyclohexyl moiety lies along the solvent exposed surface in a hydrophobic groove formed by Tyr57, Ile62, Tyr166 and Tyr170. The spatial separation between SAH and inhibitor binding, as well as interactions with residues such as Tyr57 could support the SAM uncompetitive mechanism revealed in the enzymatic studies. The finding that Compound A is bound in the substrate peptide pocket and that the diamine sidechain may mimic the amines of the substrate arginine residue implies that inhibitor modality may be competitive with peptide. Biochemical mode of inhibition studies support that Compound A is a mixed inhibitor with respect to peptide (FIG. 4B). The time-dependent behavior of Compound A as well as the potential for exosite binding of the substrate peptide outside of the peptide cleft could both result in a mode of inhibition that is not competitive with peptide, explaining the difference in modality suggested by the structural and biochemical studies.

### Orthologs

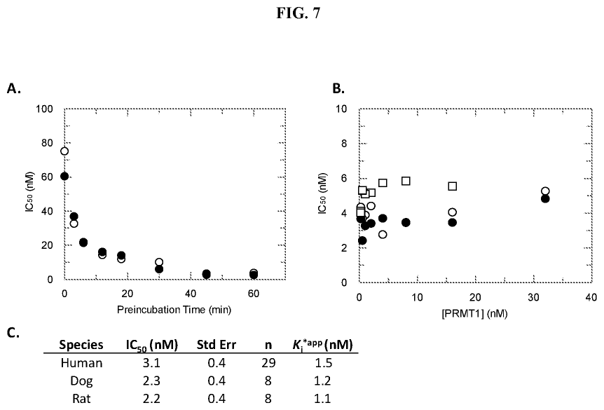

To facilitate interpretation of toxicology studies, the potency of Compound A was evaluated against the rat and dog orthologs of PRMT1. As with human PRMT1, Compound A revealed time dependent inhibition against rat and dog PRMT1 with IC₅₀ values decreasing with increasing preincubation (FIG. 7A). Additionally, no shift in Compound A potency was observed across a range of enzyme concentrations (0.25-32 nM) suggesting the IC₅₀ values measured did not approach the tight-binding limit of the assay for human, rat or dog (FIG. 7B). IC₅₀ values were determined using conditions equivalent to those used to assess human PRMT1 and revealed that Compound A potency varied <2-fold across all species (FIG. 7C).

### Selectivity

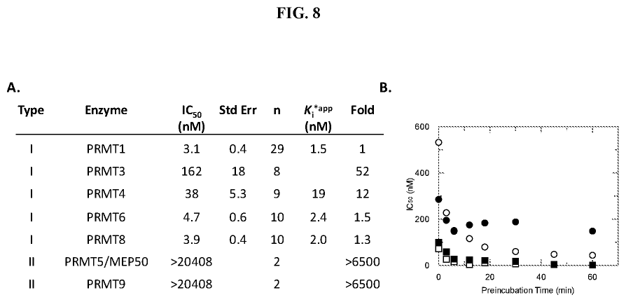

The selectivity of Compound A was assessed across a panel of PRMT family members. IC₅₀ values were determined against representative Types I (PRMT3, PRMT4, PRMT6 and PRMT8) and II (PRMT5/MEP50 and PRMT9) family members following a 60 minute SAM:Enzyme:Compound A preincubation. Compound A inhibited the activity of all Type I PRMTs tested with varying potencies, but failed to inhibit Type II family members (FIG. 8A). Additional characterization of the Type I PRMTs revealed that Compound A was a time dependent inhibitor of PRMT4, PRMT6 and PRMT8 due to the increase in potency observed following increasing Enzyme:SAM:Compound A preincubation times; whereas, PRMT3 displayed no time dependent behavior (FIG. 8B).

To further characterize selectivity of Compound A, the inhibition of twenty-one methyltransferases was evaluated at a single concentration of Compound A (10 μM, Reaction Biology). The highest degree of inhibition, 18%, was observed against PRDM9. Overall, Compound A showed minimal inhibition of the methyltransferases tested suggesting it is a selective inhibitor of Type I PRMTs (Table 2). Additional selectivity assays are described in the Safety sections.

TABLE 2
Methyltransferases tested for inhibition by Compound
A. Enzymes were assayed at a fixed concentration
of SAM (1 μM) independent of the SAM Km value.

| | | Average % |
|---|---|---|
| Methyltransferase | Substrate | Inhibition |
| PRDM9 | Histone H3 | 17.99 |
| NSD2 | Nucleosomes | 14.97 |
| MLL3 Complex | Core Histone | 13.67 |
| EZH1 Complex | Core Histone | 11.97 |
| SMYD2 | Histone H4 | 9.26 |
| PRMT3 | Histone H4 | 9.01 |
| EZH2 Complex | Core Histone | 8.17 |
| MLL2 Complex | Core Histone | 6.21 |
| SET1B Complex | Core Histone | 5.96 |
| NSD1 | Nucleosomes | 3.81 |
| G9a | Histone H3 (1-21) | 3.72 |
| SET7 | Core Histone | 3.47 |
| SETD2 | Nucleosomes | 3.15 |
| Dot1L | Nucleosomes | 2.75 |
| GLP | Histone H3 (1-21) | 1.86 |
| MLL4 Complex | Core Histone | 0.27 |
| MLL1 Complex | Nucleosomes | 0.27 |
| SUV420H1-tv2 | Nucleosomes | 0.00 |
| SUV39H1 | Histone H3 | 0.00 |
| SET8 | Nucleosomes | 0.00 |
| SUV39H2 | Histone H3 | 0.00 |

In summary, Compound A is a potent, reversible, selective inhibitor of Type I PRMT family members showing equivalent biochemical potency against PRMT1, PRMT6 and PRMT8 with IC₅₀ values ranging between 3-5 nM. The crystal structure of PRMT1 in complex with Compound A reveals that Compound A binds in the peptide pocket and both the crystal structure, as well as enzymatic studies are consistent with a SAM uncompetitive mechanism.

### Biology

### Cellular Mechanistic Effects

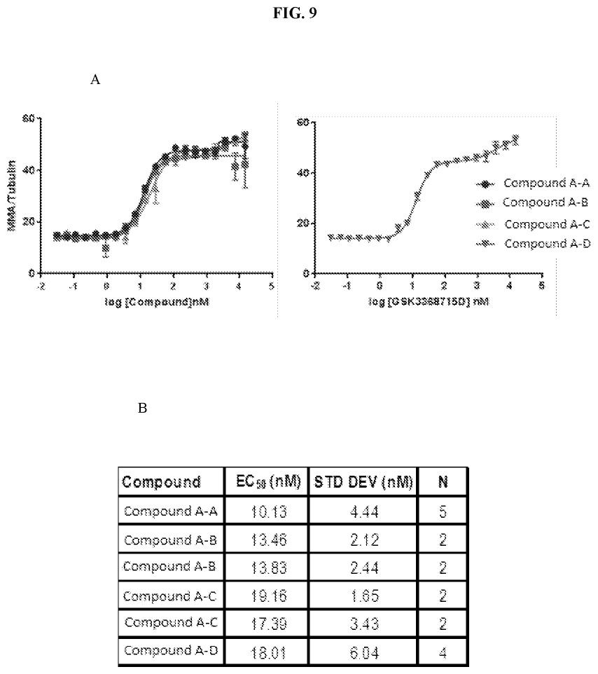

Inhibition of PRMT1 is predicted to result in a decrease of ADMA on cellular PRMT1 substrates, including arginine 3 of histone H4 (H4R3me2a), with concomitant increases in MMA and SDMA (Dhar. S. et al. Loss of the major Type I arginine methyltransferase PRMT1 causes substrate scavenging by other PRMTs. Sci Rep 3, 1311, doi:10.1038/srep01311 (2013)). To evaluate the effect of Compound A on arginine methylation the dose response associated with increased MMA was evaluated in an in-cell-western assay using an antibody to detect MMA and the cellular mechanistic EC₅₀ of 10.1±4.4 nM was determined (FIG. 9). The dose response appeared biphasic, possibly due to differential activity between the Type I PRMTs or differential potency towards a particular subset of substrates. An equation describing a biphasic curve was used to fit the data and since there was no obvious plateau associated with the second inflection over the range of concentrations tested, the first inflection was reported. Various salt forms were tested in this assay format and all demonstrated similar EC₅₀ values and are, therefore, considered interchangeable for all biology studies (FIG. 9). Additional studies were performed to examine the timing, durability, and impact on other methylation states in select tumor types as indicated below. The potency of Compound A on induction of MMA indicates that Compound A can be used to investigate the biological mechanism associated with inhibition of Type 1 PRMTs in cells.

### Type I PRMT Expression in Cancer

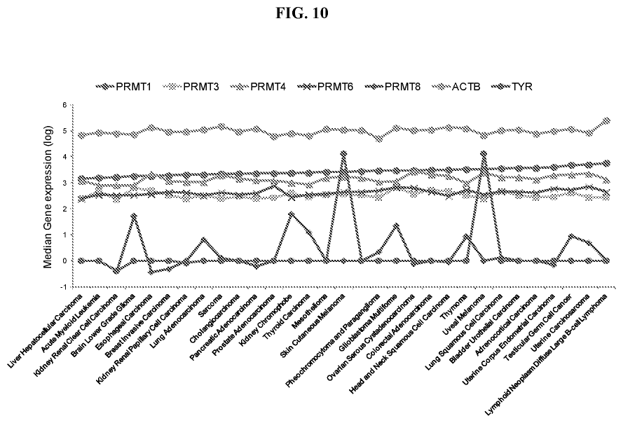

Analysis of gene expression data from multiple tumor types collected from >100 cancer studies through The Cancer Genome Atlas (TCGA) and other primary tumor databases represented in cBioPortal indicates that PRMT1 is highly expressed g in cancer, with highest levels in lymphoma (diffuse large B-cell lymphoma, DLBCL) relative to other solid and hematological malignancies (FIG. 10). Expression of ACTB, a common housekeeping gene and TYR, a gene selectively expressed in skin, were also surveyed to characterize the range associated with high ubiquitous expression or tissue restricted expression, respectively. High expression in lymphoma among other cancers provides additional confidence that the target of Compound A inhibition is present in primary tumors that correspond to cell lines evaluated in preclinical studies. PRMTs 3, 4, and 6 are also expressed across a range of tumor types while PRMT8 expression appears more restricted as predicted given its tissue specific expression (Lee, J., Sayegh, J., Daniel, J., Clarke, S. & Bedford, M. T. PRMT8, a new membrane-bound tissue-specific member of the protein arginine methyltransferase family. J Biol Chem 280, 32890-32896, doi:10.1074/jbc.M506944200 (2005)).

### Cellular Phenotypic Effects

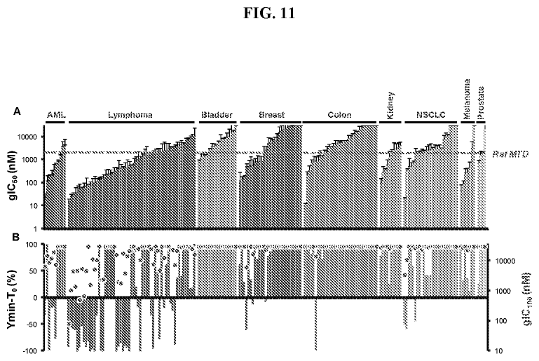

Compound A was analyzed for its ability to inhibit cultured tumor cell line growth in a 6-day growth-death assay using Cell Titer Glo (Promega) that quantifies ATP as a surrogate of cell number. The growth of all cell lines was evaluated over time across a wide range of seeding densities to identify conditions that permitted proliferation throughout the entire 6-day assay. Cells were plated at the optimal seeding density and after overnight incubation, a 20-point 2-fold titration of compound was added and plates were incubated for 6 days. A replicate plate of cells was harvested at the time of compound addition to quantify the starting number of cells (T₀). Values obtained after the 6 day treatment were expressed as a function of the T₀ value and plotted against compound concentration. The T₀ value was normalized to 100% and represents the number of cells at the time of compound addition. The data were fit with a 4 parameter equation to generate a concentration response curve and the growth IC₅₀ (gIC₅₀) was determined. The gIC₅₀ is the midpoint of the ‘growth window’, the difference between the number of cells at the time of compound addition (To) and the number of cells after 6 days (DMSO control). The growth-death assay can be used to quantify the net population change, clearly defining cell death (cytotoxicity) as fewer cells compared to the number at the time of compound addition (T₀). A negative Yₘᵢₙ−T₀ value is indicative of cell death while a gIC₁₀₀ value represents the concentration of compound required for 100% inhibition of growth. The growth inhibitory effect of Compound A was evaluated using this assay in 196 human cancer cell lines representing solid and hematological malignancies (FIG. 11).

Compound A induced near or complete growth inhibition in most cell lines, with a subset showing cytotoxic responses, as indicated by a negative Yₘᵢₙ−T₀ value (FIG. 11B). This effect was most pronounced in AML and lymphoma cancer cell lines, where 50 and 54% of cell lines showed cytotoxic responses, respectively. The total AUC or exposure (Cₐᵥₑ) calculated from the rat 14-day MTD (150 mg/kg, Cₐᵥₑ=2.1 μM) was used as an estimate of a clinically relevant concentration of Compound A for evaluation of sensitivity. While lymphoma cell lines showed cytotoxicity with gIC₁₀₀ values below 2.1 μM, many cell lines across all tumor types evaluated showed gIC₅₀ values≤2.1 μM suggesting that concentrations associated with anti-tumor activity may be achievable in patients. The dog 21-day MTD was slightly higher (25 mg/kg; total AUC or Cₐᵥₑ=3.2 μM), therefore the lower concentration from the rat provides a more conservative target for appreciating cell line sensitivity. Lymphoma cell lines were highly sensitive to Type I PRMT inhibition, with a median gIC₅₀ of 0.57 μM and cytotoxicity observed in 54%. Among solid tumor types, potent anti-proliferative activity of Compound A was observed in melanoma and kidney cancer cell lines (primarily representing clear cell renal carcinoma), however, the responses were predominantly cytostatic in this assay format (FIG. 11, Table 3).

TABLE 3
Compound A 6-day proliferation summary. gIC₅₀ ≤ 2.1 μM was used as target
based on concentration achieved in the rat 14-day MTD (150 mg/kg, Cₐᵥₑ = 2.1 μM).

| | Total | AML | Lymphoma | Bladder | Breast | Colon | Kidney | NSCLC | Melanoma | Prostate |
|---|---|---|---|---|---|---|---|---|---|---|
| Median gIC₅₀ | 2.1 [symbol] | 0.5 [symbol] | 0.57 | 5.32 | 5.95 | 5.5 | 1.66 | 2.81 | 0.28 | 1.86 |
| [symbol] | | | | | | | | | | |
| Median gIC₁₀₀ | 29. [symbol] | 16. [symbol] | 21.62 | 29.33 | 29.3 [symbol] | 29. [symbol] | 29.3 [symbol] | 29.3 [symbol] | 29.33 | 29.34 |
| [symbol] | | | | | | | | | | |
| % Cytotoxic | 23 [symbol] | 50 [symbol] | 54% | 0% | 10% | 3% | 0% | 16% | 0% | 0% |
| % gIC₅₀ < 2 | 49 [symbol] | 80 [symbol] | 69% | 28% | 41% | 29 [symbol] | 60% | 28% | 71% | 75% |
| [symbol] | | | | | | | | | | |
| % gIC₁₀₀ < 2 | 4% | 0% | 14% | 0% | 0% | 0% | 0% | 0% | 0% | 0% |
| [symbol] | | | | | | | | | | |
| Total Cell | 196 | 10 | 59 | 18 | 29 | 34 | 10 | 25 | 7 | 4 |
| [symbol] | | | | | | | | | | |

[symbol]

indicates data missing or illegible when filed

Evaluation of the anti-proliferative effects of Compound A indicates that inhibition of PRMT1 results in potent anti-tumor activity across cell lines representing a range of solid and hematological malignancies. Together, these data suggest that clinical development in solid and hematological malignancies is warranted. Prioritized indications include:
Lymphoma: cytotoxicity in 54% of cell lines
AML: cytotoxicity in 50% of cell lines
Renal cell carcinoma: gIC₅₀≤2.1 μM in 60% of cell lines
Melanoma gIC₅₀≤2.1 μM in 71% of cell lines
Breast cancer including TNBC: gIC₅₀≤2.1 μM in 41% of cell lines

### Lymphoma Biology

### Cell Mechanistic Effects

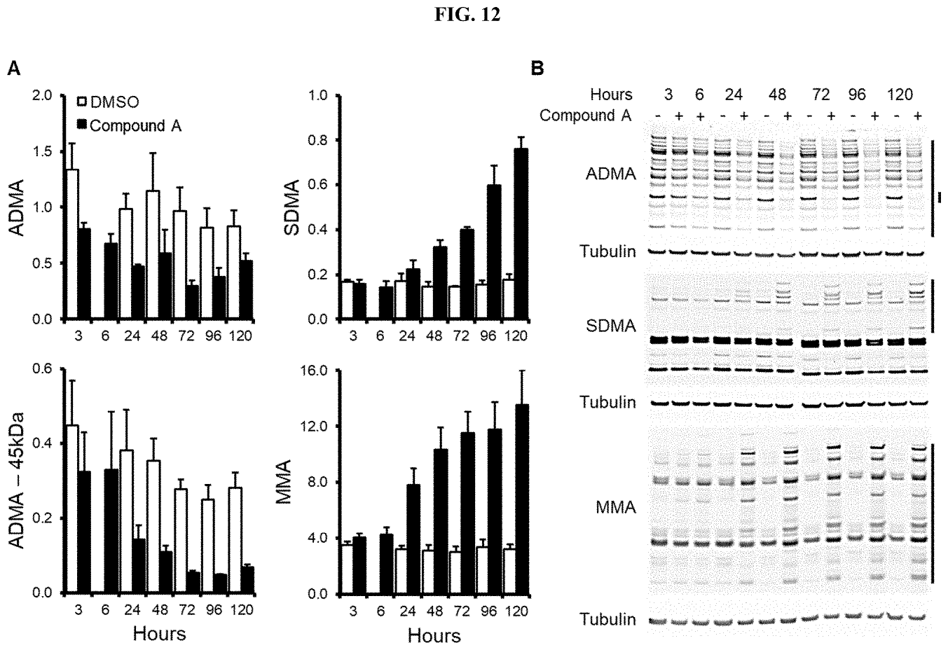

To evaluate the effect of Compound A on arginine methylation in lymphoma, a human DLBCL cell line (Toledo) was treated with 0.4 μM Compound A or vehicle for up to 120 hours after which protein lysates were evaluated by western analysis using antibodies for various arginine methylation states. As predicted, ADMA methylation decreased while MMA increased upon compound exposure (FIG. 12). An increase in levels of SDMA was also observed, suggesting that the increase in MMA may have resulted in accumulation in the pool of potential substrates for PRMT5, the major catalyst of SDMA formation. Given the detection of numerous substrates with varying kinetics, and variability of ADMA levels among DMSO-treated samples, both the full lane and a prominent 45 kDa band were characterized to assess ADMA. Increases in MMA were apparent by 24 hours and near maximal by 48 hours while decreases in the 45 kDa ADMA band required 72-96 hours to achieve maximal effect. Increases in SDMA were apparent after 48 hours of compound exposure and continued to increase through 120 hours, consistent with the potential switch from conversion of MMA to ADMA by Type I PRMTs to SDMA by Type II PRMTs (FIG. 12).

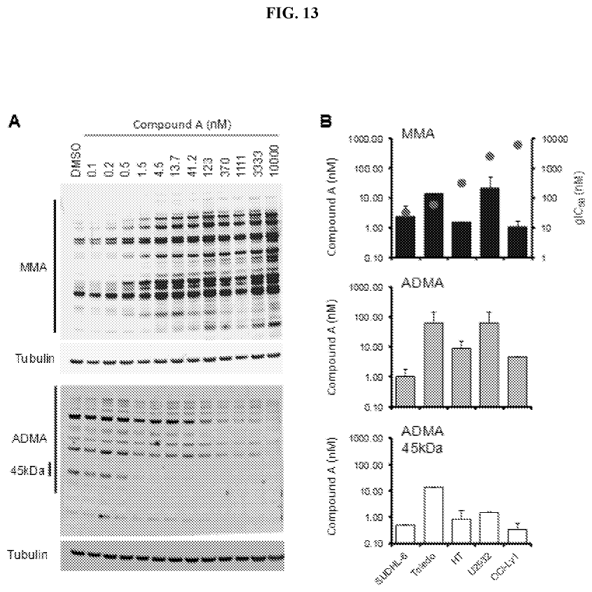

The dose response associated with Compound A effects on arginine methylation (MMA, ADMA, SDMA) was determined in a panel of lymphoma cell lines (FIG. 13). ADMA decreases were measured across the full lane and the single 45 kDa band that decreased to undetectable levels across all cell lines evaluated. Overall, concentrations required to achieve 50% of the maximal effect were similar across cell lines and did not correspond to the gIC₅₀ in the 6-day growth death assay, suggesting that the lack of sensitivity is not explained by poor target engagement.

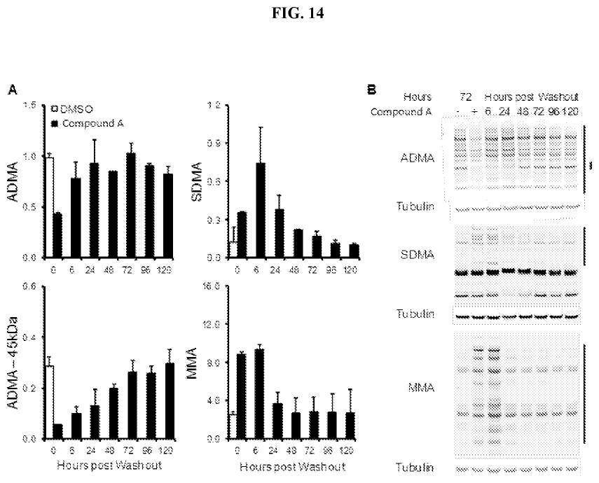

To determine the durability of global changes in arginine methylation in response to Compound A, ADMA, SDMA, and MMA levels were assessed in cells treated with Compound A after compound washout (FIG. 14). Toledo cells were cultured with 0.4 μM Compound A for 72 hours to establish robust effects on arginine methylation marks. Cells were then washed, cultured in Compound A-free media, samples were collected daily through 120 hours, and arginine methylation levels were examined by western analysis. MMA levels rapidly decreased, returning to baseline by 24 hours after Compound A washout, while ADMA and SDMA returned to baseline by 24 and 96 hours, respectively. Notably, recovery of the 45 kDa ADMA band appeared delayed relative to most other species in the ADMA western blots, suggesting the durability of arginine methylation changes by Compound A may vary by substrate. SDMA appeared to continue to increase even after 6 hours of washout. This is consistent with the continued increase observed through 120 hours without any obvious plateau (FIG. 12) coupled with the durable increase in MMA that has not yet returned to baseline after washout. Durability of each modification generally reflected the kinetics of arginine methylation changes brought about by Compound A, with MMA being the most rapid.

### Cell Phenotypic Effects

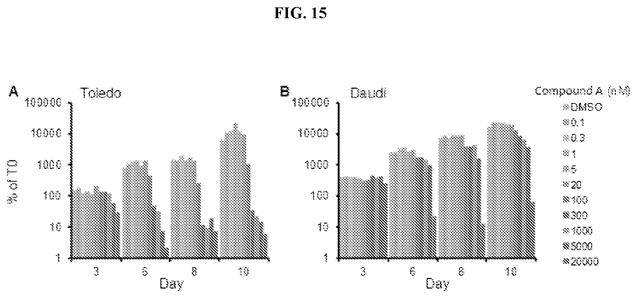

To assess the time course associated with inhibition of growth by Compound A, an extended duration growth-death assay was performed in a subset of lymphoma cell lines. Similar to the 6-day proliferation assay described previously, the seeding density was optimized to ensure growth throughout the duration of the assay, and cell number was assessed by CTG at selected timepoints beginning from days 3-10. Growth inhibition was observed as early as 6 days and was maximal by 8 days in Toledo and Daudi lymphoma cell lines (FIG. 15).

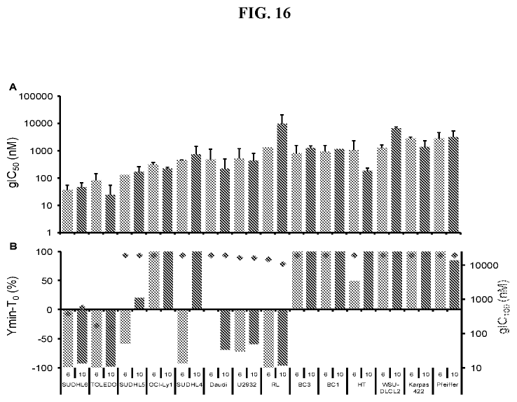

A larger set of cell lines was evaluated on days 6 and 10 to measure the effects of prolonged exposure to Compound A and determine whether cell lines that displayed a cytostatic response in the 6-day assay might undergo cytotoxicity at later timepoints. The extended time of exposure to Compound A had minimal effects on potency (gIC₅₀) or cytotoxicity (Yₘᵢₙ−T₀) across lymphoma cell lines evaluated (FIG. 16) indicating that 6-day proliferation evaluation could be utilized for assessment of sensitivity.

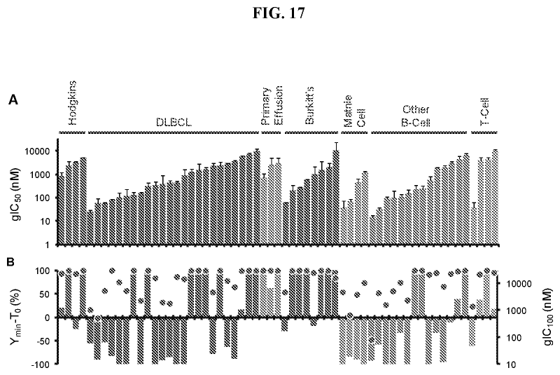

Given that growth inhibition was apparent at day 6 and prolonged exposure had minimal impact on potency or percent inhibition, a broad panel of lymphoma cell lines representing Hodgkin's and non-Hodgkin's subtypes was evaluated in the 6-day growth-death assay format (FIG. 17). All subtypes appeared equally sensitive in this format and many cell lines underwent cytotoxicity (as indicated by negative Yₘᵢₙ-T₀) independent of classification, suggesting that Compound A has anti-tumor effects in all subtypes of lymphoma evaluated.

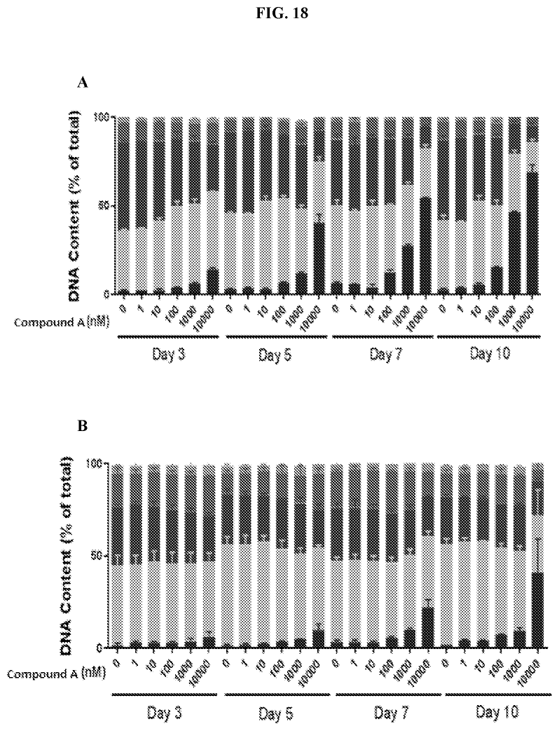

The proliferation assay results suggest that the inhibition of PRMT1 induces apparent cytotoxicity in a subset of lymphoma cell lines. To further elucidate this effect, the cell cycle distribution in lymphoma cell lines treated with Compound A was evaluated using propidium iodide staining followed by flow cytometry. Cell lines that showed a range of Yₘᵢₙ-T₀ and gIC₅₀ values in the 6-day proliferation assay were seeded at low density to allow logarithmic growth over the duration of the assay, and treated with varying concentrations of Compound A. Consistent with the growth-death assay results, an accumulation of cells in sub-G1 (<G1), indicative of cell death, was observed in Toledo cells in a time and dose dependent manner beginning after 3 days of treatment with Compound A concentrations≥1000 nM (FIG. 18). By day 7, an increase in the sub-G1 population was apparent at concentrations≥100 nM. In U2932 and OCI-Ly1, cell lines that underwent apparent cytostatic growth inhibition in the 6-day proliferation assay, this effect was only evident at 10 μM Compound A. No profound effect in any other cell cycle phase was revealed in this assay format.

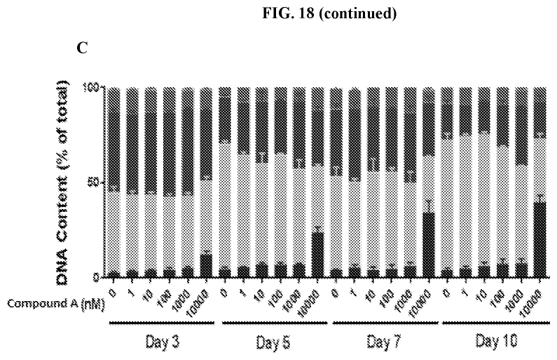

To confirm the FACS analysis of cell cycle, evaluation of caspase cleavage was performed as an additional measure of apoptosis during a 10-day timecourse. Seeding density was optimized to ensure consistent growth throughout the duration of the assay, and caspase activation was assessed using a luminescent Caspase-Glo 3/7 assay (Promega). Caspase-Glo 3/7 signal was normalized to cell number (assessed by CTG) and shown as fold-induction relative to control (DMSO treated) cells. Caspase 3/7 activity was monitored over a 10-day timecourse in DLBCL cell lines showing cytotoxic (Toledo) and cytostatic (Daudi) responses to Compound A (FIG. 19). Consistent with the profile observed in the growth-death assay, the Toledo cell line showed robust caspase activation concurrent with decreases in cell number at all timepoints, while induction of caspase activity in the Daudi cell line was less pronounced and limited to the highest concentrations of Compound A.

Together with the cell cycle profiles, these data indicate that Compound A induces caspase-mediated apoptosis in the Toledo DLBCL cell line, suggesting the cytotoxicity observed in other lymphoma cell lines may reflect activation of apoptotic pathways by Compound A.

### Anti-tumor Effects in Mouse Xenografts

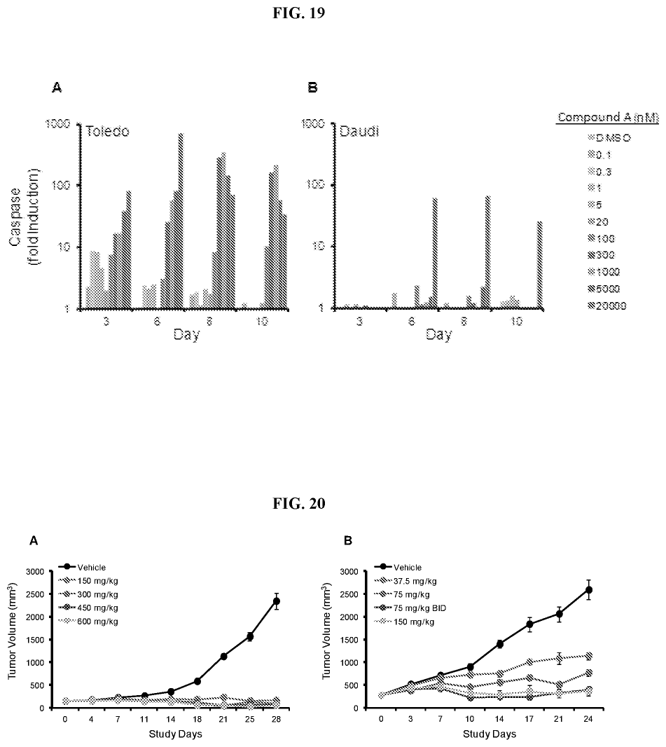

The effect of Compound A on tumor growth was assessed in a Toledo (human DLBCL) xenograft model. Female SCID mice bearing subcutaneous Toledo tumors were weighed, tumors were measured with callipers, and mice were block randomized according to tumor size into treatment groups of 10 mice each. Mice were dosed orally with either vehicle or Compound A (150 mg/kg-600 mg/kg) for 28 days daily. Throughout the study, mice were weighed and tumor measurements were taken twice weekly. Significant tumor growth inhibition (TGI) was observed at all doses and regressions were observed at doses≥300 mg/kg (FIG. 20, Table 5). There was no significant body weight loss in any dose group.

Given that complete TGI was observed at all doses evaluated, a second study was performed to test the anti-tumor effect of Compound A at lower doses as well as to compare twice daily (BID) dosing relative to daily (QD). In this second study, mice were dosed orally with either vehicle or Compound A (37.5 mg/kg-150 mg/kg) for 24 days QD or 75 mg/kg BID. In this study, BID administration of 75 mg/kg resulted in the same TGI as 150 mg/kg (95% and 96%, respectively) while ≤75 mg/kg QD resulted in partial TGI (79%) (FIG. 20, Table 5). No significant body weight loss was observed in any dose group. These data suggest that either BID or QD dosing with the same total daily dose should result in similar efficacy.

### Additional Tumor Types

### AML

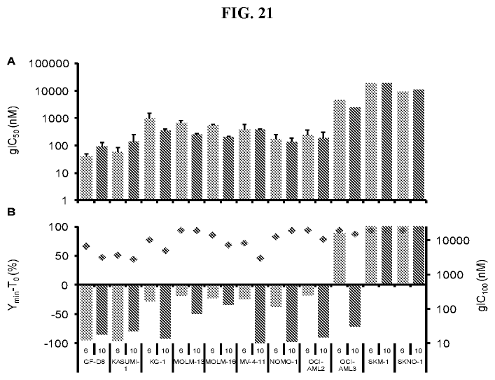

In addition to lymphoma cell lines, Compound A had potent, cytotoxic activity in a subset of AML cell lines examined in the 6-day proliferation assay (Table 3). Eight of 10 cell lines had gIC₅₀ values<2 μM, and Compound A induced cytotoxicity in 5 cell lines. Although PRMT1 interacts with the AML-ETO fusion characteristic of the M2 AML subtype (Shia, W. J. et al. PRMT1 interacts with AML1-ETO to promote its transcriptional activation and progenitor cell proliferative potential. Blood 119, 4953-4962, doi:10.1182/blood-2011-04-347476 (2012)), cell lines carrying this fusion protein (Kasumi-1 and SKNO-1) were not the only lines showing sensitivity to Compound A as measured by gIC₅₀ or that underwent cytotoxicity (Table 4, FIG. 21), therefore, the presence of this oncogenic fusion protein does not exclusively predict sensitivity of AML cell lines to Compound A.

TABLE 4
Summary of Compound A activity in AML cell lines

| | gIC₅₀ | gIC₁₀₀ | Ymin − | |
|---|---|---|---|---|
| Ceil Line | (μM) | (μM) | T₀ | Subtype |
| HL-60 | 0.02 ± 0.01 | 6.38 ± 12.83 | −33.4 | M3 |
| MV-4-11 | 0.12 ± 0.08 | 14.55 ± 4.27 | 565.6 | M5 |
| MOLM-13 | 0.21 ± 0.01 | 8.64 ± 0.39 | −100.0 | M5 |
| SKM-1 | 0.22 ± 0.11 | 11.61 ± 5.52 | −19.1 | M5 |
| KASUMI- | 0.36 ± 0.25 | 18.88 ± 10.55 | −17.7 | M2 |
| MOLM-16 | 0.65 ± 0.01 | 9.69 ± 10.58 | −68.6 | M0 |
| OCI- | 0.87 ± 0.14 | 29.33 ± 0.00 | 523.2 | M4 |
| TF-1 | 1.67 ± 0.36 | 29.33 ± 0.00 | 788.1 | M6 |
| NOMO-1 | 3.85 ± 2.10 | 29.33 ± 0.00 | 259.1 | M5 |
| SHI-1 | 4.29 ± 3.52 | 29.33 ± 0.02 | 292.0 | M5 |

Similar to studies in lymphoma, a set of cell lines was evaluated on days 6 and 10 to measure the effects of prolonged exposure to Compound A and determine whether AML cell lines that displayed a cytostatic response in the 6-day assay might undergo cytotoxicity at later timepoints. Consistent with the lymphoma result, extending time of exposure to Compound A had minimal effects on potency (gIC₅₀) or cytotoxicity (Yₘᵢₙ−T₀) across AML cell lines evaluated (FIG. 21).

### Renal Cell Carcinoma

Renal cell carcinoma cell lines had among the lowest median gIC₅₀ compared with other solid tumor types. Although none of the lines tested showed a cytotoxic response upon treatment with Compound A, all showed complete growth inhibition and 6 of 10 had gIC₅₀ values≤2 μM (Table 5). 7 of the 10 lines profiled represent clear cell renal carcinoma (ccRCC), the major clinical subtype of renal cancer.

TABLE 5
Summary of Compound A anti-proliferative
effects in renal cell carcinoma cells

| | gIC₅₀ | Ymin − | |
|---|---|---|---|
| Cell Line | (μM) | T₀ | Subtype |
| ACHN | 0.10 ± 0.05 | 96.5 | ccRCC |
| CAKI-1 | 0.28 ± 0.23 | 178.7 | ccRCC |
| G-401 | 0.35 ± 0.04 | 353.7 | Wilm's |
| 786-O | 0.59 ± 0.41 | 643.7 | ccRCC |
| SK-NEP-1 | 1.43 ± 0.86 | 25.3 | Wilm's |
| 769-P | 1.89 ± 0.82 | 119.0 | ccRCC |
| A498 | 2.73 ± 2.81 | 313.4 | ccRCC |
| G-402 | 2.89 ± 2.05 | 92.6 | Leiomyoblastoma |
| SW156 | 3.51 ± 2.01 | 346.7 | ccRCC |
| CAKI-2 | 4.23 ± 1.51 | 169.6 | ccRCC |

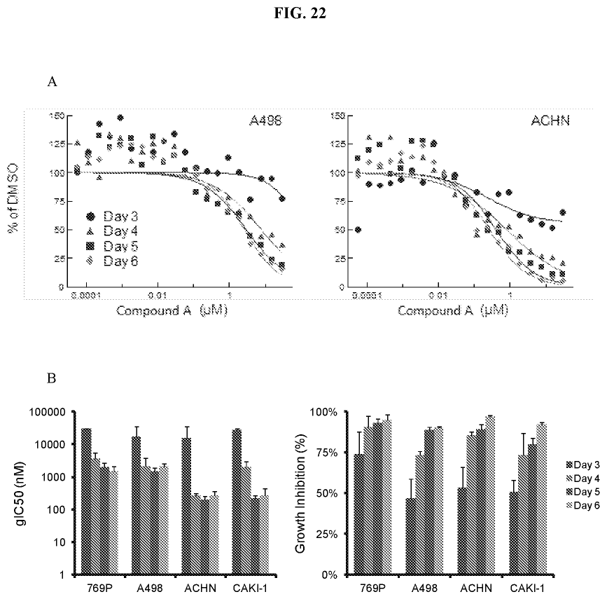

To assess the time course of growth inhibition in renal carcinoma cell lines by Compound A, cell growth was assessed by CTG in a panel of 4 ccRCC cell lines at days 3,4,5, and 6 (FIG. 22). The largest shift in activity occurred between days 3 and 4, where all cell lines showed decreases gIC₅₀ values and increases growth inhibition. Potency of Compound A (assessed by gIC₅₀) was maximal by 4 days in 3 of 4 lines and did further not change through the 6 day assay duration. Additionally, percent growth inhibition reached 100% in all cell lines evaluated. Therefore, maximal growth inhibition in ccRCC cell lines was apparent within the 6-day growth window utilized in the cell line screening strategy.

Caspase activation was evaluated during the proliferation timecourse and, consistent with the lack of overt cytotoxicity as indicated by the Yₘᵢₙ−T₀ values, caspase cleavage only occurred at the highest concentration (30 μM) indicating that apopotosis may have a minimal contribution to the overall growth inhibitory effect induced by Compound A in ccRCC cell lines.

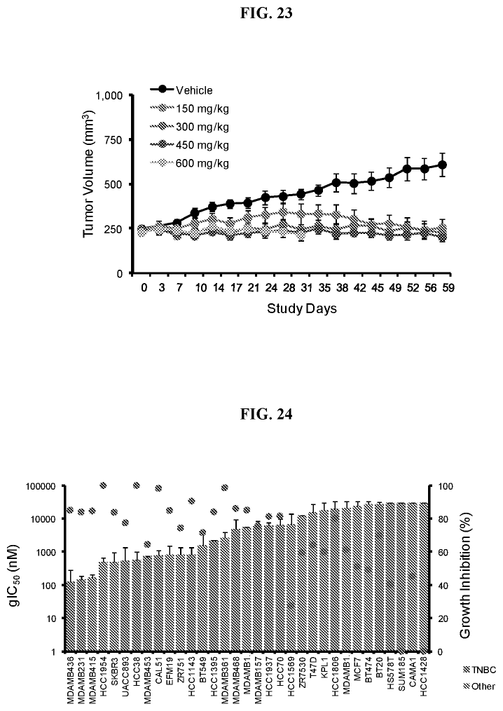

The effect of Compound A on tumor growth was assessed in mice bearing human renal cell carcinoma xenografts (ACHN). Female SCID mice bearing subcutaneous ACHN cell line tumors were weighed and tumors were measured by callipers and block randomized according to tumor size into treatment groups of 10 mice each. Mice were dosed orally with either vehicle or Compound A (150 mg/kg-600 mg/kg) for up to 59 days daily. Throughout the study, mice were weighed and tumor measurements were taken twice weekly. Significant tumor growth inhibition was observed at all doses and regressions were observed at doses≥300 mg/kg. Significant body weight loss was observed in animals treated with 600 mg/kg daily and, therefore, that dosing group was terminated on day 31 (FIG. 23, Table 6).

TABLE 6
Efficacy of Compound A in vivo

| Cell Line | | | | Body weight |
|---|---|---|---|---|
| (Tumor | Dose | TGI | | Difference |
| Type) | (mg/kg) | (Regression) | Day | (vs. vehicle) |
| Toledo | 150 QD | 99%* | 28 | −4% |
| (DLBCL) | 300 QD | 100%* (37%) | | −3% |
| | 450 QD | 100%* (58%) | | −8% |
| | 600 QD | 100%* (62%) | | −7% |
| Toledo | 37.5 QD | 63%* | 25 | −5% |
| (DLBCL) | 75 QD | 79%* | | −5% |
| | 75 BID | 95%* | | −4% |
| | 150 QD | 96%* | | −7% |
| ACHN | 150 QD | 98%* | 59 | −3% |
| (ccRCC) | 300 QD | 100%* (2%) | | −4% |
| | 450 QD | 100%* (15%) | | −7% |
| | 600 QD** | 100%* (6%) | | −17% |

*p < 0.05, two-tailed t-test
**600 QD arm of ACHN efficacy study was terminated at day 31

Together, these data suggest that 100% TGI can be achieved at similar doses in subcutaneous xenografts of human solid and hematologic tumors.

### Breast Cancer

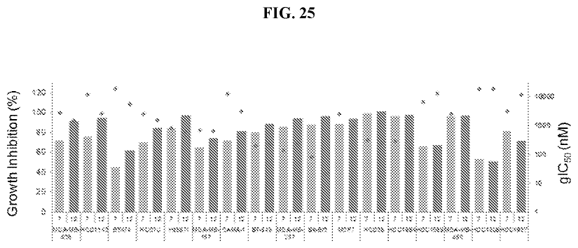

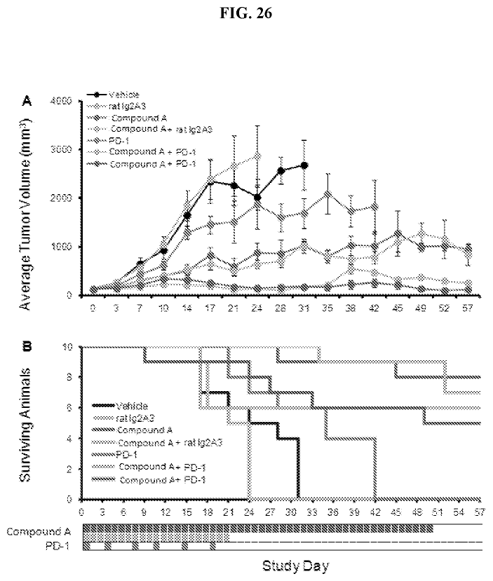

Breast cancer cell lines displayed a range of sensitivities to Compound A and in many cases, showed partial growth inhibition in the 6-day proliferation assay (FIG. 24). Cell lines representing triple negative breast cancer (TNBC) had slightly lower median gIC₅₀ values compared with non-TNBC cell lines (3.6 μM and 6.8 μM for TNBC and non-TNBC, respectively). Since the effect on proliferation by Compound A was cytostatic and did not result in complete growth inhibition in the majority of breast cancer cell lines, an extended duration growth-death assay was performed to determine whether the sensitivity to Compound A would increase with prolonged exposure. In 7/17 cell lines tested there was an increase in percent maximal inhibition by ≥10% and a ≥2-fold decrease in gIC₅₀ (FIG. 25). In the prolonged exposure assay, 11/17 cell lines had gIC₅₀≤2 μM (65%) while 7/17 (41%) met this criteria in the 7 day assay format.

### Melanoma

Among solid tumor types, Compound A had the most potent anti-proliferative effect in melanoma cell lines (FIG. 11). Six of 7 lines assessed had gIC₅₀ values less than 2 μM (Table 7). The effect of Compound A was cytostatic in all melanoma lines, regardless of gIC₅₀ value.

TABLE 7
Summary of Compound A Activity in Melanoma Cell Lines

| | gIC₅₀ | gIC₁₀₀ | Yₘᵢₙ − |
|---|---|---|---|
| Cell Line | (μM) | (μM) | T₀ |
| A375 | 0.05 ± 0.03 | 29.33 ± 0.00 | 91.9 |
| SK-MEL-5 | 0.09 ± 0.03 | 27.09 ± 3.92 | 31.8 |
| IGR-1 | 0.27 ± 0.14 | 29.33 ± 0.00 | 507.0 |
| SK-MEL-2 | 0.28 ± 0.14 | 22.37± | 35.9 |
| COLO741 | 0.43 ± 0.37 | 28.55 ± 1.40 | 12.5 |
| HT144 | 3.46 ± 2.68 | 29.33 ± 0.00 | 124.9 |
| MDA-MB- | 29.36± | 29.33 ± 0.00 | 19.1 |

### Example 2

### Combinations

Two rational approaches were undertaken to investigate potential combinations with Compound A. The second approach utilized to evaluate combinations with Compound A involved exploration of the combined benefits of immunotherapy with PRMT1 inhibition. PRMT1 has been implicated in immune regulation through modulation of the TLR receptor signaling pathway, whereby PRMT1 knock-down results in increased expression of pro-inflammatory molecules (Tikhanovich, I. et al. Dynamic Arginine Methylation of Tumor Necrosis Factor (TNF) Receptor-associated Factor 6 Regulates Toll-like Receptor Signaling. J Biol Chem 290, 22236-22249, doi:10.1074.jbc.M115.653543 (2015)). Preliminary RNA-seq studies with the PRMT1 inhibitor tool compound (Compound D) demonstrated altered expression of immune response gene families such as chemokines, cytokines, interferons, and interleukins in AML cell lines. Given the emerging clinical efficacy associated with immunotherapy, the combined anti-tumor activity of Compound A with anti-PD-1 was examined in a syngeneic immune-competent mouse model.

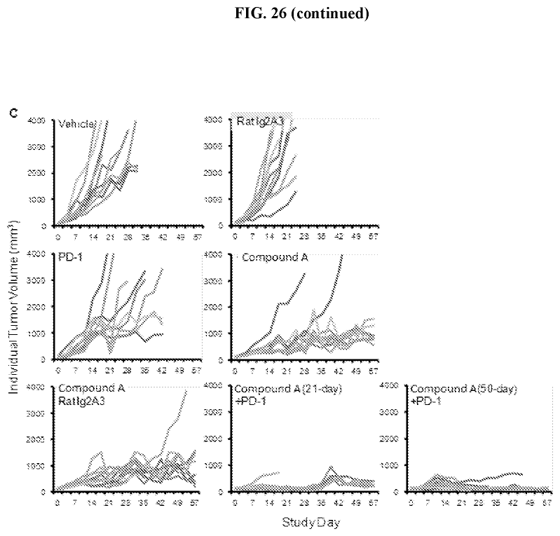

Female DBA/2N Tac mice bearing subcutaneous murine melanoma (CloudmanS91) tumors were orally administered vehicle or 300 mg/kg Compound A once daily for 3 weeks. Mice were administered anti-PD1, IgG, or corresponding vehicle 10 mg/kg intraperitoneally twice weekly for 21 days. An additional cohort was administered anti-PD1 for 21 days but continued receiving Compound A through 50 days. Tumor measurements were taken twice weekly throughout the duration of the study. Compound A alone and in combination with anti-PD1 had significant effects on tumor growth inhibition at day 21 (FIG. 26; Table 8). This effect was most profound in the Compound A/anti-PD1 combination group, where tumor regression was observed in nearly all animals (FIG. 26). Effects on bodyweight and morbidity were observed in some animals in the combination treatment groups.

TABLE 8
Statistical comparison of tumor growth inhibition at Day
21. p value (t-test) is indicated for each comparison.

| Day-21 Tumor | | | | Compound A- | Compound A- |
|---|---|---|---|---|---|
| Growth | | | Compound A- | di-HCl + | di-HCl + |
| Inhibition | rat Ig2A | PD-1 | di-HCl | rat Ig2A | PD-1 |
| Vehicle | 6E−01 | 2E−01 | 6E−03 | 5E−03 | 2E−03 |
| rat Ig2A | | 2E−01 | 2E−02 | 2E−02 | 1E−02 |
| PD-1 | | | 8E−02 | 5E−02 | 1E−02 |
| Compound | | | | 6E−01 | 4E−02 |
| A-di-HCl | | | | | |
| Compound | | | | | 1E−02 |
| A-di-HCl + | | | | | |
| rat Ig2A | | | | | |

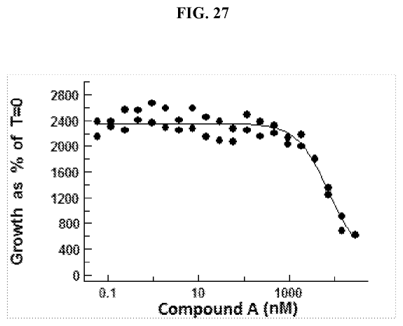

To determine whether the effects observed on tumor growth reflect the sensitivity of the cell line, the effect of Compound A on growth of CloudmanS91 cells in culture was evaluated. In a 96-well, optimized 6-day assay format, Compound A had weak effects on the growth of this mouse derived cell line (gIC₅₀=9.5 μM) suggesting that the anti-tumor activity observed in the syngeneic mouse model was not cell autonomous and may require an intact immune system (FIG. 27). Studies to confirm the contribution of the immune system to the anti-tumor effects using an immune compromised mouse xenograft model of Cloudman S91, are currently underway.

Collectively, these data suggest Compound A may engage the immune system and may synergize with immune system checkpoint modulators currently approved for use in patients as well as those under development. This mechanism could complement any direct effect on cancer cell proliferation and viability by Compound A.

### Example 3

### Combinations

Survival advantage was determined for CT-26 (colon carcinoma) tumor model mice and A20 (lymphoma) tumor model mice treated with Compound D and anti-OX40 as single agents and in combination. Mice were orally administered vehicle or 300 mg/kg Compound D once daily for 3 weeks. Mice were administered anti-OX40 (clone OX86) 5 mg/kg or corresponding vehicle intraperitoneally twice weekly for 21 days. Clone OX86 is a rat anti-mouse OX40 receptor antibody.

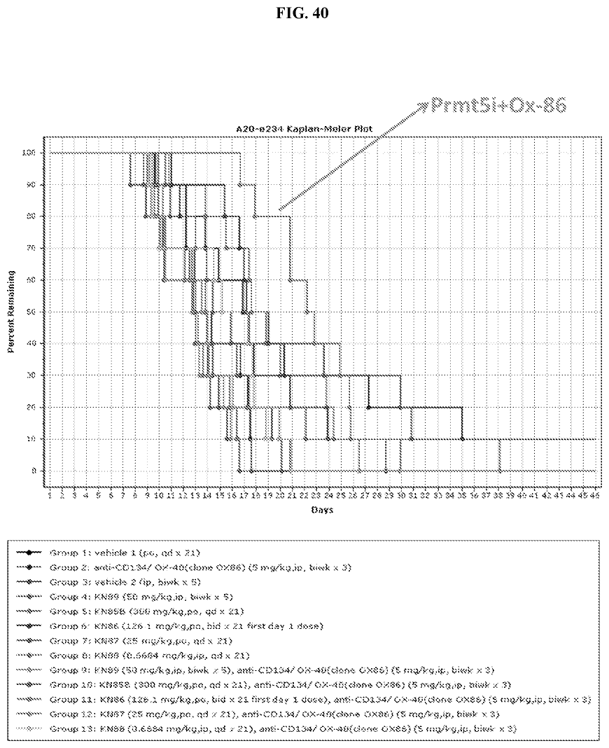

FIG. 40 shows average survival in A20 tumor model treated with corresponding vehicles (Groups 1 and 3), Compound D (Group 5), anti-OX40 (Group 2), and a combination of Compound D and anti-OX40 (Group 10).

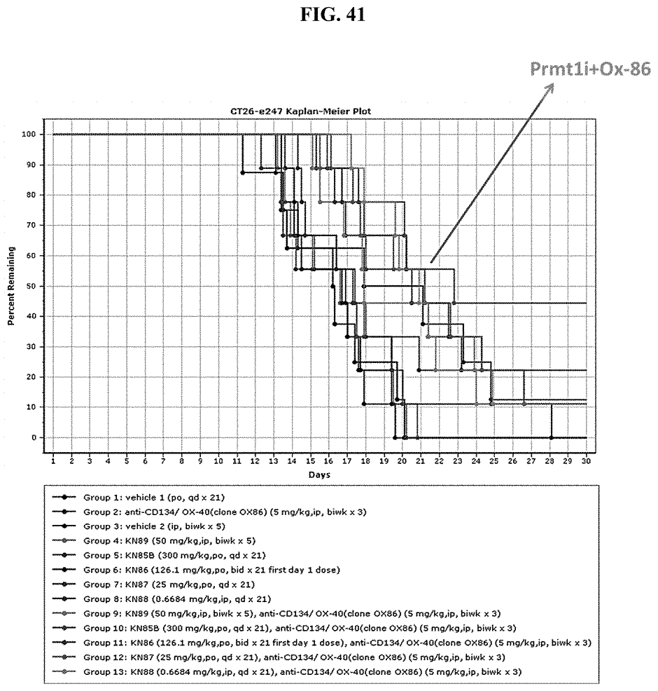

FIG. 41 shows average survival in CT-26 tumor model treated with corresponding vehicles (Groups 1 and 3), Compound A (Group 5), anti-OX40 (Group 2), and a combination of Compound D and anti-OX40 (Group 10).

Treatment of CT-26 xenograft tumors with the combination of anti-OX-40 antibody and Compound D resulted in the increase in survival, highlighting the potential synergistic interaction between two agents.

1. A combination of a Type I protein arginine methyltransferase (Type I PRMT) inhibitor and an immuno-modulatory agent selected from: an anti-PD-1 antibody or antigen binding fragment thereof, an anti-PDL1 antibody or antigen binding fragment thereof, and an anti-OX40 antibody or antigen binding fragment thereof.

2. The combination of claim 1, wherein the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor.

3. The combination of claim 1, wherein the Type I PRMT inhibitor is a compound of Formula (I): 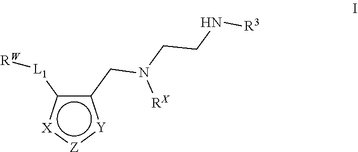
or a pharmaceutically acceptable salt thereof,
wherein
X is N, Z is NR⁴, and Y is CR⁵; or
X is NR⁴, Z is N, and Y is CR⁵; or
X is CR⁵, Z is NR⁴, and Y is N; or
X is CR⁵, Z is N, and Y is NR⁴;
R^{X} is optionally substituted C₁₋₄ alkyl or optionally substituted C₃₋₄ cycloalkyl;
L₁ is a bond, —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—, —C(═NOR^{A})—, —C(═NR^{B})N(R^{B})—, —NR^{B}C(═NR^{B})—, —C(S)—, —C(S)N(R^{B})—, —NR^{B}C(S)—, —S(O)—, —OS(O)₂—, —S(O)₂O—, —SO₂—, —N(R^{B})SO₂—, —SO₂N(R^{B})—, or an optionally substituted C₁₋₆ saturated or unsaturated hydrocarbon chain, wherein one or more methylene units of the hydrocarbon chain is optionally and independently replaced with —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—, —C(═NOR^{A})—, —C(═NR^{B})N(R^{B})—, —NR^{B}C(═NR^{B})—, —C(S)—, —C(S)N(R^{B})—, —NR^{B}C(S)—, —S(O)—, —OS(O)₂—, —S(O)₂O—, —SO₂—, —N(R^{B})SO₂—, or —SO₂N(R^{B})—;
each R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom;
each R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or an R^{B} and R^{W} on the same nitrogen atom may be taken together with the intervening nitrogen to form an optionally substituted heterocyclic ring;
R^{W} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl; provided that when L₁ is a bond, R^{W} is not hydrogen, optionally substituted aryl, or optionally substituted heteroaryl;
R³ is hydrogen, C₁₋₄ alkyl, or C₃₋₄ cycloalkyl;
R⁴ is hydrogen, optionally substituted C₁₋₆ alkyl, optionally substituted C₂₋₆ alkenyl, optionally substituted C₂₋₆ alkynyl, optionally substituted C₃₋₇ cycloalkyl, optionally substituted 4- to 7-membered heterocyclyl; or optionally substituted C₁₋₄ alkyl-Cy;
Cy is optionally substituted C₃₋₇ cycloalkyl, optionally substituted 4- to 7-membered heterocyclyl, optionally substituted aryl, or optionally substituted heteroaryl; and
R⁵ is hydrogen, halo, —CN, optionally substituted C₁₋₄ alkyl, or optionally substituted C₃₋₄ cycloalkyl.

4. (canceled)

5. (canceled)

6. The combination of claim 1, wherein the Type I PRMT inhibitor is Compound A: 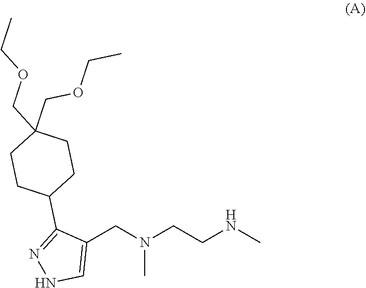
or a pharmaceutically acceptable salt thereof.

7. The combination of claim 1, wherein the immuno-modulatory agent is an antagonist anti-PD-1 antibody or antigen binding fragment thereof.

8. The combination of claim 7, wherein the anti-PD-1 antibody is pembrolizumab or nivolumab.

9. The combination of claim 1, wherein the immuno-modulatory agent is an agonist anti-OX40 antibody or antigen binding fragment thereof.

10. (canceled)

11. (canceled)

12. The combination of claim 9, wherein the immuno-modulatory agent is an anti-OX40 antibody or antigen binding fragment thereof comprising a variable heavy chain sequence having at least 90% sequence identity to the amino acid sequence set forth in SEQ ID NO:5 and a variable light chain sequence having at least 90% sequence identity to the amino acid sequence set forth in SEQ ID NO: 11.

13. A combination of a Type I protein arginine methyltransferase (Type I PRMT) inhibitor and an immuno-modulatory agent, wherein the Type I PRMT inhibitor is Compound A: 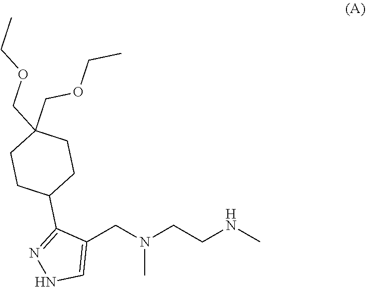
or a pharmaceutically acceptable salt thereof, and the immuno-modulatory agent is an anti-PD1 antibody or antigen binding fragment thereof, wherein the anti-PD1 antibody is selected from pembrolizumab or nivolumab.

14. (canceled)

15. A combination of a Type I protein arginine methyltransferase (Type I PRMT) inhibitor and an immuno-modulatory agent, wherein the Type I PRMT inhibitor is Compound A: 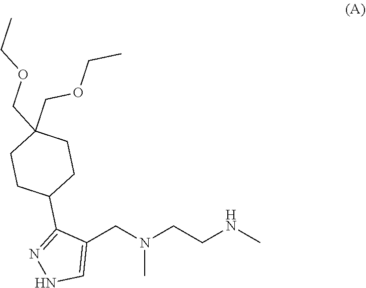
or a pharmaceutically acceptable salt thereof, and the immuno-modulatory agent is an anti-OX40 antibody or antigen binding fragment thereof comprising a variable heavy chain sequence having at least 90% sequence identity to the amino acid sequence set forth in SEQ ID NO:5 and a variable light chain sequence having at least 90% sequence identity to the amino acid sequence set forth in SEQ ID NO: 11.

16. A method of treating cancer in a human in need thereof, the method comprising administering to the human a combination of claim 1, together with at least one of: a pharmaceutically acceptable carrier and a pharmaceutically acceptable diluent, thereby treating the cancer in the human.

17.-32. (canceled)

33. The method of claim 16, wherein the Type I PRMT inhibitor and the immuno-modulatory agent are administered to the patient in a route selected from: simultaneously, sequentially, in any order, systemically, orally, intravenously, and intratumorally.

34. The method of claim 16, wherein the Type I PRMT inhibitor is administered orally.

35. The method of claim 16, wherein the cancer is melanoma, lymphoma, or colon cancer.

36.-37. (canceled)
